(19) United States
(12) Patent Application Publication
(10) Pub. No.: US 2016/0030726 A1
(43) Pub. Date: Feb. 4, 2016

(54) METHODS OF DELIVERING NANOSHELLS INTO SEBACEOUS GLANDS

(71) Applicant: THE REGENTS OF THE UNIVERSITY OF CALIFORNIA, Oakland,, CA (US)

(72) Inventors: Byeonghee Hwang, Goleta, CA (US); Samir Mitragotri, Santa Barbara, CA (US)

(21) Appl. No.: 14/777,291

(22) PCT Filed: Mar. 17, 2014
(86) PCT No.: PCT/US2014/030608

§ 371 Date: Sep. 15, 2015

Related U.S. Application Data
(60) Provisional application No. 61/793,233, filed on Mar. 15, 2013.

(51) Int. Cl. A61M 37/00; A61K 9/51; A61N 5/06; A61K 41/00

(52) U.S. Cl. CPC A61M 37/0092; A61K 41/0047; A61K 9/51; A61N 5/062; A61N 2005/067

(57) ABSTRACT

Improved methods for treating a sebaceous gland disorder, such as acne, are described. The methods include a) cleaning the skin site with a solvent by applying immersion low frequency ultrasound to the site; b) delivering nanoshell particles into the infundibula and sebaceous glands over a period of time, by applying iontophoresis, low frequency ultrasound, or electroporation, or a combination thereof, preferably administering immersion low frequency ultrasound; and c) thermally activating the nanoshell particles to modify or destroy the infundibula and sebaceous gland are provided. A sufficient amount of the nanoshell particles infiltrates spaces about the sebaceous glands and is exposed to energy to cause the particles to become thermally activated. Photothermal activation of the nanoshell particles brings about a physiological change in the sebaceous gland, thereby treating the sebaceous gland disorder. Preferably, the sebaceous gland is destroyed. There is minimal to no destruction of normal adjacent epidermal and dermal structures.

### CROSS-REFERENCE TO RELATED APPLICATION

This application claims priority to U.S. Ser. No. 61/793,233, entitled “Methods of Delivering Nanoshells into Sebaceous Glands” to Samir Mitragotri and Byeonghee Hwang, filed Mar. 15, 2013. The disclosure of which is incorporated herein by reference.

### FIELD OF THE INVENTION

The present invention generally relates to methods for treating sebaceous gland disorders, such as acne. In particular, this invention relates to the use of low frequency ultrasound in the transport of nanoshells into the follicles and follicular appendages.

### BACKGROUND OF THE INVENTION

Acne vulgaris is the most common skin disease that afflicts the majority of teenagers, along with a significant number of men and women of adult age. Acne vulgaris can occur anywhere on the body, most often on oily areas of the skin having high sebaceous gland concentration. These areas include the face, ears, behind the ears, chest, back, and occasionally the neck and upper arms. One causative factor for acne is increased activity of the sebaceous glands and the epithelial tissue lining the infundibulum, in which bacterial invasions cause inflamed and infected sacs to appear. Among the bacteria flora present are anaerobic, Gram positive organisms called Proprionibacterium acnes.

The sebaceous glands are connected to the hair follicle. The combination of the follicle and sebaceous gland is sometimes referred to as a “pilosebaceous unit.” In healthy skin, the sebaceous glands produce sebum which flows out of the skin through the follicle. In diseased skin, the follicle becomes plugged with dead skin cells, dirt, oil, sebum, bacteria, viruses, etc.

When there is a build-up in the follicle, inflammation and often rupture of the hair follicles ensues, leading to gross inflammation, pus (a “whitehead”), pain, bleeding, and/or eventual scarring. If the acne lesion consists of an accumulated unruptured plug within a hair follicle, a “blackhead” forms. If the follicle ruptures superficially, a small pustule forms that often heals after a few weeks without scarring. If the follicle ruptures within the mid or deep dermis, a painful cystic abscess forms. Cystic acne usually heals with permanent and disfiguring scars.

The most common treatments for acne are oral retinoids, such as retinoic acid (Accutane®), which inhibit sebaceous gland function. However, while the retinoids are effective in treating acne, oral retinoids are both toxic and teratogenic. Many other topical treatments including creams, gels, and various cleansing pads have been used to treat acne. The major drawback of topical treatments is that the creams or other substances do not treat the underlying cause of acne and must be continually used.

U.S. Pat. Nos. 6,635,075 and 6,245,093 to Li et al. disclose devices for treating acne including the Zeno™ device produced by Tyrell, Inc. of Houston, Tex. This device passes heat through acne diseased skin or heats the surface of the skin but does not apply heat below the surface of the skin. However, these devices are not effective, are uncomfortable to use, and cannot treat severe acne.

Laser dermatology treatments have been used to treat acne. These treatments produce a permanent anatomic, microsurgical effect on the skin. However, these treatments are generally considered to be ineffective. Further, they do not specifically target the sebaceous glands.

U.S. Pat. No. 6,183,773 to Anderson discloses the use of laser sensitive dyes in combination with laser treatment for the treatment of acne. The laser sensitive dyes are topically applied to the skin. However, dyes generally do not display selectivity toward the follicle and have substantial concentration in the non-follicular tissue, for example, stratum corneum and the epidermis.

U.S. Publication No. 2012/0059307 to Harris et al., discloses nanoparticle formulations useful for treating various skin conditions, for example, a sebaceous gland disorder. Harris et al. uses plasmonic nanoparticles to induce selective thermo-modulation in a target tissue, such as the sebaceous gland Harris et al. discloses high frequency ultrasound to force the particles into the follicles. However, Harris discloses the use of high frequency ultrasound in direct contact with the skin surface.

Therefore, there is a need for improved devices and methods for the treatment of the underlying causes of acne, particularly for treatments that directly target the sebaceous glands.

It is therefore an object of the invention to provide an improved method for treating sebaceous gland disorders, including acne.

It is another object of the invention to provide a method for selectively targeting the sebaceous glands which is able to modify or destroy the sebaceous glands.

### SUMMARY OF THE INVENTION

Improved methods for treating a sebaceous gland disorder, such as acne, are described herein. The methods include cleaning the skin site with a solvent by applying low frequency ultrasound to the site. Subsequently or simultaneously, nanoshell particles are delivered into the infundibula and sebaceous glands via microjets produced locally near the skin surface or by applying pressure, preferably via low frequency ultrasound, at the skin site. The ultrasound modalities are selected to push the nanoshell particles into the infundibula and sebaceous glands without damaging the surrounding skin, the follicle root, or any other tissue surrounding the hair follicle. In a final step, energy at a wavelength that matches the absorption spectrum of the nanoshell particle is directed at the nanoshell particle, preferably via a laser, to selectively thermally activate the nanoshell particles, and thereby modify or destroy the infundibula and sebaceous gland, without damaging the surrounding skin, the follicle root, or any other tissue surrounding the hair follicle.

In one embodiment, cleaning the afflicted skin site and delivery of the nanoshell particles are consecutive steps. In another embodiment, these steps are performed simultaneously. The ultrasound energy generates cavitation bubbles in the cleaning solvent, inside as well as outside the hair follicles. The cavitation bubbles inside the hair follicle collapse and transfer their energy into the follicle plug. In a preferred embodiment, cleaning results in dislodging the follicle plug. In one embodiment, cleaning results in loosening of the plug. In one embodiment, cleaning modifies the plug, such that pores and/or channels are formed within the plug.

The nanoshell particles may be delivered to the sebaceous gland by any suitable means, including but are not limited to injection, liposome encapsulation technology, iontophoresis, ultrasonic technology, electroporation, other means for delivery of nanoparticles into the dermal region of the skin, e.g., pharmaceutically acceptable carriers, or combination thereof. The nanoshell particles preferably contain a silica core, a gold shell layer, and an outer layer of polyethylene glycol. The wavelength of maximum optical absorption (λₘₐₓ) of the particle is determined by the ratio of the core radius to the shell thickness for a particle of given core and shell materials and particle diameter. Each of these variables (i.e., core radius and shell thickness) can be easily and independently controlled during fabrication of the nanoshells. Varying the shell thickness, core diameter, and the total nanoparticle diameter allows the optical properties of the nanoshells to be tuned over the visible and near-IR spectrum. Preferably, the nanoshells can be photothermally excited using wavelengths ranging from about 700 nm to about 1300 nm.

Following administration of the nanoparticles to the sebaceous glands, an energy (light) source, e.g., a laser or filtered broadband intense pulsed light, is matched with a wave-length to the absorption spectrum of the nanoshell particle to selectively thermally activate the nanoshell particles. When the nanoshell particles are activated, they heat up. The heat is transferred to the surrounding sebaceous glands which may be destroyed. However, there is minimal to no destruction of normal adjacent epidermal and dermal structures. The thermal degradation of the sebaceous glands modifies the pore opening to the infundibulum such that the geometry, e.g., the shape, of the opening is permanently altered. The constriction, closure, or opening of the pore prevents accumulation of dirt, oils, bacteria, or viruses in that follicle. The opening to the infundibulum may be altered such that pore blockage, resulting in a blackhead or white head, will not occur. Alternately, the opening to the infundibulum may be opened. Preferably, the sebaceous glands are destroyed, thereby preventing the reoccurrence of acne. One of skill in the art can assess and adjust parameters, such as the concentration of the nanoshells in the composition delivered to the skin site, and the energy emitted by the laser, to elicit the desired effect. This is determined on a patient by patient basis.

### BRIEF DESCRIPTION OF THE DRAWINGS

FIG. 1 is a cross sectional side view of an apparatus for intradermal delivery of nanoshell particles using ultrasound.

FIG. 2 is a cross sectional side view of a Franz Cell apparatus, which was used in the Example to measure infusion of nanoshell particles into human cadaver epidermis.

### DETAILED DESCRIPTION OF THE INVENTION

Methods for treating sebaceous gland disorders, such as acne, are described herein. The methods include delivering nanoshell particles into the sebaceous gland, and thermally activating the particles with an energy source, to treat the disorders. The methods preferably include a cleaning step that is performed prior to or during delivery of the nanoshell particles. Physical means, for example ultrasound, is used during the cleaning step and to deliver the nanoshell particles, for enhanced penetration of the solvent and particles. Preferably, treatment of the disorders includes eliminating, inhibiting, or preventing occurrence or reoccurrence of the skin disorder.

Examples of sebaceous gland disorders that can be treated using the methods described herein include sebaceous gland hyperplasia, acne vulgaris and acne rosacea. Preferably, the sebaceous gland disorder to be treated is acne.

### I. DEFINITIONS

“Sebaceous gland disorders”, as used herein, refers to those sebaceous gland disorders which can be treated by a photothermal activatable nanoshell. Examples of such sebaceous gland disorders include, but are not limited to, sebaceous gland hyperplasia, acne vulgaris and acne rosacea.

“Modify”, as used herein with respect to the sebaceous glands, refers to enlargement or constriction of the opening to the infundibula and/or the sebaceous glands. Further, modifying the sebaceous gland also refers to altering the opening to the infundibulum such that pore pluggage will not occur, e.g., the infundibulum is reshaped such that excess sebum, oils, dirt and bacteria will not cause pore pluggage to occur, resulting in a black head (open comedone) or white head (milium or closed comedone).

“Pluggage”, as used herein, refers to obstruction of the pores by the buildup of sebum, dirt, bacteria, mites, oils, and/or cosmetics in the pore, e.g., about the infundibulum and within the sebaceous gland.

“Thermal activation”, as used herein, refers to the capability of producing a desired pharmacological, cellular, electrical, or mechanical effect in a medium (i.e. a predetermined change) when heat energy is absorbed. Photothermal activation of the nanoshell particles causes the particles to be heated, thereby heating the local area, preferably selectively with a significant temperature increase of such that unwanted material, e.g., tissues, oils, bacteria, viruses, dirt, etc. in the hair follicle is degraded. Additionally, this treatment can cause the opening to the hair follicle to become modified, e.g., the pore opening is enlarged or the pore opening is constricted or closed. Consequently, alteration of the pore opening, such as enlargement of the pore opening, a change in the pore shape, or constriction of the pore opening prevents unwanted dirt, bacteria, viruses and/or oils from building up in the treated area, e.g., the infundibulum. Additionally, the process can cause cell death in the sebaceous gland, thereby decreasing production of sebum.

### II. METHODS OF TREATING SEBACEOUS GLAND DISORDERS

A method to selectively modify or destroy sebaceous glands has been developed. The method includes the following steps: (1) cleaning the skin site with a solvent by applying low frequency ultrasound to the site; (2) delivering nanoshell particles into the infundibula and sebaceous glands over a period of time, by applying iontophresis, low frequency ultrasound, or electroporation, or a combination thereof; and (3) thermally activating the nanoshell particles to modify or destroy the infundibula and sebaceous gland are provide.

In one embodiment, cleaning the afflicted skin site and delivery of the nanoshell particles are consecutive steps. In another embodiment, these steps are performed simultaneously. The method uses devices, for example, ultrasound, iontophoresis, or electroporation to provide a driving force for the solvent to clean the skin as well as transport of the nanoshells.

Application of Low Frequency Ultrasound

In a preferred embodiment, the device is a low frequency ultrasound device which induces cavitation. Ultrasound is defined as sound at a frequency of between 20 kHz and 10 MHz, with intensities of between 0.1 and 100 W/cm². Ultrasound is preferably administered at frequencies of less than or equal to about 2.5 MHz to induce cavitation of the skin to enhance transport. As used herein, “low frequency” ultrasound is ultrasound at a frequency that is less than 1 MHz, more typically in the range of 20 to 100 KHz, preferably, in the range of 20 kHz to 50 kHz, more preferably about 40 kHz, which can be applied continuously or in pulses, for example, 100 msec pulses every second, at intensities in the range of between 0.1 and 100 W/cm², preferably between 1 W/cm² and 30 W/cm². Exposures are typically for between 1 second and 10 minutes, preferably between 2 seconds and 5 minutes, more preferably between 5 seconds and 1 minute, but may be shorter and/or pulsed. It should be understood that although the normal range of ultrasound begins at 20 kHz, one could achieve comparable results by varying the frequency to slightly more or less than 20 kHz. The intensity should not be so high as to raise the skin temperature more than about one to two degrees Centigrade.

Application of low-frequency ultrasound generates cavitation bubbles in the cleaning solvent in which the horn is immersed, inside as well as outside the hair follicles. In a preferred embodiment, cleaning results in dislodging the follicle plug. In one embodiment, cleaning results in loosening of the plug. In one embodiment, cleaning modifies the plug, such that pores and/or channels are formed within the plug.

Delivery of Nanoshell Particles

Topically-applied nanoshell particles initially enter the infundibula and later distribute to the sebaceous glands. It is possible to actively drive these particles into the follicles by massage, pressure, ultrasound, or iontophoresis, after topically applying the particles to the skin surface. Preferably, ultrasound radiation is used to drive the particles into the follicles. The ultrasound radiation is effective in generating jets; the jets drive the nanoshell suspension into the hair follicles and its appendages in the skin.

The nanoshells are typically formed with a core of a dielectric or inert material, such as silica, coated with a metal. These can be photothermally excited using radiation such as near infrared light (approximately 700 to 1300 nm). The combined diameter of the shell and core of the nanoshells ranges from the tens to the hundreds of nanometers.

The method further involves selective thermal activation of the nanoshell particles, whereby an energy (light) source, e.g., a laser, is matched with a wave-length to the absorption spectrum of the nanoshell particle. Upon excitation, the nanoshells emit heat. Because the nanoshells are selectively concentrated within or about the undesired deposits, the deposits are degraded by the heat generated from the energy activated material. There is minimal to no destruction of normal adjacent epidermal and dermal structures. Preferably, photothermally activation of the nanoshell particles brings about a physiological change in the hair follicle, thereby treating the sebaceous gland disorder. Suitable energy sources include electromagnetic sources including, energy emitted by the sun, flash lamp based sources and lasers. The energy source can be a pulsed or continuous wave energy source.

A. Cleaning Step

The methods for treating sebaceous gland disorders include cleaning the afflicted skin site. Cleaning facilitates deeper penetration of the nanoshell particles in the sebaceous gland. Deep penetration of the sebaceous glands can be determined by observation under a microscope, such as described in the Examples. Deep penetration as used herein generally refers to sebaceous glands that show significant damage following thermal irradiation of nanoshell particles within the glands.

The cleaning step is carried out by applying a solvent to the site. Any suitable solvent that is safe for administration to the skin may be applied in the cleaning step. Suitable solvents include but are not limited to water, acetone, isopropyl alcohol (e.g. 60-75% (v/v) solution of isopropyl alcohol in water), ethanol, dimethylsulfoxide (DMSO), hydrogen peroxide, benzoyl peroxide, benzoyl alcohol or combinations thereof.

The cleaning step includes the application of ultrasound or another force to loosen, dislodge, destroy, or otherwise desirably modify the blockage within a follicle. Wiping, rubbing and massage are not sufficient forces for the cleaning step.

Preferably, low-frequency ultrasound is applied to clean the site. The ultrasound waves may cause expansion and compression of the hair follicle, with the formation and collapse of cavitation microbubbles in the fluid near the skin surface. The collapsing microbubbles cause formation of microjets incident toward the skin surface. These cause a deeper penetration of solvent into the follicle. Furthermore, the ultrasound waves provide energy to the skin surface which may heat the solvent and skin to a temperature sufficient to loosen, dislodge, destroy, or otherwise desirably modify the blockage within a follicle.

In one embodiment, the cleaning step is carried out prior to delivering the nanoshell suspension. The solvent is typically delivered from a reservoir in the ultrasound transducer. After the cleaning step, then the cleaning solvent is discarded from the reservoir, and subsequently a nanoshell suspension is introduced into the reservoir.

In another embodiment, the nanoshell suspension contains the cleaning solvent. The surface of the skin is cleaned during delivery of the nanoshell. The nanoshell suspension may contain a solvent, present in an amount ranging from about 10% to about 90% by weight of the suspension, preferably from about 30% to about 70% by weight of the suspension. In this embodiment, following the cleaning step, typically, the nanoshell suspension is delivered for a second time to the skin site.

a. Transducer

Any suitable ultrasound transducer that is able to deliver the solvent at a variety of sites on the patient's skin can be used in the cleaning step. Preferably the transducer is an immersion ultrasound transducer. Typically the same ultrasound transducer that is used in the cleaning step is used in the nanoshell delivery step(s). Typically the ultrasound transducer is a hand-held transducer.

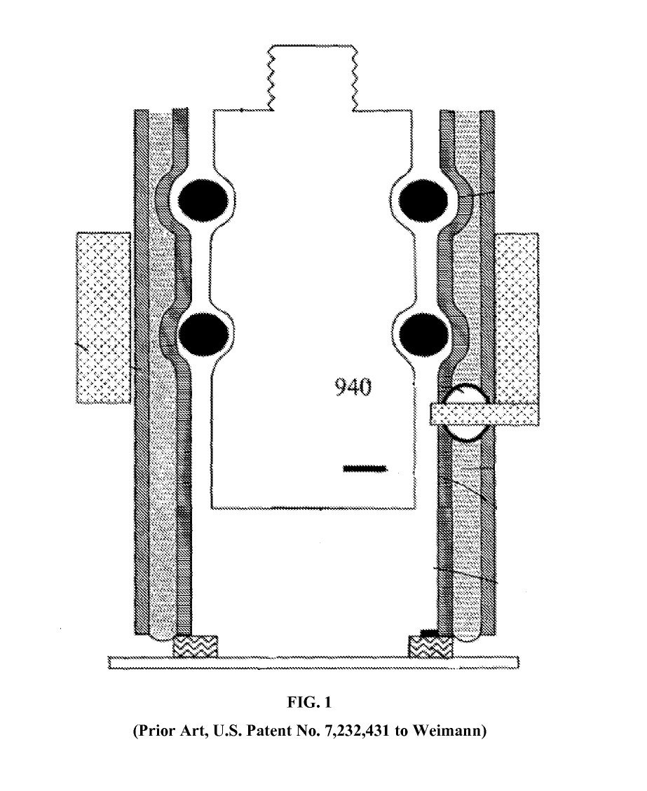

FIG. 1, is an illustration of an exemplary immersion ultrasound transducer described in U.S. Pat. No. 7,232,431 to Weimann, which can be used in the cleaning step. When the ultrasonic device is filled for cleaning, the tip of the ultrasonic horn is immersed into the solvent. In one embodiment, the ultrasonic horn is in direct contact with the skin. In a preferred embodiment, the ultrasonic horn is partially immersed in the solvent. When the horn is partially immersed in the solvent, the tip of the horn is about 1 mm to about 20 mm above the skin surface, preferably, about 5 mm to about 15 mm above the skin surface.

The frequency of the ultrasound is typically less than 1 MHz, preferably up to 100 kHz since the threshold for cavitation occurs at lower energies for lower frequencies. Preferably the frequencies range from about 20 kHz to about 100 kHz, more preferably from about 20 kHz to 60 kHz. The typical intensity of ultrasound is in the range of 0.1 and 100 W/cm², more typically between 1 W/cm² and 30 W/cm². Exposure time, defined as the time during which ultrasound is turned on is typically for between 1 s and 10 minutes, preferably between 2 s and 5 minutes, preferably between 5 s and 1 min. Both pulsed and continuous operations are possible, with preference for continuous to make the treatment faster.

B. Delivering Nanoshell Particles to the Sebaceous Glands

Delivery of the nanoshell particles to the sebaceous glands can be achieved by any suitable means, including but are not limited to low frequency ultrasound, the combination of low frequency ultrasound with high frequency ultrasound, iontophoresis, ultrasound, electroporation, injection, liposome encapsulation technology, other means for delivery of nanoparticles into the dermal region of the skin, e.g., pharmaceutically acceptable carriers, or combination thereof. Preferably, an ultrasonic technology is used to deliver the nanoshell composition. The ultrasound assists in propelling the nanoshell particles through the infundibula, penetrating the sebaceous glands.

Typically the step of delivering the nanoshells to the sebaceous glands occurs only once in the method. However, optionally, the step of delivering the nanoshells to sebaceous glands may be repeated, repeated two times, three times, or even up to five times at a site on the skin.

a. Applicator

Any suitable ultrasound apparatus can be used for delivery of the nanoshell particles in a suspension. Preferably, the apparatus is able to move from a first area of the skin in need of treatment, to a second area of the skin, without breaking the seal between the skin and the ultrasonic device. In one embodiment, the ultrasonic device has a pump for filling and emptying the reservoir. Preferably the applicator is a handheld device. An exemplary ultrasound transducer is described in U.S. Pat. No. 7,232,431 to Weimann (see FIG. 1).

The ultrasonic device for delivery of the nanoshell particles may include an ultrasound horn having a tip submerged in the nanoshell suspension and applying ultrasound radiation to the nanoshell suspension wherein the ultrasound radiation is applied at a frequency, an intensity, for a period of time, and at a distance from the skin, effective to generate cavitation bubbles, wherein the cavitation bubbles collapse causing microjets. The microjets drive particles into the follicles.

b. Ultrasound Frequency

The ultrasound is typically applied to the nanoshell suspension at a frequency less than 1 MHz, preferably ranging from 1 kHz to 1 MHz, more preferably from 20 kHz to 60 kHz since the cavitation bubbles collapse is strong in this frequency range. The intensity should not be so high as to raise the skin temperature more than about one to two degrees Centigrade.

A sufficient portion of the nanoshells remain intact during delivery into the infundibula and sebaceous glands to allow the energy (light) source, e.g., a laser to selectively thermally activate the nanoshell particles and thereby heat up the infundibula and sebaceous glands. Preferably the majority of the nanoshells are delivered into the infundibula and sebaceous glands without rupturing. More preferably substantially all of the nanoshells are delivered intact.

The ultrasound radiation may be continuous or pulsed and it may be applied for a period of time in the range of about 1 second and 10 minutes, preferably between 2 seconds and 5 minutes, more preferably between 5 seconds and 1 minute, but may be shorter and/or pulsed.

The ultrasound modalities are suitable to push the nanoshell particles into the infundibula and sebaceous glands without damaging the surrounding skin, the follicle root, or any other tissue surrounding the hair follicle.

a. Nanoshell Particles

Metal nanoshells are delivered through the hair follicles to the sebaceous glands. Metal nanoshells are a type of “nanoparticle” composed of a non-conducting, semiconductor or dielectric core coated with an ultrathin metallic layer. The diameter of a nanoshell particles ranges from about 50 nm to about 1 μm.

Metal nanoshells have unique physical properties. Specifically, metal nanoshells possess optical properties similar to metal colloids, i.e., a strong optical absorption and an extremely large and fast third-order nonlinear optical (NLO) polarizability associated with their plasmon resonance. A review of metal nanoshells and methods for making them are provided in Hirsch et al, Annals. of Biomedical Engineering, 2006, 34:15-22; Loo et al., Technology in Cancer Research and Treatment, 2004, 3:33-40; and U.S. Pat. No. 6,699,724 to West et al., the pertinent portions of which are incorporated herein by reference.

The nanoshell particles are constructed with a core diameter to shell thick ratio ranging from about 0.1-2. This ratio range coupled with control over the core size results in a particle that has a large, frequency-agile absorbance over most of the visible and infrared regions of the spectrum. The nanoshell particles preferably absorb thermal energy in an absorption spectrum in the range of 700-1100 nm. This minimizes surrounding blood from absorbing light intended for the material (hemoglobin absorbs most strongly at the violet end of the spectrum).

i. Metal Shell

Suitable metals for forming the shell or outer layer of the nanoparticle include the noble and coinage metals, but other electrically conductive metals may also be employed. Metals that are particularly well suited for use in shells include, but are not limited to, gold, silver, copper, platinum, palladium, lead, iron, and the like, or combinations thereof. Preferably, the shell is made from gold or silver. Alloys or non-homogenous mixtures of such metals may also be used. The shell layer is preferably about 1 nm to about 100 nm thick and coats the outer surface of the core uniformly.

ii. Nanoshell Core

The core is preferably made from a non-conducting or dielectric material. Suitable dielectric core materials include, but are not limited to, silicon dioxide, gold sulfide, titanium dioxide, polymethyl methacrylate (PMMA), polystyrene, and macromolecules such as dendrimers, or combinations thereof. The dielectric constant of the core material affects the absorbance characteristics of the overall particle. The core may be a mixed or layered combination of dielectric materials. The core may have a spherical, cubical, cylindrical or other shape.

Preferably, the nanoshell core is substantially homogeneous in size and shape, and preferably spherical. The shell core is preferably about 50 nm to about 500 nm thick and, depending upon the desired absorbance maximum of the particles.

iii. Nanoshell Surface

The nanoshell particles optionally contain a surface-bonding agent, effective to prevent aggregation of the nanoparticles. Preferably, the surface-bonding agent interacts with the nanoparticles to provide an organic layer surrounding the nanoparticles. In a preferred embodiment, the surface-bonding agent is a carboxylic acid, aldehyde, amide, alcohol, or a polyethylene glycol polymer. More preferably, the surface-bonding agent is a polyethylene glycol polymer. The outer layer is preferably about 1 to about 100 nm thick and coats the outer surface of the metal shell uniformly.

In one embodiment, the nanoshell particles can be formulated into a neutral, anionic, or cationic form. Suitable cationic and anionic groups include, but are not limited to, organic acids such as acetic, oxalic, tartaric, mandelic, and/or the like; polymers such as poly(sodium 4-styrenesulfonate), and poly(allylamine hydrochloride).

Optionally, the nanoshell particles further contain a therapeutic agent to be delivered into the sebaceous gland. Suitable therapeutic agents include, but are not limited to, salicylic acid, benzoyl peroxide, sulfur, retinoic acid, azelaic acid, clindamycin, adapalene, erythromycin, sodium sulfacetamide, aluminium chloride, resorcinol, dapsone, aluminum oxide, and combinations thereof. The therapeutic agent can be attached to the surface of the nanoshell particle by any suitable means.

v. Size and Shape of Nanoshell

The nanoshell particles may be homogenous or heterogeneous in size. Preferably, the particles are substantially homogeneous in size and shape, and preferably spherical. Where optimal plasmonic resonance is desired, the size of the nanoparticles is generally about 50 nm to about 500 nm, preferably from about 100 nm to about 250 nm, at least in one dimension.

The nanoshell particles preferably contain a silica core, a gold shell layer, and an outer layer of polyethylene glycol. The wavelength of maximum optical absorption (λₘₐₓ) of the particle is determined by the ratio of the core radius to the shell thickness for a particle of given core and shell materials and particle diameter. Each of these variables (i.e., core radius and shell thickness) can be easily and independently controlled during fabrication of the nanoshells. Varying the shell thickness, core diameter, and the total nanoparticle diameter allows the optical properties of the nanoshells to be tuned over the visible and near-IR spectrum, as described and illustrated in more detail in U.S. Pat. No. 6,344,272 to Oldenburg et al. By also varying the core and shell materials, which are preferably gold or silver over a silica core, the tunable range can be extended to cover most of the UV to near-infrared spectrum. Thus, the optical extinction profiles of the nanoshells can be modified so that the nanoshells optimally absorb light emitted from various lasers.

b. Carriers

Typically, the nanoshell particles are prepared as liquid solutions and/or suspensions and/or emulsion. Preferably, the nanoshell particles are prepared as a suspension. The nanoshell suspensions contain from about 10⁹ to about 10¹⁶ nanoshells per mL. Preferably, the suspensions contain from about 10¹⁰ to about 10¹³ nanoshells per mL.

In addition to the nanoshell particles, the suspensions may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, glycerol, tetrahydrofuryl alcohol, polyethylene glycols and fatty acid esters of sorbitan, and mixtures thereof.

In one embodiment, the nanoshell suspensions may also contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

i. Liposomes

Optionally, the nanoshells may be encapsulated within a liposome. Liposomes are microscopic spherical membrane-enclosed vesicles or sacks (0.5-500 μm in diameter) made artificially in the laboratory using a variety of methods. The liposomes are non-toxic to living cells and selected to deliver the nanoshell particles into the follicle and immediately surrounding tissue. A general discussion of the liposomes and liposome technology can be found in an article entitled, “Liposomes” by Marc J. Ostro, published in Scientific American, January 1987, Vol. 256, pp. 102-111 and in “Liposome Technology” edited by G. Gregorriadis, 1984, published by CRC press, Boca Raton, Fla. the pertinent portions of which are incorporated herein by reference.

C. Thermal Activation of the Nanoshell Particles

Following administration of the nanoparticles to the sebaceous glands, an energy (light) source, e.g., a laser, is matched with a wavelength to the absorption spectrum of the nanoshell particle to selectively thermally activate the nanoshell particles. When the nanoshell particles are activated, they heat up, and the heat is transferred to the surrounding tissue.

The thermal degradation of the sebaceous glands modifies the pore opening to the infundibulum such that the geometry, e.g., the shape, of the opening is permanently altered. The constriction, closure, or opening of the pore prevents accumulation of dirt, oils, bacteria, or viruses in that follicle. The opening to the infundibulum may be altered such that pore blockage, resulting in a blackhead or white head, will not occur. Alternately, the opening to the infundibulum may be opened. Preferably, the sebaceous glands are destroyed, thereby preventing the reoccurrence of acne. However, there is minimal to no destruction of normal adjacent epidermal and dermal structures.

a. Energy Source

Preferably, the energy source produces a large area of radiation to treat areas of skin afflicted with a sebaceous gland disorder. Alternately, the energy source is easily maneuverable to treat more than one adjacent areas of the skin afflicted with a sebaceous gland disorder.

Suitable energy sources include, but are not limited to, light-emitting diodes, incandescent lamps, xenon arc lamps, lasers or sunlight. Representative examples of continuous wave apparatus include, for example, diode lasers and light emitting diodes. A laser may also be used as a continuous wave apparatus. Suitable examples of pulsed lasers include, for example pulsed Nd:YAG lasers and Alexandrite lasers.

b. Energy Emitted from the Energy Source at the Skin Site

The energy emitted by the energy source is limited such that the skin is not damaged or absorption by the surrounding blood while the sebaceous gland disorder is treated. Hemoglobin absorbs most strongly at the violet end of the spectrum. For example, at 755 nm, up to 100 J/cm² can be administered to a very fair Caucasian individual without damage to the skin. The amount of energy a darker skin could tolerate without damage to the skin would be less. One of skill in this art can ascertain the amount of energy and type of energy to be expended to achieve the results desired. Typically, the energy source emits a wavelength ranging from about 750 nm to about 1100 nm.

The depth of penetration of the energy emitted from the energy source is dependent upon its wavelength. Wavelengths in the visible to near IR have the best penetration and are therefore best for use to thermally activate the nanoshells within the sebaceous gland and infundibulum.

Thermal activation of the nanoshell particles can be pulsed or continuous to facilitate temperature rise. The pulse duration time period should be shorter than that of the thermal relaxation time for the target, e.g., sebaceous gland. The thermal relaxation time is defined as the time it takes for a structure to cool to 50% of its peak temperature immediately following exposure to a light source capable of providing enough energy to photoactivate the nanoparticle. The energy deposited by a pulse that is shorter than the thermal relaxation time of the particle instantaneously minimizes heat diffusion. Therefore, treatment of the dermal regions containing a nanoshell particle will occur when exposed to millisecond light pulses. A laser delivering pulses in the range of 1 to 500 milliseconds (ms) can heat the infundibulum and sebaceous gland.

A continuous laser would have to be scanned so that the dwell time is less than the thermal relaxation time of the target.

Although the methods disclosed herein generally refer to the use of ultrasound to clean the skin site and to deliver the nanoshell particles into the sebaceous gland, alternatively or additionally an electric field, such as iontophoresis or electroporation, could be applied during the cleaning step and/or the delivery step.

### EXAMPLES

### Comparison of Different Methods for Delivering Nanoshell Particles to Sebaceous Gland Followed by Laser Therapy

A. Delivery Via Massage

Fresh, in vitro human sebaceous skin samples were used. F78 Sebashell™ (120 nm silica core, 30 nm gold shell, 30 nm PEG outer layer) suspensions (24% water, 54% 100 proof ethanol, 20% diisopropyl adipapte, 1% polysorbate 80) were applied three or four times to the skin samples, up to a total of 1.0 mL. The Sebashells™ were massaged into the skin for a period of four (4) minutes. The skin was then wiped to remove excess residual suspension from the skin surface. A 30 ms laser pulse, wavelength of 800 nm, about 50 J/cm², was used to thermally activate the nanoshell particles.

Skin was cut perpendicular to the top surface, preferably through a follicle and the vertical cross-section was observed under a dissecting microscope. Multiple such cuts were done and the fraction of infindibuli and sebaceous glands damaged were noted.

Results:

Penetration of the nanoshell particles in the infundibula was observed in less than half of the infundibula seen. Also, shallow and rare penetration of the sebaceous gland was observed.

B. Delivery Via Iontophoresis

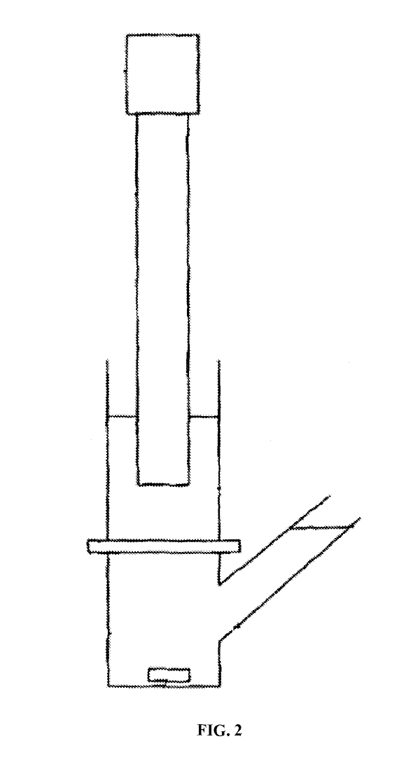

In a Franz Cell apparatus shown in FIG. 2, 0.5 mL of a 1% a F78 Sebashell™ suspension was placed in the donor compartment over the human cadaver epidermis. The experiment was carried out using poly(sodium 4-styrenesulfonate) coated nanoshells (negative charge, PS-1). The receiver compartment was filled with a saline solution.

The skin was mounted on the diffusion cell and then exposed to iotophoresis. In one instance, the skin was exposed to iontophoresis without massage. In a second instance, the skin was exposed to iotophoresis, then massaged before exposure to laser.

A 30 ms laser pulse, wavelength of 800 nm, about 50 J/cm², was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:
Iontophoresis Only:

Mid-level penetration of less than half infundibulum with PS-1 was observed. Furthermore there was shallow and rare penetration of the sebaceous gland.
Iontophoresis and Massage:

Deeper levels of penetration of less than half infundibulum with PS-1 compared to iontophoresis without massage was observed.

C. Delivery Via Ultrasound

A variety of different ultrasound modalities were tested. Appendix A contains two Tables which summarize the different test conditions and results. Averages are also provided in these Tables. Table 2 is generally identical to Table 3, however Table 3 contains columns B-E (entitled “Freq.”, “Transducer”, “Total time”, and “Duty cycle”, respectively) while these columns are not visible in Table 2.

In each set of Test conditions the nanoshell particles delivered were Sebashells™ (120 nm silica core, 30 nm gold shell, 30 nm PEG outer layer).

Abbreviations

Abbreviations used throughout the Table in Appendix are defined below.

FDC: Franz Diffusion Cell

Cont: Continuous Ultrasound Pulse. The total time is given in column D, entitled “Total Time”. Except when specified using the term “cont”, the ultrasound wave was pulsed.

Ace: Acetone

DMSO: Dimethyl Sulfoxide

SS: Sebashell™ Particles (120 nm silica core, 30 nm gold shell, 30 nm PEG outer layer)

ABS: Acrylonitrile Butadiene Styrene.

THC: Transducer Holding Cup manufactured from ABS by Catapult

Freq.: Frequency

SG: Sebaceous Gland

Imm.: Immersion Ultrasound. The ultrasound horn was immersed in the solvent and/or suspension at a distance away from the skin. The distance is provided in column H, entitled “Distance”.

OD: Optical Density

Al: Aluminium.

DIA: Diisopropyl adipate

H2O: Water

33C: Temperature is 33° C.

Columns

Each of the columns in the Tables in Appendix A is briefly described below.

Column A, entitled “Description”: Brief description of the apparatus the experiment was carried out with, and/or the ultrasound modalities, and/or the cleaning solvent used.

Column B, entitled “Frequency”: Frequency of the ultrasound device.

Column C, entitled “Transducer”: The diameter of the transducer probe.

Column D, entitled “Total Time”: The time ultrasound is immersed in the solvent or suspension or in direct contact with the epidermis.

Column E, entitled “Duty Cycle”: The ratio of “pulse on” to “pulse off” during a treatment is referred to as “duty cycle”. A 100% duty cycle is the same as “continuous”.

Column F, entitled “Amplitude”: A measure of the horn surface amplitude at the surface.

Column G, entitled “Repeat”: The number of times the experiment described in the first column was repeated.

Column H, entitled “Distance”: The distance between the tip of the ultrasound horn and the skin.

Column I, entitled “SS Volume”: Refers to the volume of nanoshell particles delivered to the follicle. The nanoshell particles delivered into the follicle. The Sebashell™ particles have a 120 nm silica core, 30 nm gold shell, and a 30 nm PEG outer layer.

Column J, entitled “Laser”: Irradiation of the nanoshells with a 30 ms laser pulse, wavelength of 800 nm, about 50 J/cm².

Column K, entitled “Total Rate”: The percent of infundibula damaged after irradiation of the nanoshell particles with a 30 ms laser pulse, wavelength of 800 nm, about 50 J/cm².

Column L, entitled “Std. Dev.”: Standard deviation of the average total rate.

Column M, entitled “SG Rate”: The percent of sebaceous gland damaged after irradiation of the nanoshell particles with a 30 ms laser pulse, wavelength of 800 nm, about 50 J/cm².

Column N, entitled “Std. Dev.”: Standard deviation of the average SG rate.

Column o, entitled “Deep SG”: Deep penetration of the nanoshell particles into the sebaceous gland. Deep penetration of the sebaceous glands can be determined by observation under a microscope, such as described in the Examples. Deep penetration as used herein refers to sebaceous glands that showed significant damage following thermal irradiation of nanoshell particles within the glands.

Column P, entitled “Std. Dev.”: Standard deviation of the average Deep SG.

Column Q, entitled “Max Temp”: The maximum temperature of the Sebashell™ suspension during sonication of the suspension.

Column R, entitled “Additional Description/Comments”: Gives additional description of how the experiments were carried out.

Experiments:

Test 1: 20 kHz Ultrasound—No Cleaning Step

(See rows 3 to 21 in the Table). In one exemplary embodiment, the experiment was carried out in a Franz Cell apparatus. 0.5 mL of a F78 Sebashell™ suspension was placed in the donor compartment over the human cadaver epidermis. The receiver compartment was filled with a saline solution.

An ultrasound horn was submerged in the nanoshell suspension at 2.5 mm or 5 mm height above the skin surface or in direct contact (0 mm) with the skin (see Appendix, column H entitled “Distance”).

The ultrasound, 20 kHz frequency, was turned on for periods of time indicated in the Appendix, column D entitled “total time”.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:

Direct Contact:

When the ultrasound horn was in direct contact with the skin, no penetration of the infundibula or the sebaceous gland was observed.

Immersion:

Ultrasound horn is 2.5 mm above the skin surface: Rare penetration of the sebaceous gland was observed.

Ultrasound horn is 5 mm above the skin surface: Penetration of about half infundibulum was observed for both pulsed and continuous wave ultrasound. Only shallow and rare penetration of the sebaceous gland was observed of pulsed wave ultrasound, and on average only 17% showed deep penetration.

Test 2: 20 kHz Ultrasound—with Cleaning Step Before Delivery of Nanoshell Particles (See Rows 22-38 and 43-53 in the Appendix).

In one exemplary embodiment, the experiment was carried out in a Franz Cell apparatus. Various cleaning solvents, including acetone, ethanol, water, isopropanol, and DMSO, were placed in the donor compartment of the FDC apparatus. The receiver compartment was filled with a saline solution.

An ultrasound horn was submerged in the nanoshell suspension at 5 mm height above the skin surface or in direct contact with the skin (see Appendix, column H entitled “Distance”).

The ultrasound, 20 kHz frequency, was turned on for periods of time indicated in the Appendix, column D entitled “total time”.

The donor compartment of the apparatus was emptied and refilled with 0.5 mL of a F78 Sebashell™ suspension.

As before, the ultrasound horn was submerged in the nanoshell suspension at 5 mm height above the skin surface or in direct contact with the skin.

The ultrasound, 20 kHz frequency, was turned on for periods of time indicated in the Appendix, column D entitled “total time”.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done, and the fraction of infundibula and sebaceous glands damaged were noted.

The experiment was repeated with a 130 W ultrasonic device (see row 65).

Results:

Direct Contact:

Penetration of about half the infundibula was observed. Penetration of over a third of the sebaceous glands, of which about a third was deep sebaceous gland penetration.

Immersion:

Penetration of greater than half infundibulum (up to 80%) was observed. Penetration of greater than a third of the sebaceous gland, about a quarter was deep penetration.

Test 3: 20 kHz Ultrasound—with Cleaning Simultaneous with Delivery of Nanoshell Particles (See Rows 39-42 and 54-60 in the Appendix).

In one exemplary embodiment, the experiment was carried out in a Franz Cell apparatus. 0.5 mL of a F78 Sebashell™ suspension with the cleaning solvent (24% water, 54% 100 proof ethanol, 20% diisopropyl adipapte, 1% polysorbate 80) was placed in the donor compartment of the FDC apparatus. The receiver compartment was filled with a saline solution.

An ultrasound horn was submerged in the nanoshell suspension at 5 mm height above the skin surface or in direct contact with the skin (see Appendix, column H entitled “Distance”).

The 600 W ultrasonic device, 20 kHz frequency, was turned on for periods of time indicated in the Appendix, the column D entitled “total time”.

The experiment was repeated once (rows 39-42) or two times with a fresh Sebashell™ suspension (rows 54-57) or two times with the first Sebashell™ suspension recycled (rows 58-60).

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

The experiment was repeated with a 130 W ultrasonic device (see rows 61-64).

Results:

Immersion:

Penetration of almost all the infundibulum (up to 94%) was observed. Penetration of greater than 60% of the sebaceous gland, greater than 35% was deep penetration.

Test 4: 40 kHz Ultrasound—with Cleaning Step Simultaneous with Delivery of Nanoshell Particles (See Rows 83-113 in the Appendix).

In one exemplary embodiment, the experiment was carried out in a Franz Cell apparatus. 0.5 mL of a F78 Sebashell™ suspension with the cleaning solvent (24% water, 54% 100 proof ethanol, 20% diisopropyl adipapte, 1% polysorbate 80) was placed in the donor compartment of the FDC apparatus. The receiver compartment was filled with a saline solution.

An ultrasound horn was submerged in the nanoshell suspension at 5 mm or 8 mm height above the skin surface (see Appendix, column H, entitled “Distance”).

The 130 W ultrasonic device, 40 kHz frequency, was turned on for periods of time indicated in the Appendix, column D, entitled “total time”. The amplitude of the ultrasonic device is as described in column F, entitled “Amplitude”.

The experiment was repeated once.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:

Immersion:

Penetration of greater than three quarters of the infundibulum was observed. Penetration of less than half the sebaceous glands, of which less than half was deep penetration.

Test 5: 20 kHz or 40 kHz Ultrasound—Modified Ultrasound Apparatus (See Rows 114-141 in the Appendix).

In one exemplary embodiment, the experiment was carried out in an acrylonitrile butadiene styrene cup or an aluminium cup. 0.5 mL of a F78 Sebashell™ suspension with the cleaning solvent (24% water, 54% 100 proof ethanol, 20% diisopropyl adipapte, 1% polysorbate 80) was placed in the donor compartment of the transdermal holding cup (THC) made by acrylonitrile butadiene styrene, or aluminum. The cadaver epidermis was placed in the bottom of the cup.

An ultrasound horn was submerged in the nanoshell suspension at about 8 mm height above the skin surface (see Appendix, column H, entitled “Distance”).

The 130 W ultrasonic device, 20 kHz or 40 kHz frequency, was turned on for periods of time indicated in the Appendix, column D, entitled “total time”. The amplitude of the ultrasonic device is as described in column F, entitled “Amplitude”.

The experiment was repeated once.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:

The bottom of the ABS or Al cup reflected the sound wave of the ultrasound, therefore this experimental set-up did not work.

Test 6: 40 kHz Ultrasound—No Cleaning Step (See Rows 142-146 in the Appendix).

In one exemplary embodiment, the experiment was carried out in a Franz Cell apparatus. 0.5 mL of a F78 Sebashell™ suspension was placed in the donor compartment over the human cadaver epidermis. The receiver compartment was filled with a saline solution.

An ultrasound horn was submerged in the nanoshell suspension at about 8 mm height above the skin surface (see Appendix, column H, entitled “Distance”).

The ultrasound, 40 kHz frequency, was turned on for periods of time indicated in the Appendix, column D, entitled “total time”.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:

Immersion:

Penetration of greater than three quarters of the infundibulum was observed. Penetration of about half the sebaceous glands, of which less than a quarter was deep penetration.

Test 7: 40 kHz Ultrasound—Sebashell™ Suspension without Diisopropyl Adipate (See Rows 147-149 in the Appendix).

In one exemplary embodiment, the experiment was carried out in a Franz Cell apparatus. 0.5 mL of a F78 Sebashell™ suspension (as described in Test 1 without diisopropyl adipate) with the cleaning solvent was placed in the donor compartment of the FDC apparatus. The receiver compartment was filled with a saline solution.

An ultrasound horn was submerged in the nanoshell suspension at about 8 mm height above the skin surface (see Appendix, column H, entitled “Distance”).

The 130 W ultrasonic device, 40 kHz frequency, was turned on for periods of time indicated in the Appendix, column D, entitled “total time”. The amplitude of the ultrasonic device is as described in column F, entitled “Amplitude”.

The experiment was repeated once.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:

Immersion:

Penetration of greater than three quarters of the infundibulum was observed. Penetration of about half the sebaceous glands was observed.

Test 8: 40 kHz Ultrasound—Sebashell™ Suspension with OD 75 or 125 (See Rows 150-159 and 193-195 in the Appendix).

In one exemplary embodiment, the experiment was carried out in a Franz Cell apparatus. A F78 Sebashell™ suspension with an OD of 75 or 125 (see description of rows 147-159) was placed in the donor compartment of the FDC apparatus. The receiver compartment was filled with a saline solution.

An ultrasound horn was submerged in the nanoshell suspension at 8 mm or 8 mm height above the skin surface (see Appendix, column H, entitled “Distance”).

The 130 W ultrasonic device, 40 kHz frequency, was turned on for periods of time indicated in the Appendix, column D, entitled “total time”. The amplitude of the ultrasonic device is as described in column F, entitled “Amplitude”.

The experiment was repeated once.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:

Immersion:

Penetration of greater than three quarters of the infundibulum was observed. For some of the conditions tested, penetration of about half the sebaceous glands, of which about a quarter was deep penetration.

Test 9: 40 kHz—with a Modified Ultrasound Apparatus (See Rows 160-192 in the Appendix).

Various test were carried out to test the reflective properties at the interface of the skin and subdermal tissue. The experiments were carried out in a transducer holding cup (such as used in Test 5) with various materials (also referred to as “receivers”) interfacing the skin. The receivers used are described in column A of rows 160-192 and included aluminium plate, water, air, beef T-bone with and without cartilage, and pig bone with and without cartilage.

Results:

The reflecting properties of the materials at the interface of the skin made a difference in the penetration of the nanoshell particles into the infundibula and hair follicles as seen in the results. For example, when the interface is aluminium, penetration of less than half the infundibula was observed. However, when water is at the interface of the skin, penetration of about 90% nanoshell particles into the infundibula was observed.

Test 10: 40 kHz Ultrasound—Delivery of Nanoshell Particles at 20 mm Horn Diameter (See Rows 196-204 in the Appendix).

In one exemplary embodiment, the experiment was carried out in a Franz Cell apparatus. 0.5 mL of a F78 Sebashell™ suspension with the cleaning solvent (24% water, 54% 100 proof ethanol, 20% diisopropyl adipapte, 1% polysorbate 80) was placed in the donor compartment of the FDC apparatus. The receiver compartment was filled with a saline solution.

An ultrasound horn was submerged in the nanoshell suspension at about 8 mm height above the skin surface (see Appendix, column H, entitled “Distance”).

For the cleaning step: The 130 W ultrasonic device, 40 kHz frequency, was turned on for periods of time indicated in the Appendix, column D, entitled “total time”. The amplitude of the ultrasonic device is as described in column F, entitled “Amplitude”.

For the delivery of the nanoshell particles: The 130 W ultrasonic device, 40 kHz frequency, was turned on for 60 seconds. The amplitude of the ultrasonic device is as described in the column F, entitled “Amplitude”.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:

Immersion:

For some experiments, penetration of 100% the infundibulum was observed. For some experiments, penetration of over 60% of the sebaceous glands, of which about 40% was deep penetration.

Test 11: 40 kHz Ultrasound—Modified Ultrasound Horn (See Rows 205-210 in the Appendix).

In one exemplary embodiment, the experiment was carried out in a Franz Cell apparatus. 0.5 mL of a F78 Sebashell™ suspension with the cleaning solvent (24% water, 54% 100 proof ethanol, 20% diisopropyl adipate, 1% polysorbate 80) was placed in the donor compartment of the FDC apparatus. The receiver compartment was filled with a saline solution.

An ultrasound horn was submerged in the nanoshell suspension at between 8 mm or 15 mm height above the skin surface (see Appendix, column H, entitled “Distance”).

The 130 W ultrasonic device, 40 kHz frequency, was turned on for periods of time indicated in the Appendix, column D, entitled “total time”. The amplitude of the ultrasonic device is as described in column F, entitled “Amplitude”. The diameter of the horn is as described in column A, entitled “description”.

The skin was cleaned with a cloth to remove excess Sebashell™ particles from the surface.

A 30 ms laser pulse, wavelength of 800 nm (corresponding to about 50 J/cm²), was used to thermally activate the nanoshell particles.

The skin was cut perpendicular to the top surface, preferably through a follicle and the vertical cross-section was observed under a dissecting microscope. Multiple such cuts were done and the fraction of infundibula and sebaceous glands damaged were noted.

Results:

Immersion:

Penetration of greater than three quarters of the infundibulum was observed. Penetration of about half the sebaceous glands, of which less than a quarter was deep penetration.

TABLE 1
Summary
Penetration of the infundibula and sebaceous
glands using 20 kHz ultrasound.

| | IF | SG | Deep SG | |
|---|---|---|---|---|
| Description | penetration^{a} | penetration^{a} | penetration^{c} | Conditions |
| No precleaning | 54.9 (1.8) | 21.9 (4.4) | 17.0 (6.3) | 1 min (100% |
| step | | | | duty cycle, |
| | | | | once) |
| Preclean with | 64.5 (8.7) | 44.3 (10.6) | 20.9 (14.3) | 1 min (100% |
| Acetone | | | | duty) |
| Preclean with | 70.8 (4.6) | 41.3 (8.6) | 17.9 (4.9) | 1 min (100% |
| isopropanol | | | | duty) |
| Preclean with | 60.0 (19.5) | 38.0 (16.4) | 14.0 (3.5) | 1 min (100% |
| water | | | | duty) |
| Preclean with | 67.7 (14.0) | 23.3 (14.0) | 13.7 (10.5) | 1 min (100% |
| ethanol | | | | duty) |
| Preclean with | 61.5 (2.1) | 39.0 (8.5) | 3.6 (5.1) | 50 sec |
| DMSO | | | | (100% duty) |
| | | | | (45 C.) |
| Repeated | 82.7 (9.5) | 49.7 (13.3) | 31.1 (17.1) | 1 min (100% |
| application of | | | | duty, ×2) |
| Sebashell ™ | | | | |
| (×2) | | | | |
| Repeated | 94.3 (1.5) | 65.3 (28.1) | 39.1 (10.0) | 1 min (100% |
| application of | | | | duty, ×3) |
| Sebashell ™ | | | | |
| (×3) | | | | |
| Repeated | 87.0 (5.7) | 66.5 (10.6) | 35.5 (5.2) | 1 min (100% |
| application of | | | | duty, ×3) |
| Sebashell ™ | | | | |
| (×3, recycle) | | | | |

^{a}percent of the infundibular epidermis penetrated by nanoshells
^{b}percent of the sebaceous gland penetrated by nanoshells
^{c}percent of the sebaceous gland with deep penetration
Standard deviations are given in parenthesis

### SUMMARY

The results of this study demonstrated that the application of continuous ultrasound with a cleaning step (either before or during application of the nanoshell particles) significantly improved sebaceous gland penetration of the nanoshell (up to three times) compared to delivery of nanoshell without a cleaning step.

Furthermore, the results show ultrasound modalities/parameters that are suitable to push the nanoshell particles into the infundibula and sebaceous glands without damaging the surrounding skin, the follicle root, or any other surrounding tissue.

APPENDIX A TABLE 2

| | A | F | G | H | I | J | K | L |
|---|---|---|---|---|---|---|---|---|
| 1 | Description | Amplitude | Repeat | Distance | SS Volume | Laser | Total rate | Std. Dev. |
| 2 | Massage | weak | 3 | 0 mm | 1.0 ml | 50 J/cm2, 30 ms | | |
| 3 | FDC Immersion | 10% | 1 | 5 mm | 1.5 ml | 50 J/cm2, 30 ms | | |
| 4 | FDC Immersion | 10% | 4 | 5 mm | 1.5 ml | 50 J/cm2, 30 ms | | |
| 5 | FDC Immersion | 10% | 2 | 5 mm | 1.5 ml | 50 J/cm2, 30 ms | 46.7 | |
| 6 | FDC Immersion | 10% | 1 | 5 mm | 1.5 ml | 50 J/cm2, 30 ms | 60 | |
| 7 | FDC Immersion | 10% | 1 | 5 mm | 1.5 ml | 50 J/cm2, 30 ms | 61 | |
| 8 | FDC Immersion | 20% | 2 | 5 mm | 1.5 ml | 50 J/cm2, 30 ms | | |
| 9 | FDC Immersion | 30% | 2 | 5 mm | 1.5 ml | 50 J/cm2, 30 ms | | |
| 10 | FDC Immersion | 10% | 4 | 2.5 mm | 1.5 ml | 50 J/cm2, 30 ms | | |
| 11 | Direct contact | 10% | 3 | 0 mm | 1.0 ml | 50 J/cm2, 30 ms | | |
| 12 | FDC Immersion(cont, 130 W) | 20% | 1 | 5 mm | 1.5 ml | 50 J/cm2, 30 ms | 61.9 | |
| 13 | FDC Immersion(cont, 130 W) | 20% | 1 | 5 mm | 1.7 ml | 50 J/cm2, 30 ms | 50.0 | |
| 14 | FDC Immersion(cont, 130 W) | 20% | 1 | 5 mm | 1.7 ml | 50 J/cm2, 30 ms | 61.9 | |
| 15 | Average of three rows above | 20% | 3 | 5 mm | 1.5 ml | 50 J/cm2, 30 ms | 57.9 | 6.9 |
| 16 | FDC Immersion(cont) | 10% | 1 | 5 mm | 1.5 ml | 50 J/cm2, 30 ms | 53.6 | |
| 17 | FDC Immersion(cont) | 10% | 1 | 5 mm | 1.5 ml | 50 J/cm2, 30 ms | 56.2 | |
| 18 | Average of two rows above | 10% | 2 | 5 mm | 1.5 ml | 50 J/cm2, 30 ms | 54.9 | 1.8 |
| 19 | FDC Immersion(cont) | 15% | 1 | 5 mm | 1.7 ml | 50 J/cm2, 30 ms | 77 | |
| 20 | FDC Immersion(cont) | 15% | 1 | 5 mm | 1.7 ml | 50 J/cm2, 30 ms | 55 | |
| 21 | Average of two rows above | 15% | 2 | 5 mm | 1.7 ml | 50 J/cm2, 30 ms | 66.0 | 15.6 |
| 22 | FDC Immersion(cont, 130 W) + Ace | 10% | 1 | 5 mm | 1.7 ml | 50 J/cm2, 30 ms | 65 | |
| 23 | FDC Immersion + Acetone | 10% | 1 | 0 mm | 1.5 ml | 50 J/cm2, 30 ms | 57.9 | |
| 24 | FDC Immersion + Acetone | 10% | 1 | 0 mm | 1.5 ml | 50 J/cm2, 30 ms | 56 | |
| 25 | FDC Immersion + Acetone | 10% | 1 | 0 mm | 1.5 ml | 50 J/cm2, 30 ms | 68.2 | |
| 26 | FDC Immersion + Acetone + Vacuum | 10% | 1 | 0 mm | 1.5 ml | 50 J/cm2, 30 ms | 60.9 | |
| 27 | FDC Immersion(cont) + Acetone | 15% | 1 | 5 mm | 1.7 ml | 50 J/cm2, 30 ms | 62 | |
| 28 | FDC Immersion(Ace, | 10% | 1 | 0 mm | 1.0 ml | 50 J/cm2, 30 ms | 65 | |
| 29 | FDC Immersion(cont) + Ace | 10% | 1 | 5 mm | 1.7 ml | 50 J/cm2, 30 ms | 70.0 | |
| 30 | FDC Immersion(cont) + Ace | 10% | 1 | 5 mm | 1.7 ml | 50 J/cm2, 30 ms | 71.0 | |
| 31 | FDC Immersion(cont) + Ace | 10% | 1 | 5 mm | 1.7 ml | 50 J/cm2, 30 ms | 65.0 | |
| 32 | FDC Immersion(cont) + Ace | 10% | 1 | 5 mm | 1.7 ml | 50 J/cm2, 30 ms | 52.0 | |
| 33 | Average of four rows above | 10% | 4 | 5 mm | 1.7 ml | 50 J/cm2, 30 ms | 64.5 | 8.7 |
| 34 | FDC Immersion(cont) + Isopropanol | 10% | 1 | 5 mm | 1.7 ml | 50 J/cm2, 30 ms | 70.0 | |
| 35 | FDC Immersion(cont) + Isopropanol | 10% | 1 | 5 mm | 1.7 ml | 50 J/cm2, 30 ms | 65.0 | |
| 36 | FDC Immersion(cont) + Isopropanol | 10% | 1 | 5 mm | 1.7 ml | 50 J/cm2, 30 ms | 76.0 | |
| 37 | FDC Immersion(cont) + Isopropanol | 10% | 1 | 5 mm | 1.7 ml | 50 J/cm2, 30 ms | 72.0 | |
| 38 | Average of four rows above | 10% | 4 | 5 mm | 1.7 ml | 50 J/cm2, 30 ms | 70.8 | 4.6 |
| 39 | FDC Immersion(cont) + Sebashell | 10% | 1 | 5 mm | 1.7 ml | 50 J/cm2, 30 ms | 86.0 | |
| 40 | FDC Immersion(cont) + Sebashell | 10% | 1 | 5 mm | 1.7 ml | 50 J/cm2, 30 ms | 72.0 | |
| 41 | FDC Immersion(cont) + Sebashell | 10% | 1 | 5 mm | 1.7 ml | 50 J/cm2, 30 ms | 90.0 | |
| 42 | Average of three rows above | 10% | 3 | 5 mm | 1.7 ml | 50 J/cm2, 30 ms | 82.7 | 9.5 |
| 43 | FDC Immersion(cont) + Water | 10% | 1 | 5 mm | 1.7 ml | 50 J/cm2, 30 ms | 79.0 | |
| 44 | FDC Immersion(cont) + Water | 10% | 1 | 5 mm | 1.7 ml | 50 J/cm2, 30 ms | 61.0 | |
| 45 | FDC Immersion(cont) + Water | 10% | 1 | 5 mm | 1.7 ml | 50 J/cm2, 30 ms | 40.0 | |
| 45 | Average of three rows above | 10% | 3 | 5 mm | 1.7 ml | 50 J/cm2, 30 ms | 60.0 | 19.5 |
| 47 | FDC Immersion(cont) + Ethanol | 10% | 1 | 5 mm | 1.7 ml | 50 J/cm2, 30 ms | 82.0 | |
| 48 | FDC Immersion(cont) + Ethanol | 10% | 1 | 5 mm | 1.7 ml | 50 J/cm2, 30 ms | 67.0 | |
| 49 | FDC Immersion(cont) + Ethanol | 10% | 1 | 5 mm | 1.7 ml | 50 J/cm2, 30 ms | 54.0 | |
| 50 | Average of three rows above | 10% | 2 | 5 mm | 1.7 ml | 50 J/cm2, 30 ms | 67.7 | 14.0 |
| 51 | FDC Immersion(cont) + DMSO | 10% | 1 | 5 mm | 1.7 ml | 50 J/cm2, 30 ms | 60.0 | |
| 52 | FDC Immersion(cont) + DMSO | 10% | 1 | 5 mm | 1.7 ml | 50 J/cm2, 30 ms | 63.0 | |
| 53 | Average of two rows above | 10% | 2 | 5 mm | 1.7 ml | 50 J/cm2, 30 ms | 61.5 | 2.1 |
| 54 | FDC Immersion(cont) + SS(twice) | 10% | 1 | 5 mm | 1.7 ml | 50 J/cm2, 30 ms | 96.0 | |
| 55 | FDC Immersion(cont) + SS(twice) | 10% | 1 | 5 mm | 1.7 ml | 50 J/cm2, 30 ms | 93.0 | |
| 56 | FDC Immersion(cont) + SS(twice) | 10% | 1 | 5 mm | 1.7 ml | 50 J/cm2, 30 ms | 94.0 | |
| 57 | Average of three rows above | 10% | 3 | 5 mm | 1.7 ml | 50 J/cm2, 30 ms | 94.3 | 1.5 |
| 58 | FDC Immersion(cont) + SS(twice, | 10% | 1 | 5 mm | 1.7 ml | 50 J/cm2, 30 ms | 83 | |
| | recycle) | | | | | | | |
| 59 | FDC Immersion(cont) + SS(twice, | 10% | 1 | 5 mm | 1.7 ml | 50 J/cm2, 30 ms | 91 | |
| | recycle) | | | | | | | |
| 60 | Average of two rows above | 10% | 2 | 5 mm | 1.7 ml | 50 J/cm2, 30 ms | 87.0 | 5.7 |
| 61 | FDC Immersion (cont, 130 W) + | 20% | 1 | 5 mm | 1.7 ml | 50 J/cm2, 30 ms | 91.3 | |
| | Sebashell | | | | | | | |
| 62 | FDC Immersion(cont, | 20% | 1 | 5 mm | 1.7 ml | 50 J/cm2, 30 ms | 100.0 | |
| | 130 W) + Sebashell | | | | | | | |
| 63 | FDC Immersion(cont, | 20% | 1 | 5 mm | 1.7 ml | 50 J/cm2, 30 ms | 95.0 | |
| | 130 W) + Sebashell | | | | | | | |
| 64 | FDC Immersion(cont, | 20% | 3 | 5 mm | 1.7 ml | 50 J/cm2, 30 ms | 95.4 | 4.4 |
| | 130 W) + Sebashell | | | | | | | |
| 65 | FDC Immersion(cont, | 20% | 1 | 5 mm | 1.7 ml | 50 J/cm2, 30 ms | 96.2 | |
| | 130 W) + Acetone | | | | | | | |
| 66 | FDC Immersion(cont, 130 W) + SS in | 20% | 1 | 5 mm | 1.7 ml | 50 J/cm2, 30 ms | 100.0 | |
| | 33 C. | | | | | | | |
| 67 | FDC Immersion(cont, 130 W) + SS in | 20% | 1 | 5 mm | 1.7 ml | 50 J/cm2, 30 ms | 100.0 | |
| | 33 C. | | | | | | | |
| 68 | FDC Immersion(cont, 130 W) + SS in | 20% | 1 | 5 mm | 1.7 ml | 50 J/cm2, 30 ms | 82.0 | |
| | 33 C. | | | | | | | |
| 69 | FDC Immersion(cont, 130 W) + SS in | 20% | 1 | 5 mm | 1.7 ml | 50 J/cm2, 30 ms | 80.0 | |
| | 33 C. | | | | | | | |
| 70 | FDC Immersion(cont, 130 W, | 20% | 1 | 5 mm | 1.7 ml | 50 J/cm2, 30 ms | 44.0 | |
| | human) in 33 C. | | | | | | | |
| 71 | FDC Immersion(cont, 130 W, | 20% | 1 | 5 mm | 1.7 ml | 50 J/cm2, 30 ms | 12.5 | |
| | human w/hair) in 33 C. | | | | | | | |
| 72 | FDC Immersion(cont, 600 W, | 10% | 1 | 5 mm | 1.7 ml | 50 J/cm2, 30 ms | 2/6 biopsies | |
| | human) | | | | | | | |
| 73 | Massage(rabbit ear) | weak | 1 | 0 mm | 1.0 ml | 50 J/cm2, 30 ms | | |
| 74 | FDC Immersion(cont, 130 W, | 20% | 1 | 5 mm | 1.7 ml | 50 J/cm2, 30 ms | | |
| | rabbit) | | | | | | | |
| 75 | FDC Immersion(cont, 130 W) + SS | 20% | 1 | 5 mm | 1.7 ml | 50 J/cm2, 30 ms | 100.0 | |
| 76 | FDC Immersion(cont, 130 W) + SS | 20% | 1 | 5 mm | 1.7 ml | 50 J/cm2, 30 ms | 78.0 | |
| 77 | Sonoprep Immersion(cont) + SS | 12 W RMS | 1 | 7.5 mm | 1.0 ml | 50 J/cm2, 30 ms | 77.0 | |
| 78 | Sonoprep Immersion(cont) + SS | 12 W RMS | 1 | 7.5 mm | 1.0 ml | 50 J/cm2, 30 ms | 93.0 | |
| 79 | Sonoprep Immersion(cont) + SS | 12 W RMS | 1 | 7.5 mm | 1.0 ml | 50 J/cm2, 30 ms | 36.0 | |
| 80 | Sonoprep Immersion(cont) + SS | 12 W RMS | 1 | 7.5 mm | 1.0 ml | 50 J/cm2, 30 ms | 75.0 | |
| 81 | FDC Immersion(cont, 130 W) + SS | 20% | 1 | 8 mm | 2.0 ml | 50 J/cm2, 30 ms | 100.0 | |
| 82 | FDC Immersion(cont, 130 W) + SS | 20% | 1 | 8 mm | 2.0 ml | 50 J/cm2, 30 ms | 90.0 | |
| 83 | FDC Immersion(cont, 130 W) + SS | 28% | 1 | 8 mm | 2.0 ml | 50 J/cm2, 30 ms | 85.7 | |
| 84 | FDC Immersion(cont, 130 W) + SS | 28% | 1 | 8 mm | 2.0 ml | 50 J/cm2, 30 ms | 100.0 | |
| 85 | FDC Immersion(cont, 130 W) + SS | 28% | 1 | 8 mm | 2.0 ml | 50 J/cm2, 30 ms | 100.0 | |
| 86 | Average of three rows above | 28% | 3 | 8 mm | 2.0 ml | 50 J/cm2, 30 ms | 95.2 | 8.2 |
| 87 | FDC Immersion(cont, 130 W) + SS | 20% | 1 | 8 mm | 2.0 ml | 50 J/cm2, 30 ms | 68.4 | |
| 88 | FDC Immersion(cont, 130 W) + SS | 20% | 1 | 8 mm | 2.0 ml | 50 J/cm2, 30 ms | 50.0 | |
| 89 | FDC Immersion(cont, 130 W) + SS | 20% | 1 | 8 mm | 2.0 ml | 50 J/cm2, 30 ms | 84.2 | |
| 90 | Average of three rows above | 20% | 3 | 8 mm | 2.0 ml | 50 J/cm2, 30 ms | 67.5 | 17.1 |
| 91 | FDC Immersion(cont, 130 W) + SS | 28% | 1 | 5 mm | 2.0 ml | 50 J/cm2, 30 ms | 100.0 | |
| 92 | FDC Immersion(cont, 130 W) + SS | 28% | 1 | 5 mm | 2.0 ml | 50 J/cm2, 30 ms | 84.2 | |
| 93 | Average of two rows above | 28% | 2 | 5 mm | 2.0 ml | 50 J/cm2, 30 ms | 92.1 | 11.2 |
| 94 | FDC Immersion(cont, 130 W) + SS | 28% | 1 | 5 mm | 2.0 ml | 50 J/cm2, 30 ms | 60.0 | |
| 95 | FDC Immersion(cont, 130 W) + SS | 28% | 1 | 5 mm | 2.0 ml | 50 J/cm2, 30 ms | 81.3 | |
| 96 | Average of two rows above | 20% | 2 | 5 mm | 2.0 ml | 50 J/cm2, 30 ms | 70.6 | 15.0 |
| 97 | FDC Immersion(cont, 130 W) + SS | 20% | 1 | 8 mm | 2.0 ml | 50 J/cm2, 30 ms | 66.7 | |
| 98 | FDC Immersion(cont, 130 W) + SS | 20% | 1 | 8 mm | 2.0 ml | 50 J/cm2, 30 ms | 86.4 | |
| 99 | Average of two rows above | 20% | 2 | 8 mm | 2.0 ml | 50 J/cm2, 30 ms | 76.5 | 13.9 |
| 100 | FDC Immersion(cont, 130 W) + SS | 28% | 1 | 8 mm | 2.0 ml | 50 J/cm2, 30 ms | 80.0 | |
| 101 | FDC Immersion(cont, 130 W) + SS | 28% | 1 | 8 mm | 2.0 ml | 50 J/cm2, 30 ms | 83.3 | |
| 102 | Average of two rows above | 28% | 2 | 8 mm | 2.0 ml | 50 J/cm2, 30 ms | 81.7 | 2.4 |
| 103 | FDC Immersion(cont, 130 W) + SS | 20% | 1 | 8 mm | 2.0 ml | 50 J/cm2, 30 ms | 75.0 | |
| 104 | FDC Immersion(cont, 130 W) + SS | 20% | 1 | 8 mm | 2.0 ml | 50 J/cm2, 30 ms | 100.0 | |
| 105 | Average of two rows above | 20% | 2 | 8 mm | 2.0 ml | 50 J/cm2, 30 ms | 87.5 | 17.7 |
| 106 | FDC Immersion(cont, 130 W) + SS | 28% | 1 | 8 mm | 2.0 ml | 50 J/cm2, 30 ms | 76.5 | |
| 107 | FDC Immersion(cont, 130 W) + SS | 28% | 1 | 8 mm | 2.0 ml | 50 J/cm2, 30 ms | 100.0 | |
| 108 | Average of two rows above | 28% | 2 | 8 mm | 2.0 ml | 50 J/cm2, 30 ms | 88.2 | 16.6 |
| 109 | Average of rows 83-85, 100-101, | 20% | 7 | 8 mm | 2.0 ml | 50 J/cm2, 30 ms | 75.8 | 16.2 |
| | and 106-107 above | | | | | | | |
| 110 | Average of rows 87-89, 97-98, | 28% | 7 | 8 mm | 2.0 ml | 50 J/cm2, 30 ms | 89.4 | 10.4 |
| | and 103-104 above | | | | | | | |
| 111 | FDC Immersion(cont, 130 W) + SS | 35% | 1 | 8 mm | 2.0 ml | 50 J/cm2, 30 ms | Sebashell | 66.7 |
| 112 | FDC Immersion(cont, 130 W) + SS | 35% | 1 | 8 mm | 2.0 ml | 50 J/cm2, 30 ms | 50.0 | |
| 113 | Average of two rows above | 35% | 2 | 8 mm | 2.0 ml | 50 J/cm2, 30 ms | 58.3 | 11.8 |
| 114 | THC Immersion(cont, 130 W), ABS, | 20% | 1 | 8 mm (10 mm) | 5.0 ml | 50 J/cm2, 30 ms | 61.1 | |
| | 20 mm | | | | | | | |
| 115 | THC Immersion(cont, 130 W) + SS, | 20% | 1 | 8 mm (10 mm) | 5.0 ml | 50 J/cm2, 30 ms | 67.6 | |
| | ABS, 20 mm | | | | | | | |
| 116 | 20 mm ABS Immersion(cont, | 28% | 1 | about 8 mm | 4.0 ml | 50 J/cm2, 30 ms | 72.0 | |
| | 130 W) + SS | | | | | | | |
| 117 | 18 mm ABS Immersion(cont, | 28% | 1 | about 8 mm | 3.2 ml | 50 J/cm2, 30 ms | 81.0 | |
| | 130 W) + SS | | | | | | | |
| 118 | 18 mm Al Immersion(cont, | 28% | 1 | about 8 mm | 3.2 ml | 50 J/cm2, 30 ms | 50.0 | |
| | 130 W) + SS | | | | | | | |
| 119 | 16 mm ABS Immersion(cont, | 28% | 1 | about 8 mm | 2.5 ml | 50 J/cm2, 30 ms | 100.0 | |
| | 130 W) + SS | | | | | | | |
| 120 | 16 mm Al Immersion(cont, | 28% | 1 | about 8 mm | 2.5 ml | 50 J/cm2, 30 ms | 100.0 | |
| | 130 W) + SS | | | | | | | |
| 121 | 20 mm ABS Immersion(cont, | 28% | 1 | about 8 mm | 4.0 ml | 50 J/cm2, 30 ms | 86.4 | |
| | 130 W) + SS | | | | | | | |
| 122 | 20 mm ABS Immersion(cont, | 28% | 1 | about 8 mm | 4.0 ml | 50 J/cm2, 30 ms | 61.1 | |
| | 130 W) + SS | | | | | | | |
| 123 | Average of two rows above | 28% | 2 | about 8 mm | 4.0 ml | 50 J/cm2, 30 ms | 73.7 | 17.9 |
| 124 | 16 mm Al Immersion(cont, | 28% | 1 | about 8 mm | 2.5 ml | 50 J/cm2, 30 ms | 100.0 | |
| | 130 W) + SS | | | | | | | |
| 125 | 16 mm Al Immersion(cont, | 28% | 1 | about 8 mm | 2.5 ml | 50 J/cm2, 30 ms | 100.0 | |
| | 130 W) + SS | | | | | | | |
| 126 | Average of two rows above | 28% | 2 | about 8 mm | 2.5 ml | 50 J/cm2, 30 ms | 100.0 | 0.0 |
| 127 | 16 mm Al Immersion(cont, | 28% | 1 | about 8 mm | 2.5 ml | 50 J/cm2, 30 ms | 84.2 | |
| | 130 W) + SS | | | | | | | |
| 128 | 16 mm Al Immersion(cont, | 28% | 1 | about 8 mm | 2.5 ml | 50 J/cm2, 30 ms | 81.3 | |
| | 130 W) + SS | | | | | | | |
| 129 | 16 mm Al Immersion(cont, | 28% | 1 | about 8 mm | 2.5 ml | 50 J/cm2, 30 ms | 85.7 | |
| | 130 W) + SS | | | | | | | |
| 130 | Average of three rows above | 28% | 3 | about 8 mm | 2.5 ml | 50 J/cm2, 30 ms | 83.7 | 2.3 |
| 131 | 16 mm ABS Immersion(cont, | 28% | 1 | about 8 mm | 2.5 ml | 50 J/cm2, 30 ms | 100.0 | |
| | 130 W) + SS | | | | | | | |
| 132 | 16 mm ABS Immersion(cont, | 28% | 1 | about 8 mm | 2.5 ml | 50 J/cm2, 30 ms | 100.0 | |
| | 130 W) + SS | | | | | | | |
| 133 | 16 mm ABS Immersion(cont, | 28% | 1 | about 8 mm | 2.5 ml | 50 J/cm2, 30 ms | 71.4 | |
| | 130 W) + SS | | | | | | | |
| 134 | Average of three rows above | 28% | 3 | about 8 mm | 2.5 ml | 50 J/cm2, 30 ms | 90.5 | 16.5 |
| 135 | THC Immersion(cont, 130 W), ABS, | 28% | 1 | about 8 mm | 4.5 ml | 50 J/cm2, 30 ms | 82.4 | |
| | 20 mm | | | | | | | |
| 136 | THC Immersion(cont, 130 W), ABS, | 28% | 1 | about 8 mm | 4.5 ml | 50 J/cm2, 30 ms | 70.4 | |
| | 20 mm | | | | | | | |
| 137 | THC Immersion(cont, 130 W), ABS, | 28% | 1 | about 8 mm | 3.5 ml | 50 J/cm2, 30 ms | 100.0 | |
| | 20 mm | | | | | | | |
| 138 | THC Immersion(cont, 130 W), ABS, | 28% | 1 | about 8 mm | 3.5 ml | 50 J/cm2, 30 ms | 77.8 | |
| | 20 mm | | | | | | | |
| 139 | THC Immersion(cont, 130 W), ABS, | 28% | 1 | about 8 mm | 3.5 ml | 50 J/cm2, 30 ms | 80.8 | |
| | 20 mm | | | | | | | |
| 140 | THC Immersion(cont, 130 W), ABS, | 28% | 1 | about 8 mm | 3.5 ml | 50 J/cm2, 30 ms | 88.9 | |
| | 20 mm | | | | | | | |
| 141 | Average of six rows above | 28% | 6 | about 8 mm | 4.5 ml | 50 J/cm2, 30 ms | 83.4 | 10.1 |
| 142 | FDC Immersion(cont, 130 W) | 28% | 1 | about 8 mm | 2.0 ml | 50 J/cm2, 30 ms | 100.0 | |
| 143 | FDC Immersion(cont, 130 W) | 28% | 1 | about 8 mm | 2.0 ml | 50 J/cm2, 30 ms | 64.3 | |
| 144 | FDC Immersion(cont, 130 W) | 28% | 1 | about 8 mm | 2.0 ml | 50 J/cm2, 30 ms | 90.5 | |
| 145 | FDC Immersion(cont, 130 W) | 28% | 1 | about 8 mm | 2.0 ml | 50 J/cm2, 30 ms | 100.0 | |
| 146 | Average of four rows above | 28% | 4 | about 8 mm | 2.0 ml | 50 J/cm2, 30 ms | 88.7 | 16.9 |
| 147 | FDC Immersion(cont, 130 W) + SS- | 28% | 1 | about 8 mm | 2.0 ml | 50 J/cm2, 30 ms | 91.3 | |
| | DIA | | | | | | | |
| 148 | FDC Immersion(cont, 130 W) + SS- | 28% | 1 | about 8 mm | 2.0 ml | 50 J/cm2, 30 ms | 84.0 | |
| | DIA | | | | | | | |
| 149 | Average of two rows above | 28% | 2 | about 8 mm | 2.0 ml | 50 J/cm2, 30 ms | 83.9 | 14.4 |
| 150 | FDC Immersion(cont, 130 W) + SS | 28% | 1 | about 8 mm | 2.0 ml | 50 J/cm2, 30 ms | 91.3 | |
| | OD75 | | | | | | | |
| 151 | FDC Immersion(cont, 130 W) + SS | 28% | 1 | about 8 mm | 2.0 ml | 50 J/cm2, 30 ms | 81.3 | |
| | OD75 | | | | | | | |
| 152 | FDC Immersion(cont, 130 W) + SS | 28% | 1 | about 8 mm | 2.0 ml | 50 J/cm2, 30 ms | 92.3 | |
| | OD75 | | | | | | | |
| 153 | FDC Immersion(cont, 130 W) + SS | 28% | 1 | about 8 mm | 2.0 ml | 50 J/cm2, 30 ms | 80.0 | |
| | OD75 | | | | | | | |
| 154 | Average of four rows above | 28% | 4 | about 8 mm | 2.0 ml | 50 J/cm2, 30 ms | 86.2 | 6.5 |
| 155 | FDC Immersion(cont, 130 W) + SS | 28% | 1 | about 8 mm | 2.0 ml | 50 J/cm2, 30 ms | 90.5 | |
| | OD125 | | | | | | | |
| 156 | FDC Immersion(cont, 130 W) + SS | 28% | 1 | about 8 mm | 2.0 ml | 50 J/cm2, 30 ms | 72.2 | |
| | OD125 | | | | | | | |
| 157 | FDC Immersion(cont, 130 W) + SS | 28% | 1 | about 8 mm | 2.0 ml | 50 J/cm2, 30 ms | 89.5 | |
| | OD125 | | | | | | | |
| 158 | FDC Immersion(cont, 130 W) + SS | 28% | 1 | about 8 mm | 2.0 ml | 50 J/cm2, 30 ms | 92.3 | |
| | OD125 | | | | | | | |
| 159 | Average of four rows above | 28% | 4 | about 8 mm | 2.0 ml | 50 J/cm2, 30 ms | 86.1 | 9.3 |
| 160 | THC Immersion(cont, 130 W), Al, | 28% | 1 | about 8 mm | 4.5 ml | 50 J/cm2, 30 ms | 47.4 | |
| | 16 mm, Al-plate | | | | | | | |
| 161 | THC Immersion(cont, 130 W), Al, | 28% | 1 | about 8 mm | 4.5 ml | 50 J/cm2, 30 ms | 55.6 | |
| | 16 mm, Al-plate | | | | | | | |
| 162 | THC Immersion(cont, 130 W), Al, | 28% | 1 | about 8 mm | 3.5 ml | 50 J/cm2, 30 ms | 33.3 | |
| | 16 mm, Al-plate | | | | | | | |
| 163 | Average of three rows above | 28% | 3 | about 8 mm | 4.5 ml | 50 J/cm2, 30 ms | 45.4 | 11.2 |
| 164 | THC Immersion(cont, 130 W), Al, | 28% | 1 | about 8 mm | 4.5 ml | 50 J/cm2, 30 ms | 46.2 | |
| | 16 mm, Al-plate | | | | | | | |
| 165 | THC Immersion(cont, 130 W), Al, | 28% | 1 | about 8 mm | 4.5 ml | 50 J/cm2, 30 ms | 76.9 | |
| | 16 mm, Al-plate | | | | | | | |
| 166 | Average of two rows above | 28% | 2 | about 8 mm | 4.5 ml | 50 J/cm2, 30 ms | 61.5 | 21.8 |
| 167 | FDC-top only, Imm. (cont, 130 W), | 28% | 1 | about 8 mm | 4.5 ml | 50 J/cm2, 30 ms | 93.8 | |
| | glass 16 mm, Al plate | | | | | | | |
| 168 | FDC-top only, Imm. (cont, 130 W), | 28% | 1 | about 8 mm | 4.5 ml | 50 J/cm2, 30 ms | 95.2 | |
| | glass 16 mm, Al plate | | | | | | | |
| 169 | Average of two rows above | 28% | 2 | about 8 mm | 4.5 ml | 50 J/cm2, 30 ms | 94.5 | 1.1 |
| 170 | THC Imm. (cont, 130 W), ABS, 20 mm, | 28% | 1 | about 6-7 mm | 4.5 ml | 50 J/cm2, 30 ms | 87.5 | |
| | H2O, Receiver H2O | | | | | | | |
| 171 | THC Imm. (cont, 130 W), ABS, 20 mm, | 28% | 1 | about 6-7 mm | 4.5 ml | 50 J/cm2, 30 ms | 90.9 | |
| | H2O, Receiver H2O | | | | | | | |
| 172 | Average of two rows above | 28% | 2 | about 6-7 mm | 4.5 ml | 50 J/cm2, 30 ms | 89.2 | 2.4 |
| 173 | THC Imm. (cont, 130 W), Al, 16 mm, | 28% | 1 | about 8 mm | 4.5 ml | 50 J/cm2, 30 ms | 87.5 | |
| | H2O, Receiver | | | | | | | |
| 174 | THC Imm. (cont, 130 W), Al, 16 mm, | 28% | 1 | about 8 mm | 4.5 ml | 50 J/cm2, 30 ms | 93.8 | |
| | H2O, Receiver | | | | | | | |
| 175 | Average of two rows above | 28% | 2 | about 8 mm | 4.5 ml | 50 J/cm2, 30 ms | 90.6 | 4.4 |
| 176 | THC Imm. (cont, 130 W), ABS, 20 mm, | 28% | 1 | about 8 mm | 4.5 ml | 50 J/cm2, 30 ms | 82.4 | |
| | Air Receiver | | | | | | | |
| 177 | THC Imm. (cont, 130 W), ABS, 20 mm, | 28% | 1 | about 8 mm | 4.5 ml | 50 J/cm2, 30 ms | 87.5 | |
| | Air Receiver | | | | | | | |
| 178 | THC Imm. (cont, 130 W), ABS, 20 mm, | 28% | 1 | about 8 mm | 4.5 ml | 50 J/cm2, 30 ms | 68.2 | |
| | Air Receiver | | | | | | | |
| 179 | THC Imm. (cont, 130 W), ABS, 20 mm, | 28% | 1 | about 8 mm | 4.5 ml | 50 J/cm2, 30 ms | 100.0 | |
| | Air Receiver | | | | | | | |
| 180 | Average of four rows above | 28% | 4 | about 8 mm | 4.5 ml | 50 J/cm2, 30 ms | 84.5 | 13.2 |
| 181 | THC Imm. (cont, 130 W), ABS, 20 mm, | 28% | 1 | about 8 mm | 4.5 ml | 50 J/cm2, 30 ms | 94.4 | |
| | H2O, Receiver, beef T-bone, | | | | | | | |
| | with cartilige | | | | | | | |
| 182 | THC Imm. (cont, 130 W), ABS, 20 mm, | 28% | 1 | about 8 mm | 4.5 ml | 50 J/cm2, 30 ms | 93.8 | |
| | H2O, Receiver, beef T-bone, | | | | | | | |
| | with cartilige | | | | | | | |
| 183 | Average of two rows above | 28% | 2 | about 8 mm | 4.5 ml | 50 J/cm2, 30 ms | 94.1 | 0.5 |
| 184 | THC Imm. (cont, 130 W), ABS, 20 mm, | 28% | 1 | about 8 mm | 4.5 ml | 50 J/cm2, 30 ms | 86.4 | |
| | H2O, Receiver, beef T-bone, | | | | | | | |
| | w/o cartilige | | | | | | | |
| 185 | THC Imm. (cont, 130 W), ABS, 20 mm, | 28% | 1 | about 8 mm | 4.5 ml | 50 J/cm2, 30 ms | 80.0 | |
| | H2O, Receiver, beef T-bone, | | | | | | | |
| | w/o cartilige | | | | | | | |
| 186 | Average of two rows above | 28% | 2 | about 8 mm | 4.5 ml | 50 J/cm2, 30 ms | 83.2 | 4.5 |
| 187 | THC Imm. (cont, 130 W), ABS, 20 mm, | 28% | 1 | about 8 mm | 4.5 ml | 50 J/cm2, 30 ms | 100.0 | |
| | H2O, Receiver, pig bone, w/o | | | | | | | |
| | cartilige | | | | | | | |
| 188 | THC Imm. (cont, 130 W), ABS, 20 mm, | 28% | 1 | about 8 mm | 4.5 ml | 50 J/cm2, 30 ms | 100.0 | |
| | H2O, Receiver, pig bone, w/o | | | | | | | |
| | cartilige | | | | | | | |
| 189 | Average of two rows above | 28% | 2 | about 8 mm | 4.5 ml | 50 J/cm2, 30 ms | 100.0 | 0.0 |
| 190 | THC Imm. (cont, 130 W), FDC, 16 mm, | 28% | 1 | about 8 mm | 4.5 ml | 50 J/cm2, 30 ms | 87.5 | |
| | H2O, Receiver, pig bone, w/o | | | | | | | |
| | cartilige | | | | | | | |
| 191 | THC Imm. (cont, 130 W), FDC, 16 mm, | 28% | 1 | about 8 mm | 4.5 ml | 50 J/cm2, 30 ms | 92.3 | |
| | H2O, Receiver, pig bone, w/o | | | | | | | |
| | cartilige | | | | | | | |
| 192 | Average of two rows above | 28% | 2 | about 8 mm | 4.5 ml | 50 J/cm2, 30 ms | 89.9 | 3.4 |
| 193 | FDC Imm. (cont, 130 W), 75OD | 28% | 1 | about 8 mm | 4.5 ml | 50 J/cm2, 30 ms | 85.7 | |
| 194 | FDC Imm. (cont, 130 W), 75OD | 28% | 1 | about 8 mm | 4.5 ml | 50 J/cm2, 30 ms | 70.8 | |
| 195 | Average of two rows above | 28% | 2 | about 8 mm | 4.5 ml | 50 J/cm2, 30 ms | 78.3 | 10.5 |
| 196 | FDC Imm. (cont, 130 W), 20 mm | 28% | 1 | about 8 mm | 4.5 ml | 50 J/cm2, 30 ms | 100.0 | |
| | US F78 | | | | | | | |
| 197 | FDC Imm. (cont, 130 W), 20 mm | 28% | 1 | about 8 mm | 4.5 ml | 50 J/cm2, 30 ms | 100.0 | |
| | US F78 | | | | | | | |
| 198 | Average of two rows above | 28% | 2 | about 8 mm | 4.5 ml | 50 J/cm2, 30 ms | 100.0 | 0.0 |
| 199 | FDC Imm. (cont, 130 W), 20 mm | 28% | 1 | about 8 mm | 4.5 ml | 50 J/cm2, 30 ms | 89.2 | |
| | horn | | | | | | | |
| 200 | FDC Imm. (cont, 130 W), 20 mm | 28% | 1 | about 8 mm | 4.5 ml | 50 J/cm2, 30 ms | 68.5 | |
| | horn | | | | | | | |
| 201 | Average of two rows above | 28% | 2 | about 8 mm | 4.5 ml | 50 J/cm2, 30 ms | 78.9 | 14.6 |
| 202 | FDC Imm. (cont, 130 W), 20 mm | 28% | 1 | about 8 mm | 4.5 ml | 50 J/cm2, 30 ms | 100.0 | |
| | horn | | | | | | | |
| 203 | FDC Imm. (cont, 130 W), 20 mm | 28% | 1 | about 8 mm | 4.5 ml | 50 J/cm2, 30 ms | 69.0 | |
| | horn | | | | | | | |
| 204 | Average of two rows above | 28% | 2 | about 8 mm | 4.5 ml | 50 J/cm2, 30 ms | 84.5 | 21.9 |
| 205 | FDC Imm. (cont, 130 W), 13 mm | 20% | 1 | 12 mm | 4.5 ml | 50 J/cm2, 30 ms | 92.9 | |
| | horn | | | | | | | |
| 206 | FDC Imm. (cont, 130 W), 13 mm | 20% | 1 | 12 mm | 4.5 ml | 50 J/cm2, 30 ms | 78.6 | |
| | horn | | | | | | | |
| 207 | Average of two rows above | 20% | 2 | 12 mm | 4.5 ml | 50 J/cm2, 30 ms | 85.7 | 10.1 |
| 208 | FDC Imm. (cont, 130 W), 13 mm | 20% | 1 | 15 mm | 4.5 ml | 50 J/cm2, 30 ms | 81.8 | |
| | horn | | | | | | | |
| 209 | FDC Imm. (cont, 130 W), 13 mm | 20% | 1 | 15 mm | 4.5 ml | 50 J/cm2, 30 ms | 87.5 | |
| | horn | | | | | | | |
| 210 | Average of two rows above | 20% | 2 | 15 mm | 4.5 ml | 50 J/cm2, 30 ms | 84.7 | 4.0 |

| | | M | N | O | P | | | |
|---|---|---|---|---|---|---|---|---|
| | A | SG | Std. | Deep | Std. | Q | | R |
| 1 | Description | rate | Dev | SG | Dev. | MaxTemp | | Additional Description/comments |
| 2 | Massage | | | | | | | |
| 3 | FDC Immersion | | | | | | | |
| 4 | FDC Immersion | | | | | | | |
| 5 | FDC Immersion | 16.7 | | | | 38 | C. | |
| 6 | FDC Immersion | 20 | | | | 43 | C. | |
| 7 | FDC Immersion | | | | | 38 | C. | A little SGs |
| 8 | FDC Immersion | | | | | | | |
| 9 | FDC Immersion | | | | | | | A little SGs |
| 10 | FDC Immersion | | | | | | | |
| 11 | Direct contact | | | | | | | |
| 12 | FDC Immersion(cont, 130 W) | 38.1 | | 9.5 | | 40.8 | C. | |
| 13 | FDC Immersion(cont, 130 W) | 20.0 | | 5.0 | | 41.9 | C. | Little SGs |
| 14 | FDC Immersion(cont, 130 W) | 47.6 | | 4.8 | | 40.8 | C. | |
| 15 | Average of three rows above | 35.2 | 14.0 | 6.4 | 2.7 | 40.8 | C. | |
| 16 | FDC Immersion(cont) | 25.0 | | 21.5 | | 39.8 | C. | |
| 17 | FDC Immersion(cont) | 18.8 | | 12.6 | | 41.5 | C. | |
| 18 | Average of two rows above | 21.9 | 4.4 | 17.0 | 6.3 | 39.8 | C. | |
| 19 | FDC Immersion(cont) | 7.7 | | | | 40.8 | C. | A little SGs |
| 20 | FDC Immersion(cont) | 35 | | 0.0 | | 41.5 | C. | |
| 21 | Average of two rows above | 21.4 | 19.3 | 0.0 | | 41.5 | C. | |
| 22 | FDC Immersion(cont, 130 W) + Ace | 41 | | 17.6 | | 41.3 | C. | Acetone + US 1 min(100% duty) |
| 23 | FDC Immersion + Acetone | 15.8 | | 0.0 | | 38 | C. | Acetone 2 min |
| 24 | FDC Immersion + Acetone | 28 | | 28.0 | | 43 | C. | Acetone + US 2 min |
| 25 | FDC Immersion + Acetone | 36.4 | | 32.0 | | | | Acetone + US 4 min |
| 26 | FDC Immersion + Acetone + Vacuum | 39.1 | | 26.2 | | | | Acetone + US 2 min + Vac dry 1 min |
| 27 | FDC Immersion(cont) + Acetone | 52 | | 0.0 | | 41.5 | C. | Acetone + US 1 min(100% duty) |
| 28 | FDC Immersion(Ace, | 45 | | 5.0 | | | | Acetone + US 1 min(100% duty) |
| 29 | FDC Immersion(cont) + Ace | 50.0 | | 30.0 | | 38.8 | C. | Acetone + US 1 min(100% duty) |
| 30 | FDC Immersion(cont) + Ace | 55.0 | | 30.3 | | 38 | C. | Acetone + US 1 min(100% duty) |
| 31 | FDC Immersion(cont) + Ace | 41.0 | | 0.0 | | 38 | C. | Acetone + US 1 min(100% duty) |
| 32 | FDC Immersion(cont) + Ace | 31.0 | | 23.3 | | 38 | C. | Acetone + US 50 sec(100% duty) |
| 33 | Average of four rows above | 44.3 | 10.6 | 20.9 | 14.3 | 38.8 | C. | Acetone + US 1 min(100% duty) |
| 34 | FDC Immersion(cont) + Isopropanol | 41.0 | | 23.4 | | 39 | C. | isopropanol + US 1 min(100% duty) |
| 35 | FDC Immersion(cont) + Isopropanol | 41.0 | | 13.5 | | 38 | C. | Isopropanol + US 1 min(100% duty) |
| 36 | FDC Immersion(cont) + Isopropanol | 52.0 | | 14.0 | | 38 | C. | Isopropanol + US 1 min(100% duty) |
| 37 | FDC Immersion(cont) + Isopropanol | 31.0 | | 20.8 | | 38 | C. | Isopropanol + US 50 sec(100% duty) |
| 38 | Average of four rows above | 41.3 | 8.6 | 17.9 | 4.9 | 39 | C. | Isopropanol + US 1 min(100% duty) |
| 39 | FDC Immersion(cont) + Sebashell | 42.0 | | 26.5 | | 37 | C. | Sebashell + US 1 min(100% duty) |
| 40 | FDC Immersion(cont) + Sebashell | 42.0 | | 16.8 | | 37 | C. | Sebashell + US 1 min(100% duty) |
| 41 | FDC Immersion(cont) + Sebashell | 65.0 | | 50.1 | | 37 | C. | Sebashell + US 1 min(100% duty) |
| 42 | Average of three rows above | 49.7 | 13.3 | 31.1 | 17.1 | 37 | C. | Sebashell + US 1 min(100% duty) |
| 43 | FDC Immersion(cont) + Water | 42.0 | | 16.8 | | 37 | C. | Water + US 1 min(100% duty) |
| 44 | FDC Immersion(cont) + Water | 52.0 | | 15.1 | | 37 | C. | Water + US 1 min(100% duty) |
| 45 | FDC Immersion(cont) + Water | 20.0 | | 10.0 | | 37 | C. | Water + US 1 min(100% duty) |
| 45 | Average of three rows above | 38.0 | 16.4 | 14.0 | 3.5 | 37 | C. | Water + US 1 min(100% duty) |
| 47 | FDC Immersion(cont) + Ethanol | 38.0 | | 25.5 | | 39 | C. | Ethanol + US 1 min(100% duty) |
| 48 | FDC Immersion(cont) + Ethanol | 22.0 | | 5.5 | | 38 | C. | Ethanol + US 50 sec(100% duty) |
| 49 | FDC Immersion(cont) + Ethanol | 10.0 | | 10.0 | | 38 | C. | Ethanal + US 50 sec(100% duty) |
| 50 | Average of three rows above | 23.3 | 14.0 | 13.7 | 10.5 | 39 | C. | Ethanol + US 1 min(100% duty) |
| 51 | FDC Immersion(cont) + DMSO | 45.0 | | 0.0 | | 39 | C. | DMSO + US 50 sec(100% duty) (45 C.) |
| 52 | FDC Immersion(cont) + DMSO | 33.0 | | 7.3 | | 39 | C. | DMSO + US 50 sec(100% duty) (42 C.) |
| 53 | Average of two rows above | 39.0 | 8.5 | 3.6 | 5.1 | 39 | C. | DMSO + US 50 sec(100% duty) (45 C.) |
| 54 | FDC Immersion(cont) + SS(twice) | 33.0 | | 33.0 | | 37 | C. | Sebashell + US 1 min(100% duty, twice) |
| 55 | FDC Immersion(cont) + SS(twice) | 79.0 | | 50.6 | | 37 | C. | Sebashell + US 1 min(100% duty, twice) |
| 56 | FDC Immersion(cont) + SS(twice) | 84.0 | | 33.6 | | 37 | C. | Sebashell + US 1 min(100% duty, twice) |
| 57 | Average of three rows above | 65.3 | 28.1 | 39.1 | 10.0 | 37 | C. | Sebashell + US 1 min(100% duty, twice) |
| 58 | FDC Immersion(cont) + SS(twice, | 59 | | 31.9 | | 37 | C. | Sebashell + US 1 min(100% duty, twice) |
| | recycle) | | | | | | | |
| 59 | FDC Immersion(cont) + SS(twice, | 74 | | 39.2 | | 37 | C. | Sebashell + US 1 min(100% duty, twice) |
| | recycle) | | | | | | | |
| 60 | Average of two rows above | 66.5 | 10.6 | 35.5 | 5.2 | 37 | C. | Sebashell + US 1 min(100% duty, twice) |
| 61 | FDC Immersion (cont, 130 W) + | 63.2 | | 26.3 | | 40.5 | | Sebashell + US 45 sec(100% duty) |
| | Sebashell | | | | | | | |
| 62 | FDC Immersion(cont, | 75.0 | | 12.5 | | 40.5 | | Sebashell + US 45 sec(100% duty) |
| | 130 W) + Sebashell | | | | | | | |
| 63 | FDC Immersion(cont, | 55.0 | | 15.0 | | 40.5 | | Sebashell + US 45 sec(100% duty) |
| | 130 W) + Sebashell | | | | | | | |
| 64 | FDC Immersion(cont, | 64.4 | 10.1 | 17.9 | 7.4 | 40.5 | | Sebashell + US 45 sec(100% duty) |
| | 130 W) + Sebashell | | | | | | | |
| 65 | FDC Immersion(cont, | 70.0 | | 10.0 | | 40.5 | | Acetone + US 1 min(100% duty) |
| | 130 W) + Acetone | | | | | | | |
| 66 | FDC Immersion(cont, 130 W) + SS in | 68.0 | | 25.8 | | 26.8-40.2 | | Sebashell + US 30 sec(100% duty) 28.3-39.8 C. |
| | 33 C. | | | | | | | |
| 67 | FDC Immersion(cont, 130 W) + SS in | 54.0 | | 23.2 | | 26.5-40.8 | | Sebashell + US 45 sec(100% duty) 26.4-40.4 C. |
| | 33 C. | | | | | | | |
| 68 | FDC Immersion(cont, 130 W) + SS in | 35.0 | | 15.1 | | 29.7-42.6 | | Sebashell + US 27 sec(100% duty) 29.4-41 C. |
| | 33 C. | | | | | | | |
| 69 | FDC Immersion(cont, 130 W) + SS in | 40.0 | | 15.2 | | 28-40.9 | | Sebashell + US 28 sec(100% duty) 28.8-40.4 C. |
| | 33 C. | | | | | | | |
| 70 | FDC Immersion(cont, 130 W, | 4/? | | | | 27-40.5 | | HumanSkin Temp: 30.5-36 |
| | human) in 33 C. | | | | | | | |
| 71 | FDC Immersion(cont, 130 W, | 0.0 | | | | 29-42 | | Human Skin Temp: 29.4-35.2 |
| | human w/hair) in 33 C. | | | | | | | |
| 72 | FDC Immersion(cont, 600 W, | 2 | | | | 21-40.7 | | |
| | human) | (possible) | | | | | | |
| 73 | Massage(rabbit ear) | | | | | | | |
| 74 | FDC Immersion(cont, 130 W, | | | | | 21-40.7 | | Sebashell + US 30 sec(100% duty) 29.4-41 C. |
| | rabbit) | | | | | | | |
| 75 | FDC Immersion(cont, 130 W) + SS | 50.0 | | 16.5 | | 29.7-42.6 | | Sebashell + US 30 sec(100% duty) 29.4-41 C. |
| 76 | FDC Immersion(cont, 130 W) + SS | 61.0 | | 27.5 | | 28-40.9 | | Sebashell + US 20 sec(100% duty) 28.8-40.4 C. |
| 77 | Sonoprep Immersion(cont) + SS | 15.0 | | 0.0 | | 28-40.9 | | Sebashell + US 50 sec(100% duty) 28.8-40.4 C. |
| 78 | Sonoprep Immersion(cont) + SS | 60.0 | | 6.6 | | 28-40.9 | | Sebashell + US 35 sec(100% duty) 28.8-40.4 C. |
| 79 | Sonoprep Immersion(cont) + SS | 9.0 | | 9.0 | | 28-40.9 | | Sebashell + US 16 sec(100% duty) 28.8-40.4 C. |
| 80 | Sonoprep Immersion(cont) + SS | 33.0 | | 0.0 | | 28-40.9 | | Sebashell + US 83 sec(100% duty) 28.8-40.4 C. |
| 81 | FDC Immersion(cont, 130 W) + SS | 25.0 | | 0.0 | | | | |
| 82 | FDC Immersion(cont, 130 W) + SS | 10.0 | | 0.0 | | | | |
| 83 | FDC Immersion(cont, 130 W) + SS | 40.0 | | 15.0 | | | | |
| 84 | FDC Immersion(cont, 130 W) + SS | 64.3 | | 50.0 | | | | |
| 85 | FDC Immersion(cont, 130 W) + SS | 60.0 | | 19.8 | | | | |
| 86 | Average of three rows above | 54.8 | 13.0 | 28.3 | 19.0 | | | Sebashell + US 30 sec(100% duty) 29.4-41 C. |
| 87 | FDC Immersion(cont, 130 W) + SS | 42.1 | | 21.1 | | | | |
| 88 | FDC Immersion(cont, 130 W) + SS | 21.4 | | 14.3 | | | | |
| 89 | FDC Immersion(cont, 130 W) + SS | 50.0 | | 50.0 | | | | |
| 90 | Average of three rows above | 37.8 | 14.8 | 28.4 | 19.0 | | | Sebashell + US 30 sec(100% duty) 29.4-41 C. |
| 91 | FDC Immersion(cont, 130 W) + SS | 37.5 | | 25.0 | | | | |
| 92 | FDC Immersion(cont, 130 W) + SS | 54.5 | | 18.2 | | | | |
| 93 | Average of two rows above | 46.0 | 12.1 | 21.6 | 4.8 | | | Sebashell + US 30 sec(100% duty) 29.4-41 C. |
| 94 | FDC Immersion(cont, 130 W) + SS | 37.5 | | 12.5 | | | | |
| 95 | FDC Immersion(cont, 130 W) + SS | 28.6 | | 14.3 | | | | |
| 96 | Average of two rows above | 33.0 | 6.3 | 13.4 | 1.3 | | | Sebashell + US 30 sec(100% duty) 29.4-41 C. |
| 97 | FDC Immersion(cont, 130 W) + SS | 36.4 | | 18.2 | | | | Sebashell + US 30 sec(100% duty) 29.4-41 C. |
| 98 | FDC Immersion(cont, 130 W) + SS | 60.0 | | 30.0 | | | | Sebashell + US 30 sec(100% duty) 29.4-41 C. |
| 99 | Average of two rows above | 48.2 | 16.7 | 24.1 | 8.4 | | | Sebashell + US 30 sec(100% duty) 29.4-41 C. |
| 100 | FDC Immersion(cont, 130 W) + SS | 42.1 | | 31.6 | | | | Sebashell + US 30 sec(100% duty) 29.4-41 C. |
| 101 | FDC Immersion(cont, 130 W) + SS | 70.0 | | 40.0 | | | | Sebashell + US 30 sec(100% duty) 29.4-41 C. |
| 102 | Average of two rows above | 56.1 | 19.7 | 35.8 | 6.0 | | | Sebashell + US 30 sec(100% duty) 29.4-41 C. |
| 103 | FDC Immersion(cont, 130 W) + SS | 16.7 | | 8.3 | | | | Sebashell + US 30 sec(100% duty) 29.4-41 C. |
| 104 | FDC Immersion(cont, 130 W) + SS | 57.9 | | 36.8 | | | | Sebashell + US 30 sec(100% duty) 29.4-41 C. |
| 105 | Average of two rows above | 37.3 | 29.2 | 22.6 | 20.2 | | | Sebashell + US 30 sec(100% duty) 29.4-41 C. |
| 106 | FDC Immersion(cont, 130 W) + SS | 28.6 | | 7.1 | | | | Sebashell + US 30 sec(100% duty) 29.4-41 C. |
| 107 | FDC Immersion(cont, 130 W) + SS | 40.0 | | 40.0 | | | | Sebashell + US 30 sec(100% duty) 29.4-41 C. |
| 108 | Average of two rows above | 34.3 | 8.1 | 23.6 | 23.2 | | | Sebashell + US 30 sec(100% duty) 29.4-41 C. |
| 109 | Average of rows 83-85, 100-101, | 40.5 | 17.0 | 25.5 | 14.4 | | | Sebashell + US 30 sec(100% duty) 29.4-41 C. |
| | and 106-107 above | | | | | | | |
| 110 | Average of rows 87-89, 97-98, | 49.3 | 15.4 | 29.1 | 15.5 | | | Sebashell + US 30 sec(100% duty) 29.4-41 C. |
| | and 103-104 above | | | | | | | |
| 111 | FDC Immersion(cont, 130 W) + SS | | 4.8 | | 0.0 | 4.8 | | Sebashell + US 30 sec(100% duty) 29.4-41 C. |
| 112 | FDC Immersion(cont, 130 W) + SS | 5.0 | | 0.0 | | | | Sebashell + US 30 sec(100% duty) 29.4-41 C. |
| 113 | Average of two rows above | 4.9 | 0.2 | 2.4 | 3.4 | | | Sebashell + US 30 sec(100% duty) 29.4-41 C. |
| 114 | THC Immersion(cont, 130 W), ABS, | 22.2 | | 0.0 | | | | |
| | 20 mm | | | | | | | |
| 115 | THC Immersion(cont, 130 W) + SS, | 33.3 | | 10.0 | | | | Sebashell + US 30 sec(100% duty) 20-25 C. |
| | ABS, 20 mm | | | | | | | |
| 116 | 20 mm ABS Immersion(cont, | 11.0 | | 6.0 | | 27-38 | | Sebashell + US 30 sec(100% duty) 24-36 C. |
| | 130 W) + SS | | | | | | | |
| 117 | 18 mm ABS Immersion(cont, | No SGs | | | | 27-41 | | Sebashell + US 30 sec(100% duty) 21-35 C. |
| | 130 W) + SS | | | | | | | |
| 118 | 18 mm Al Immersion(cont, | No SGs | | | | 27-37 | | Sebashell + US 30 sec(100% duty) 21-33 C. |
| | 130 W) + SS | | | | | | | |
| 119 | 16 mm ABS Immersion(cont, | 6.0 | | | | 25-40 | | Sebashell + US 30 sec(100% duty) 23-39 C. |
| | 130 W) + SS | | | | | | | |
| 120 | 16 mm Al Immersion(cont, | 22.0 | | 22.0 | | 27-37 | | Sebashell + US 30 sec(100% duty) 21-33 C. |
| | 130 W) + SS | | | | | | | |
| 121 | 20 mm ABS Immersion(cont, | 36.4 | | 31.8 | | 28-40 | | Sebashell + US 30 sec(100% duty) 22-36 C. |
| | 130 W) + SS | | | | | | | |
| 122 | 20 mm ABS Immersion(cont, | 37.5 | | 31.3 | | 29-40 | | Sebashell + US 30 sec(100% duty) 26-38 C. |
| | 130 W) + SS | | | | | | | |
| 123 | Average of two rows above | 36.9 | 0.8 | 31.5 | 0.4 | 28-40 | | Sebashell + US 30 sec(100% duty) 22-36 C. |
| 124 | 16 mm Al Immersion(cont, | 30.8 | | 15.4 | | 28-39 | | Sebashell + US 30 sec(100% duty) 24-36 C. |
| | 130 W) + SS | | | | | | | |
| 125 | 16 mm Al Immersion(cont, | 16.7 | | 0.0 | | 28-38 | | Sebashell + US 30 sec(100% duty) 25-36 C. |
| | 130 W) + SS | | | | | | | |
| 126 | Average of two rows above | 23.7 | 10.0 | 7.7 | 10.9 | 28-39 | | Sebashell + US 30 sec(100% duty) 24-36 C. |
| 127 | 16 mm Al Immersion(cont, | 16.7 | | 5.6 | | 28-38 | | Sebashell + US 30 sec(100% duty) 22-33 C. |
| | 130 W) + SS | | | | | | | |
| 128 | 16 mm Al Immersion(cont, | 56.3 | | 6.3 | | 27-38 | | Sebashell + US 30 sec(100% duty) 21-34 C. |
| | 130 W) + SS | | | | | | | |
| 129 | 16 mm Al Immersion(cont, | 35.7 | | 0.0 | | 28-38 | | Sebashell + US 30 sec(100% duty) 21-33 C. |
| | 130 W) + SS | | | | | | | |
| 130 | Average of three rows above | 36.2 | 19.8 | 3.9 | 3.4 | | | |
| 131 | 16 mm ABS Immersion(cont, | 33.3 | | 11.1 | | 28-42 | | Sebashell + US 30 sec(100% duty) 22-38 C. |
| | 130 W) + SS | | | | | | | |
| 132 | 16 mm ABS Immersion(cont, | 50.0 | | 8.3 | | 27-40 | | Sebashell + US 30 sec(100% duty) 22-36 C. |
| | 130 W) + SS | | | | | | | |
| 133 | 16 mm ABS Immersion(cont, | 40.0 | | 10.0 | | 28-41 | | Sebashell + US 30 sec(100% duty) 22-36 C. |
| | 130 W) + SS | | | | | | | |
| 134 | Average of three rows above | 41.1 | 8.4 | 9.8 | 1.4 | | | |
| 135 | THC Immersion(cont, 130 W), ABS, | 26.7 | | 6.7 | | 19-38 | | |
| | 20 mm | | | | | | | |
| 136 | THC Immersion(cont, 130 W), ABS, | 33.3 | | 4.8 | | 20-41 | | |
| | 20 mm | | | | | | | |
| 137 | THC Immersion(cont, 130 W), ABS, | 47.1 | | 5.9 | | 20-37 | | |
| | 20 mm | | | | | | | |
| 138 | THC Immersion(cont, 130 W), ABS, | 47.6 | | 23.8 | | 24-36 | | |
| | 20 mm | | | | | | | |
| 139 | THC Immersion(cont, 130 W), ABS, | 38.5 | | 0.0 | | 20-38 | | |
| | 20 mm | | | | | | | |
| 140 | THC Immersion(cont, 130 W), ABS, | 33.3 | | 0.0 | | 25-42 | | |
| | 20 mm | | | | | | | |
| 141 | Average of six rows above | 37.7 | 8.3 | 6.9 | 8.8 | | | |
| 142 | FDC Immersion(cont, 130 W) | 41.7 | | 16.7 | | 22-33 | | |
| 143 | FDC Immersion(cont, 130 W) | 28.6 | | 0.0 | | 26-39 | | |
| 144 | FDC Immersion(cont, 130 W) | 66.7 | | 13.3 | | 19-39 | | |
| 145 | FDC Immersion(cont, 130 W) | 54.5 | | 18.2 | | 19-36 | | |
| 146 | Average of four rows above | 47.9 | 16.4 | 12.0 | 8.3 | | | |
| 147 | FDC Immersion(cont, 130 W) + SS- | 44.4 | | 0.0 | | 26-40 | | Sebashell + US 30 sec(100% duty) 22-35 C. |
| | DIA | | | | | | | |
| 148 | FDC Immersion(cont, 130 W) + SS- | 31.8 | | 0.0 | | 25-39 | | Sebashell + US 30 sec(100% duty) 23-38 C. |
| | DIA | | | | | | | |
| 149 | Average of two rows above | 44.9 | 19.2 | 21.1 | 17.5 | | | Sebashell + US 30 sec(100% duty) 22-35 C. |
| 150 | FDC Immersion(cont, 130 W) + SS | 53.8 | | 38.5 | | 26-40 | | Sebashell + US 30 sec(100% duty) 22-42 C. |
| | OD75 | | | | | | | |
| 151 | FDC Immersion(cont, 130 W) + SS | 21.4 | | 0.0 | | 29-41 | | Sebashell + US 30 sec(100% duty) 23-41 C. |
| | OD75 | | | | | | | |
| 152 | FDC Immersion(cont, 130 W) + SS | 33.3 | | 0.0 | | 23-40 | | Sebashell + US 30 sec(100% duty) 22-39 C. |
| | OD75 | | | | | | | |
| 153 | FDC Immersion(cont, 130 W) + SS | 0.0 | | 0.0 | | 25-41 | | Sebashell + US 30 sec(100% duty) 22-41 C. |
| | OD75 | | | | | | | |
| 154 | Average of four rows above | 27.2 | 22.5 | 9.6 | 19.2 | | | Sebashell + US 30 sec(100% duty) 22-35 C. |
| 155 | FDC Immersion(cont, 130 W) + SS | 42.9 | | 23.8 | | 26-40 | | Sebashell + US 30 sec(100% duty) 22-42 C. |
| | OD125 | | | | | | | |
| 156 | FDC Immersion(cont, 130 W) + SS | 43.8 | | 31.3 | | 29-41 | | Sebashell + US 30 sec(100% duty) 23-41 C. |
| | OD125 | | | | | | | |
| 157 | FDC Immersion(cont, 130 W) + SS | 57.9 | | 47.4 | | 23-40 | | Sebashell + US 30 sec(100% duty) 22-39 C. |
| | OD125 | | | | | | | |
| 158 | FDC Immersion(cont, 130 W) + SS | 20.0 | | 20.0 | | 25-41 | | Sebashell + US 30 sec(100% duty) 22-41 C. |
| | OD125 | | | | | | | |
| 159 | Average of four rows above | 41.1 | 15.7 | 30-6 | 12.1 | | | Sebashell + US 30 sec(100% duty) 22-35 C. |
| 160 | THC Immersion(cont, 130 W), Al, | 15.8 | | 0.0 | | 26-35 | | |
| | 16 mm, Al-plate | | | | | | | |
| 161 | THC Immersion(cont, 130 W), Al, | 5.6 | | 0.0 | | 24-36 | | |
| | 16 mm, Al-plate | | | | | | | |
| 162 | THC Immersion(cont, 130 W), Al, | 0.0 | | 0.0 | | 24-35 | | |
| | 16 mm, Al-plate | | | | | | | |
| 163 | Average of three rows above | 7.1 | 8.0 | 0.0 | 0.0 | | | |
| 164 | THC Immersion(cont, 130 W), Al, | 0.0 | | 0.0 | | 22-32 | | |
| | 16 mm, Al-plate | | | | | | | |
| 165 | THC Immersion(cont, 130 W), Al, | 23.1 | | 0.0 | | 22-31 | | |
| | 16 mm, Al-plate | | | | | | | |
| 166 | Average of two rows above | 11.5 | 16.3 | 0.0 | 0.0 | | | |
| 167 | FDC-top only, Imm. (cont, 130 W), | 31.3 | | 0.0 | | 26-40 | | Sebashell + US 30 sec(100% duty) 22-42 C. |
| | glass 16 mm, Al plate | | | | | | | |
| 168 | FDC-top only, Imm. (cont, 130 W), | 61.9 | | 4.8 | | 29-41 | | Sebashell + US 30 sec(100% duty) 23-41 C. |
| | glass 16 mm, Al plate | | | | | | | |
| 169 | Average of two rows above | 46.6 | 21.7 | 2.4 | 3.4 | | | |
| 170 | THC Imm. (cont, 130 W), ABS, 20 mm, | 62.5 | | 50.0 | | 22-37 | | Sebashell + US 30 sec(100% duty) 22-40 C. |
| | H2O, Receiver H2O | | | | | | | |
| 171 | THC Imm. (cont, 130 W), ABS, 20 mm, | 66.7 | | 41.7 | | 22-38 | | Sebashell + US 30 sec(100% duty) 23-40 C. |
| | H2O, Receiver H2O | | | | | | | |
| 172 | Average of two rows above | 64.6 | 2.9 | 45.8 | 5.9 | | | |
| 173 | THC Imm. (cont, 130 W), Al, 16 mm, | 50.0 | | 18.8 | | 22-32 | | |
| | H2O, Receiver | | | | | | | |
| 174 | THC Imm. (cont, 130 W), Al, 16 mm, | 62.5 | | 18.8 | | 22-31 | | |
| | H2O, Receiver | | | | | | | |
| 175 | Average of two rows above | 56.3 | 8.8 | 18.8 | 0.0 | | | |
| 176 | THC Imm. (cont, 130 W), ABS, 20 mm, | 53.3 | | 40.0 | | 30-38 | | Sebashell + US 30 sec(100% duty) 22-40 C. |
| | Air Receiver | | | | | | | |
| 177 | THC Imm. (cont, 130 W), ABS, 20 mm, | 60.9 | | 34.8 | | 30-38 | | Sebashell + US 30 sec(100% duty) 22-40 C. |
| | Air Receiver | | | | | | | |
| 178 | THC Imm. (cont, 130 W), ABS, 20 mm, | 36.4 | | 4.5 | | 30-38 | | Sebashell + US 30 sec(100% duty) 22-40 C. |
| | Air Receiver | | | | | | | |
| 179 | THC Imm. (cont, 130 W), ABS, 20 mm, | 57.9 | | 26.3 | | 31-41 | | Sebashell + US 30 sec(100% duty) 23-40 C. |
| | Air Receiver | | | | | | | |
| 180 | Average of four rows above | 52.1 | 10.9 | 26.4 | 15.6 | | | |
| 181 | THC Imm. (cont, 130 W), ABS, 20 mm, | 61.1 | | 27.8 | | 30-38 | | Sebashell + US 30 sec(100% duty) 22-40 C. |
| | H2O, Receiver, beef T-bone, | | | | | | | |
| | with cartilige | | | | | | | |
| 182 | THC Imm. (cont, 130 W), ABS, 20 mm, | 50.0 | | 6.3 | | 30-38 | | Sebashell + US 30 sec(100% duty) 22-40 C. |
| | H2O, Receiver, beef T-bone, | | | | | | | |
| | with cartilige | | | | | | | |
| 183 | Average of two rows above | 55.6 | 7.9 | 17.0 | 15.2 | | | |
| 184 | THC Imm. (cont, 130 W), ABS, 20 mm, | 54.5 | | 35.4 | | 30-38 | | Sebashell + US 30 sec(100% duty) 22-40 C. |
| | H2O, Receiver, beef T-bone, | | | | | | | |
| | w/o cartilige | | | | | | | |
| 185 | THC Imm. (cont, 130 W), ABS, 20 mm, | 40.0 | | 6.7 | | 30-38 | | Sebashell + US 30 sec(100% duty) 22-40 C. |
| | H2O, Receiver, beef T-bone, | | | | | | | |
| | w/o cartilige | | | | | | | |
| 186 | Average of two rows above | 47.3 | 10.3 | 21.5 | 21.0 | | | |
| 187 | THC Imm. (cont, 130 W), ABS, 20 mm, | 50.0 | | 19.2 | | 30-38 | | Sebashell + US 30 sec(100% duty) 22-40 C. |
| | H2O, Receiver, pig bone, w/o | | | | | | | |
| | cartilige | | | | | | | |
| 188 | THC Imm. (cont, 130 W), ABS, 20 mm, | 66.7 | | 27.8 | | 30-38 | | Sebashell + US 30 sec(100% duty) 22-40 C. |
| | H2O, Receiver, pig bone, w/o | | | | | | | |
| | cartilige | | | | | | | |
| 189 | Average of two rows above | 58.3 | 11.8 | 23.5 | 6.0 | | | |
| 190 | THC Imm. (cont, 130 W), FDC, 16 mm, | 56.3 | | 31.3 | | 30-38 | | Sebashell + US 30 sec(100% duty) 22-40 C. |
| | H2O, Receiver, pig bone, w/o | | | | | | | |
| | cartilige | | | | | | | |
| 191 | THC Imm. (cont, 130 W), FDC, 16 mm, | 61.5 | | 30.8 | | 30-38 | | Sebashell + US 30 sec(100% duty) 22-40 C. |
| | H2O, Receiver, pig bone, w/o | | | | | | | |
| | cartilige | | | | | | | |
| 192 | Average of two rows above | 58.9 | 3.7 | 31.0 | 0.3 | | | |
| 193 | FDC Imm. (cont, 130 W), 75OD | 25.0 | | 10.0 | | 30-38 | | Sebashell + US 30 sec(100% duty) 22-40 C. |
| 194 | FDC Imm. (cont, 130 W), 75OD | 54.2 | | 45.8 | | 30-38 | | Sebashell + US 30 sec(100% duty) 22-40 C. |
| 195 | Average of two rows above | 39.6 | 20.6 | 27.9 | 25.3 | | | |
| 196 | FDC Imm. (cont, 130 W), 20 mm | 50.0 | | 10.0 | | 30-38 | | Sebashell + US 30 sec(100% duty) 22-40 C. |
| | US F78 | | | | | | | |
| 197 | FDC Imm. (cont, 130 W), 20 mm | 81.8 | | 72.7 | | 30-38 | | Sebashell + US 30 sec(100% duty) 22-40 C. |
| | US F78 | | | | | | | |
| 198 | Average of two rows above | 65.9 | 22.5 | 41.4 | 44.4 | | | |
| 199 | FDC Imm. (cont, 130 W), 20 mm | 46.9 | | 15.6 | | | | Sebashell + US 60 sec(100% duty) 22-33 C. |
| | horn | | | | | | | |
| 200 | FDC Imm. (cont, 130 W), 20 mm | 50.0 | | 26.1 | | | | Sebashell + US 60 sec(100% duty) 22-32 C. |
| | horn | | | | | | | |
| 201 | Average of two rows above | 48.4 | 2.2 | 20.9 | 7.4 | | | |
| 202 | FDC Imm. (cont, 130 W), 20 mm | 80.0 | | 70.0 | | | | Sebashell + US 60 sec(100% duty) 22-33 C. |
| | horn | | | | | | | |
| 203 | FDC Imm. (cont, 130 W), 20 mm | 42.9 | | 25.0 | | | | Sebashell + US 60 sec(100% duty) 22-30 C. |
| | horn | | | | | | | |
| 204 | Average of two rows above | 61.4 | 26.3 | 47.5 | 31.8 | | | |
| 205 | FDC Imm. (cont, 130 W), 13 mm | 61.5 | | 23.1 | | | | Sebashell + US 60 sec(100% duty) 22-40 C. |
| | horn | | | | | | | |
| 206 | FDC Imm. (cont, 130 W), 13 mm | 54.5 | | 18.2 | | | | Sebashell + US 60 sec(100% duty) 22-40 C. |
| | horn | | | | | | | |
| 207 | Average of two rows above | 58.0 | 4.9 | 20.6 | 3.5 | | | |
| 208 | FDC Imm. (cont, 130 W), 13 mm | 27.3 | | 4.5 | | | | Sebashell + US 60 sec(100% duty) 22-40 C. |
| | horn | | | | | | | |
| 209 | FDC Imm. (cont, 130 W), 13 mm | 43.8 | | 25.0 | | | | Sebashell + US 60 sec(100% duty) 22-40 C. |
| | horn | | | | | | | |
| 210 | Average of two rows above | 35.5 | 11.7 | 14.8 | 14.5 | | | |

APPENDIX A TABLE 3

| | A | B | C | D | | E | F | G | H | |
|---|---|---|---|---|---|---|---|---|---|---|
| 1 | Description | Freq. | Transducer | Total time | | Duty cycle | Amplitude | Repeat | Distance | |
| 2 | Massage | | | 4 | min | 100% | weak | 3 | 0 | mm |
| 3 | FDC Immersion | 20 kHz | 13 mm probe | 1 | min | 50% (5 sec on/off) | 10% | 1 | 5 | mm |
| 4 | FDC Immersion | 20 kHz | 13 mm probe | 2 | min | 50% (5 sec on/off) | 10% | 4 | 5 | mm |
| 5 | FDC Immersion | 20 kHz | 13 mm probe | 3 | min | 50% (5 sec on/off) | 10% | 2 | 5 | mm |
| 6 | FDC Immersion | 20 kHz | 13 mm probe | 4 | min | 50% (5 sec on/off) | 10% | 1 | 5 | mm |
| 7 | FDC Immersion | 20 kHz | 13 mm probe | 5(3 + 2) | min | 50% (5 sec on/off) | 10% | 1 | 5 | mm |
| 8 | FDC Immersion | 20 kHz | 13 mm probe | 2 | min | 50% (5 sec on/off) | 20% | 2 | 5 | mm |
| 9 | FDC Immersion | 20 kHz | 13 mm probe | 2 | min | 50% (5 sec on/off) | 30% | 2 | 5 | mm |
| 10 | FDC Immersion | 20 kHz | 13 mm probe | 2 | min | 50% (5 sec on/off) | 10% | 4 | 2.5 | mm |
| 11 | Direct contact | 20 kHz | 13 mm probe | 2 | min | 50% (5 sec on/off) | 10% | 3 | 0 | mm |
| 12 | FDC Immersion(cont, 130 W) | 20 kHz | 13 mm probe | 50 | sec | 100% | 20% | 1 | 5 | mm |
| 13 | FDC Immersion(cont, 130 W) | 20 kHz | 13 mm probe | 50 | sec | 100% | 20% | 1 | 5 | mm |
| 14 | FDC Immersion(cont, 130 W) | 20 kHz | 13 mm probe | 40 | sec | 100% | 20% | 1 | 5 | mm |
| 15 | Average of three rows above | 30 kHz | 13 mm probe | 50 | sec | 100% | 20% | 3 | 5 | mm |
| 16 | FDC Immersion(cont) | 20 kHz | 13 mm probe | 50 | sec | 100% | 10% | 1 | 5 | mm |
| 17 | FDC Immersion (cont) | 20 kHz | 13 mm probe | 1 | min | 100% | 10% | 1 | 5 | mm |
| 18 | Average of two rows above | 20 kHz | 13 mm probe | 50 | sec | 100% | 10% | 2 | 5 | mm |
| 19 | FDC Immersion(cont) | 20 kHz | 13 mm probe | 50 | sec | 100% | 15% | 1 | 5 | mm |
| 20 | FDC Immersion(cont) | 20 kHz | 13 mm probe | 42 | sec | 100% | 15% | 1 | 5 | mm |
| 21 | Average of two rows above | 20 kHz | 13 mm probe | 42 | sec | 100% | 15% | 2 | 5 | mm |
| 22 | FDC Immersion(cont, 130 W) + Ace | 20 kHz | 13 mm probe | 50 | sec | 100% | 10% | 1 | 5 | mm |
| 23 | FDC Immersion + Acetane | 20 kHz | 13 mm probe | 2 | min | 50% (5 sec on/off) | 10% | 1 | 0 | mm |
| 24 | FDC Immersions + Acetone | 20 kHz | 13 mm probe | 2 | min | 50% (5 sec on/off) | 10% | 1 | 0 | mm |
| 25 | FDC Immersion + Acetone | 20 kHz | 13 mm probe | 2 | min | 50% (5 sec on/off) | 10% | 1 | 0 | mm |
| 26 | FDC Immersion + Acetone + | 20 kHz | 13 mm probe | 2 | min | 50% (5 sec on/off) | 10% | 1 | 0 | mm |
| | Vacuum | | | | | | | | | |
| 27 | FDC Immersion(cont) + Acetane | 20 kHz | 13 mm probe | 40 | sec | 100% | 15% | 1 | 5 | mm |
| 28 | FDC Immersion(Aca, | 20 kHz | 13 mm probe | 4 | min | 100% | 10% | 1 | 0 | mm |
| | cont) + Massage | | | | | | | | | |
| 29 | FDC Immersian(cont) + Ace | 20 kHz | 13 mm probe | 50 | sec | 100% | 10% | 1 | 5 | mm |
| 30 | FDC Immersion(cont) + Ace | 20 kHz | 13 mm probe | 50 | sec | 100% | 10% | 1 | 5 | mm |
| 31 | FDC Immersion(cont) + Ace | 20 kHz | 13 mm probe | 50 | sec | 100% | 10% | 1 | 5 | mm |
| 32 | FDC Immersion(cont) + Ace | 20 kHz | 13 mm probe | 50 | sec | 100% | 10% | 1 | 5 | mm |
| 33 | Average of four rows above | 20 kHz | 13 mm probe | 50 | sec | 100% | 10% | 4 | 5 | mm |
| 34 | FDC Immersion(cont) + Isopropanol | 20 kHz | 13 mm probe | 50 | sec | 100% | 10% | 1 | 5 | mm |
| 35 | FDC Immersion(cont) + Isopropanol | 20 kHz | 13 mm probe | 50 | sec | 100% | 10% | 1 | 5 | mm |
| 36 | FDC Immersion(cont) + Isopropanol | 20 kHz | 13 mm probe | 50 | sec | 100% | 10% | 1 | 5 | mm |
| 37 | FDC Immersion(cont) + Isopropanol | 20 kHz | 13 mm probe | 50 | sec | 100% | 10% | 1 | 5 | mm |
| 38 | Average of four rows above | 20 kHz | 13 mm probe | 50 | sec | 100% | 10% | 4 | 5 | mm |
| 39 | FDC Immersion(cont) + Sebashell | 20 kHz | 13 mm probe | 50 | sec | 100% | 10% | 1 | 5 | mm |
| 40 | FDC Immersion(cont) + Sebashell | 20 kHz | 13 mm probe | 50 | sec | 100% | 10% | 1 | 5 | mm |
| 41 | FDC Immersion(cont) + Sebashell | 20 kHz | 13 mm probe | 50 | sec | 100% | 10% | 1 | 5 | mm |
| 42 | Average of three rows above | 20 kHz | 13 mm probe | 50 | sec | 100% | 10% | 3 | 5 | mm |
| 43 | FDC Immersion(cont) + Water | 20 kHz | 13 mm probe | 50 | sec | 100% | 10% | 1 | 5 | mm |
| 44 | FDC Immersion(cont) + Water | 20 kHz | 13 mm probe | 50 | sec | 100% | 10% | 1 | 5 | mm |
| 45 | FDC Immersion(cont) + Water | 20 kHz | 13 mm probe | 50 | sec | 100% | 10% | 1 | 5 | mm |
| 46 | Average of three rows above | 20 kHz | 13 mm probe | 50 | sec | 100% | 10% | 3 | 5 | mm |
| 47 | FDC Immersion(cont) + Ethanol | 20 kHz | 13 mm probe | 50 | sec | 100% | 10% | 1 | 5 | mm |
| 48 | FDC Immersion(cont) + Ethanol | 20 kHz | 13 mm probe | 50 | sec | 100% | 10% | 1 | 5 | mm |
| 49 | FDC Immersion(cont) + Ethanol | 20 kHz | 13 mm probe | 50 | sec | 100% | 10% | 1 | 5 | mm |
| 50 | Average of three rows above | 20 kHz | 13 mm probe | 50 | sec | 100% | 10% | 2 | 5 | mm |
| 51 | FDC Immersion(cont) + DMSO | 20 kHz | 13 mm probe | 50 | sec | 100% | 10% | 1 | 5 | mm |
| 52 | FDC Immersion(cont) + DMSO | 20 kHz | 13 mm probe | 50 | sec | 100% | 10% | 1 | 5 | mm |
| 53 | Average of two rows above | 20 kHz | 13 mm probe | 50 | sec | 100% | 10% | 2 | 5 | mm |
| 54 | FDC Immersion(cont) + SS(twice) | 20 kHz | 13 mm probe | 50 | sec | 100% | 10% | 1 | 5 | mm |
| 55 | FDC Immersion(cont) + SS(twice) | 20 kHz | 13 mm probe | 50 | sec | 100% | 10% | 1 | 5 | mm |
| 56 | FDC Immersion(cont) + SS(twice) | 20 kHz | 13 mm probe | 50 | sec | 100% | 10% | 1 | 5 | mm |
| 57 | Average of three rows above | 20 kHz | 13 mm probe | 50 | sec | 100% | 10% | 3 | 5 | mm |
| 58 | FDC Immersion(cont) + SS(twice, | 20 kHz | 13 mm probe | 50 | sec | 100% | 10% | 1 | 5 | mm |
| | recycle) | | | | | | | | | |
| 59 | FDC Immersion(cont) + SS(twice, | 20 kHz | 13 mm probe | 50 | sec | 100% | 10% | 1 | 5 | mm |
| | recycle) | | | | | | | | | |
| 60 | Average of two rows above | 20 kHz | 13 mm probe | 50 | sec | 100% | 10% | 2 | 5 | mm |
| 61 | FDC Immersion (cont, 130 W) + | 20 kHz | 13 mm probe | 50 | sec | 100% | 20% | 1 | 5 | mm |
| | Sebashell | | | | | | | | | |
| 62 | FDC Immersion(cont, | 20 kHz | 13 mm probe | 50 | sec | 100% | 20% | 1 | 5 | mm |
| | 130 W) + Sebashell | | | | | | | | | |
| 63 | FDC Immersion(cont, | 20 kHz | 13 mm probe | 50 | sec | 100% | 20% | 1 | 5 | mm |
| | 130 W) + Sebashell | | | | | | | | | |
| 64 | FDC Immersion(cont, | 20 kHz | 13 mm probe | 50 | sec | 100% | 20% | 3 | 5 | mm |
| | 130 W) + Sebashell | | | | | | | | | |
| 65 | FDC Immersion(cont, | 20 kHz | 13 mm probe | 50 | sec | 100% | 20% | 1 | 5 | mm |
| | 130 W) + Acetone | | | | | | | | | |
| 66 | FDC Immersion(cont, 130 W) + | 20 kHz | 13 mm probe | 35 | sec | 100% | 20% | 1 | 5 | mm |
| | SS in 33 C. | | | | | | | | | |
| 67 | FDC Immersion(cont, 130 W) + | 20 kHz | 13 mm probe | 35 | sec | 100% | 20% | 1 | 5 | mm |
| | SS in 33 C. | | | | | | | | | |
| 68 | FDC Immersion(cont, 130 W) + | 20 kHz | 13 mm probe | 47 | sec | 100% | 20% | 1 | 5 | mm |
| | SS in 33 C. | | | | | | | | | |
| 69 | FDC Immersion(cont, 130 W) + | 20 kHz | 13 mm probe | 30 | sec | 100% | 20% | 1 | 5 | mm |
| | SS in 33 C. | | | | | | | | | |
| 70 | FDC Immersion(cont, 130 W, | 20 kHz | 13 mm probe | 35 | sec | 100% | 20% | 1 | 5 | mm |
| | human) in 33 C. | | | | | | | | | |
| 71 | FDC Immersion(cont, 130 W, | 20 kHz | 13 mm probe | 30 | sec | 100% | 20% | 1 | 5 | mm |
| | human w/hair) in 33 C. | | | | | | | | | |
| 72 | FDC Immersion(cont, 600 W, | 20 kHz | 13 mm probe | 47 | sec | 100% | 10% | 1 | 5 | mm |
| | human) | | | | | | | | | |
| 73 | Massage(rabbit ear) | | | 4 | min | 100% | weak | 1 | 0 | mm |
| 74 | FDC Immersion(cont, 130 W, | 20 kHz | 13 mm probe | 30 | sec | 100% | 20% | 1 | 5 | mm |
| | rabbit) | | | | | | | | | |
| 75 | FDC Immersion(cont, 130 W) + SS | 20 kHz | 13 mm probe | 30 | sec | 100% | 20% | 1 | 5 | mm |
| 76 | FDC Immersion(cont, 130 W) + SS | 20 kHz | 13 mm probe | 30 | sec | 100% | 20% | 1 | 5 | mm |
| 77 | Sonoprep Immersion(cont) + SS | 55 kHz | 10 mm probe | 40 | sec | 100% | 12 W RMS | 1 | 7.5 | mm |
| 78 | Sonoprep Immersion(cont) + SS | 55 kHz | 10 mm probe | 50 | sec | 100% | 12 W RMS | 1 | 7.5 | mm |
| 79 | Sonoprep Immersion(cont) + SS | 55 kHz | 10 mm probe | 25 | sec | 100% | 12 W RMS | 1 | 7.5 | mm |
| 80 | Sonoprep Immersion(cont) + SS | 55 kHz | 10 mm probe | 45 | sec | 100% | 12 W RMS | 1 | 7.5 | mm |
| 81 | FDC Immersion(cont, 130 W) + SS | 20 kHz | 13 mm probe | 30 | sec | 100% | 20% | 1 | 8 | mm |
| 82 | FDC Immersion(cont, 130 W) + SS | 20 kHz | 13 mm probe | 30 | sec | 100% | 20% | 1 | 8 | mm |
| 83 | FDC Immersion(cont, 130 W) + SS | 40 kHz | 13 mm probe | 30 | sec | 100% | 28% | 1 | 8 | mm |
| 84 | FDC Immersion(cont, 130 W) + SS | 40 kHz | 13 mm probe | 30 | sec | 100% | 28% | 1 | 8 | mm |
| 85 | FDC Immersion(cont, 130 W) + SS | 40 kHz | 13 mm probe | 30 | sec | 100% | 28% | 1 | 8 | mm |
| 86 | Average of three rows above | 40 kHZ | 13 mm probe | 30 | sec | 100% | 28% | 3 | 8 | mm |
| 87 | FDC Immersion(cont, 130 W) + SS | 40 kHZ | 13 mm probe | 30 | sec | 100% | 20% | 1 | 8 | mm |
| 88 | FDC Immersion(cont, 130 W) + SS | 40 kHZ | 13 mm probe | 30 | sec | 100% | 20% | 1 | 8 | mm |
| 89 | FDC Immersion(cont, 130 W) + SS | 40 kHZ | 13 mm probe | 30 | sec | 100% | 20% | 1 | 8 | mm |
| 90 | Average of three rows above | 40 kHZ | 13 mm probe | 30 | sec | 100% | 20% | 3 | 8 | mm |
| 91 | FDC Immersion(cont, 130 W) + SS | 40 kHZ | 13 mm probe | 30 | sec | 100% | 28% | 1 | 5 | mm |
| 92 | FDC Immersion(cont, 130 W) + SS | 40 kHZ | 13 mm probe | 30 | sec | 100% | 28% | 1 | 5 | mm |
| 93 | Average of two rows above | 40 kHZ | 13 mm probe | 30 | sec | 100% | 28% | 2 | 5 | mm |
| 94 | FDC Immersion(cont, 130 W) + SS | 40 kHZ | 13 mm probe | 30 | sec | 100% | 28% | 1 | 5 | mm |
| 95 | FDC Immersion(cont, 130 W) + SS | 40 kHZ | 13 mm probe | 30 | sec | 100% | 28% | 1 | 5 | mm |
| 96 | Average of two rows above | 40 kHZ | 13 mm probe | 30 | sec | 100% | 20% | 2 | 5 | mm |
| 97 | FDC Immersion(cont, 130 W) + SS | 40 kHZ | 13 mm probe | 30 | sec | 100% | 20% | 1 | 8 | mm |
| 98 | FDC Immersion(cont, 130 W) + SS | 40 kHZ | 13 mm probe | 30 | sec | 100% | 20% | 1 | 8 | mm |
| 99 | Average of two rows above | 40 kHZ | 13 mm probe | 30 | sec | 100% | 20% | 2 | 8 | mm |
| 100 | FDC Immersion(cont, 130 W) + SS | 40 kHZ | 13 mm probe | 30 | sec | 100% | 28% | 1 | 8 | mm |
| 101 | FDC Immersion(cont, 130 W) + SS | 40 kHZ | 13 mm probe | 30 | sec | 100% | 28% | 1 | 8 | mm |
| 102 | Average of two rows above | 40 kHZ | 13 mm probe | 30 | sec | 100% | 28% | 2 | 8 | mm |
| 103 | FDC Immersion(cont, 130 W) + SS | 40 kHZ | 13 mm probe | 30 | sec | 100% | 20% | 1 | 8 | mm |
| 104 | FDC Immersion(cont, 130 W) + SS | 40 kHZ | 13 mm probe | 30 | sec | 100% | 20% | 1 | 8 | mm |
| 105 | Average of two rows above | 40 kHZ | 13 mm probe | 30 | sec | 100% | 20% | 2 | 8 | mm |
| 106 | FDC Immersion(cont, 130 W) + SS | 40 kHZ | 13 mm probe | 30 | sec | 100% | 28% | 1 | 8 | mm |
| 107 | FDC Immersion(cont, 130 W) + SS | 40 kHZ | 13 mm probe | 30 | sec | 100% | 28% | 1 | 8 | mm |
| 108 | Average of two rows above | 40 kHZ | 13 mm probe | 30 | sec | 100% | 28% | 2 | 8 | mm |
| 109 | Average of rows 83-85, 100-101, | 40 kHZ | 13 mm probe | 30 | sec | 100% | 20% | 7 | 8 | mm |
| | and 106-107 above | | | | | | | | | |
| 110 | Average of rows 87-89, 97-98, | 40 kHZ | 13 mm probe | 30 | sec | 100% | 35% | 7 | 8 | mm |
| | and 103-104 above | | | | | | | | | |
| 111 | FDC Immersion(cont, 130 W) + SS | 40 kHZ | 13 mm probe | 30 | sec | 100% | 35% | 1 | 8 | mm |
| 112 | FDC Immersion(cont, 130 W) + SS | 40 kHZ | 13 mm probe | 30 | sec | 100% | 35% | 1 | 8 | mm |
| 113 | Average of two rows above | 40 kHZ | 13 mm probe | 30 | sec | 100% | 35% | 2 | 8 | mm |
| 114 | THC Immersion(cont, 130 W), ABS, | 20 kHZ | 13 mm probe | 30 | sec | 100% | 20% | 1 | 8 mm (10 mm) | |
| | 20 mm | | | | | | | | | |
| 115 | THC Immersion(cont, 130 W) + SS, | 20 kHZ | 13 mm probe | 30 | sec | 100% | 20% | 1 | 8 mm (10 mm) | |
| | ABS, 20 mm | | | | | | | | | |
| 116 | 20 mm ABS Immersion(cont, | 40 kHZ | 13 mm probe | 30 | sec | 100% | 28% | 1 | about 8 | mm |
| | 130 W) + SS | | | | | | | | | |
| 117 | 18 mm ABS Immersion(cont, | 40 kHz | 13 mm probe | 30 | sec | 100% | 28% | 1 | about 8 | mm |
| | 130 W) + SS | | | | | | | | | |
| 118 | 18 mm Al Immersion(cont, | 40 kHz | 13 mm probe | 30 | sec | 100% | 28% | 1 | about 8 | mm |
| | 130 W) + SS | | | | | | | | | |
| 119 | 16 mm ABS Immersion(cont, | 40 kHz | 13 mm probe | 30 | sec | 100% | 28% | 1 | about 8 | mm |
| | 130 W) + SS | | | | | | | | | |
| 120 | 16 mm Al Immersion(cont, | 40 kHz | 13 mm probe | 30 | sec | 100% | 28% | 1 | about 8 | mm |
| | 130 W) + SS | | | | | | | | | |
| 121 | 20 mm ABS Immersion(cont, | 40 kHz | 13 mm probe | 30 | sec | 100% | 28% | 1 | about 8 | mm |
| | 130 W) + SS | | | | | | | | | |
| 122 | 20 mm ABS Immersion(cont, | 40 kHz | 13 mm probe | 30 | sec | 100% | 28% | 1 | about 8 | mm |
| | 130 W) + SS | | | | | | | | | |
| 123 | Average of two rows above | 40 kHz | 13 mm probe | 30 | sec | 100% | 28% | 2 | about 8 | mm |
| 124 | 16 mm Al Immersion(cont, | 40 kHz | 13 mm probe | 30 | sec | 100% | 28% | 1 | about 8 | mm |
| | 130 W) + SS | | | | | | | | | |
| 125 | 16 mm Al Immersion(cont, | 40 kHz | 13 mm probe | 30 | sec | 100% | 28% | 1 | about 8 | mm |
| | 130 W) + SS | | | | | | | | | |
| 126 | Average of two rows above | 40 kHz | 13 mm probe | 30 | sec | 100% | 28% | 2 | about 8 | mm |
| 127 | 16 mm Al Immersion(cont, | 40 kHz | 13 mm probe | 30 | sec | 100% | 28% | 1 | about 8 | mm |
| | 130 W) + SS | | | | | | | | | |
| 128 | 16 mm Al Immersion(cont, | 40 kHz | 13 mm probe | 30 | sec | 100% | 28% | 1 | about 8 | mm |
| | 130 W) + SS | | | | | | | | | |
| 129 | 16 mm Al Immersion(cont, | 40 kHz | 13 mm probe | 30 | sec | 100% | 28% | 1 | about 8 | mm |
| | 130 W) + SS | | | | | | | | | |
| 130 | Average of three rows above | 40 kHz | 13 mm probe | 30 | sec | 100% | 28% | 3 | about 8 | mm |
| 131 | 16 mm ABS Immersion(cont, | 40 kHz | 13 mm probe | 30 | sec | 100% | 28% | 1 | about 8 | mm |
| | 130 W) + SS | | | | | | | | | |
| 132 | 16 mm ABS Immersion(cont, | 40 kHz | 13 mm probe | 30 | sec | 100% | 28% | 1 | about 8 | mm |
| | 130 W) + SS | | | | | | | | | |
| 133 | 16 mm ABS Immersion(cont, | 40 kHz | 13 mm probe | 30 | sec | 100% | 28% | 1 | about 8 | mm |
| | 130 W) + SS | | | | | | | | | |
| 134 | Average of three rows above | 40 kHz | 13 mm probe | 30 | sec | 100% | 28% | 3 | about 8 | mm |
| 135 | THC Immersion(cont, 130 W), ABS, | 40 kHz | 13 mm probe | 60 | sec | 100% | 28% | 1 | about 8 | mm |
| | 20 mm | | | | | | | | | |
| 136 | THC Immersion(cont, 130 W), ABS, | 40 kHz | 13 mm probe | 60 | sec | 100% | 28% | 1 | about 8 | mm |
| | 20 mm | | | | | | | | | |
| 137 | THC Immersion(cont, 130 W), ABS, | 40 kHz | 13 mm probe | 60 | sec | 100% | 28% | 1 | about 8 | mm |
| | 20 mm | | | | | | | | | |
| 138 | THC Immersion(cont, 130 W), ABS, | 40 kHz | 13 mm probe | 60 | sec | 100% | 28% | 1 | about 8 | mm |
| | 20 mm | | | | | | | | | |
| 139 | THC Immersion(cont, 130 W), ABS, | 40 kHz | 13 mm probe | 60 | sec | 100% | 28% | 1 | about 8 | mm |
| | 20 mm | | | | | | | | | |
| 140 | THC Immersion(cont, 130 W), ABS, | 40 kHz | 13 mm probe | 60 | sec | 100% | 28% | 1 | about 8 | mm |
| | 20 mm | | | | | | | | | |
| 141 | Average of six rows above | 40 kHz | 13 mm probe | 60 | sec | 100% | 28% | 6 | about 8 | mm |
| 142 | FDC Immersion(cont, 130 W) | 40 kHz | 13 mm probe | 30 | sec | 100% | 28% | 1 | about 8 | mm |
| 143 | FDC Immersion(cont, 130 W) | 40 kHz | 13 mm probe | 30 | sec | 100% | 28% | 1 | about 8 | mm |
| 144 | FDC Immersion(cont, 130 W) | 40 kHz | 13 mm probe | 30 | sec | 100% | 28% | 1 | about 8 | mm |
| 145 | FDC Immersion(cont, 130 W) | 40 kHz | 13 mm probe | 30 | sec | 100% | 28% | 1 | about 8 | mm |
| 146 | Average of four rows above | 40 kHz | 13 mm probe | 30 | sec | 100% | 28% | 4 | about 8 | mm |
| 147 | FDC Immersion(cont, 130 W) + SS- | 40 kHz | 13 mm probe | 30 | sec | 100% | 28% | 1 | about 8 | mm |
| | DIA | | | | | | | | | |
| 148 | FDC Immersion(cont, 130 W) + SS- | 40 kHz | 13 mm probe | 30 | sec | 100% | 28% | 1 | about 8 | mm |
| | DIA | | | | | | | | | |
| 149 | Average of two rows above | 40 kHz | 13 mm probe | 30 | sec | 100% | 28% | 2 | about 8 | mm |
| 150 | FDC Immersion(cont, 130 W) + SS | 40 kHz | 13 mm probe | 30 | sec | 100% | 28% | 1 | about 8 | mm |
| | OD75 | | | | | | | | | |
| 151 | FDC Immersion(cont, 130 W) + SS | 40 kHz | 13 mm probe | 30 | sec | 100% | 28% | 1 | about 8 | mm |
| | OD75 | | | | | | | | | |
| 152 | FDC Immersion(cont, 130 W) + SS | 40 kHz | 13 mm probe | 30 | sec | 100% | 28% | 1 | about 8 | mm |
| | OD75 | | | | | | | | | |
| 153 | FDC Immersion(cont, 130 W) + SS | 40 kHz | 13 mm probe | 30 | sec | 100% | 28% | 1 | about 8 | mm |
| | OD75 | | | | | | | | | |
| 154 | Average of four rows above | 40 kHz | 13 mm probe | 30 | sec | 100% | 28% | 4 | about 8 | mm |
| 155 | FDC Immersion(cont, 130 W) + SS | 40 kHz | 13 mm probe | 30 | sec | 100% | 28% | 1 | about 8 | mm |
| | OD125 | | | | | | | | | |
| 156 | FDC Immersion(cont, 130 W) + SS | 40 kHz | 13 mm probe | 30 | sec | 100% | 28% | 1 | about 8 | mm |
| | OD125 | | | | | | | | | |
| 157 | FDC Immersion(cont, 130 W) + SS | 40 kHz | 13 mm probe | 30 | sec | 100% | 28% | 1 | about 8 | mm |
| | OD125 | | | | | | | | | |
| 158 | FDC Immersion(cont, 130 W) + SS | 40 kHz | 13 mm probe | 30 | sec | 100% | 28% | 1 | about 8 | mm |
| | OD125 | | | | | | | | | |
| 159 | Average of four rows above | 40 kHz | 13 mm probe | 30 | sec | 100% | 28% | 4 | about 8 | mm |
| 160 | THC Immersion(cont, 130 W), Al, | 40 kHz | 13 mm probe | 60 | sec | 100% | 28% | 1 | about 8 | mm |
| | 15 mm, Al-plate | | | | | | | | | |
| 161 | THC Immersion(cont, 130 W), Al, | 40 kHz | 13 mm probe | 60 | sec | 100% | 28% | 1 | about 8 | mm |
| | 16 mm, Al-plate | | | | | | | | | |
| 162 | THC Immersion(cont, 130 W), Al, | 40 kHz | 13 mm probe | 60 | sec | 100% | 28% | 1 | about 8 | mm |
| | 16 mm, Al-plate | | | | | | | | | |
| 163 | Average of three rows above | 40 kHz | 13 mm probe | 60 | sec | 100% | 28% | 3 | about 8 | mm |
| 164 | THC Immersion(cont, 130 W), Al, | 40 kHz | 13 mm probe | 60 | sec | 100% | 28% | 1 | about 8 | mm |
| | 16 mm, Al-plate | | | | | | | | | |
| 165 | THC Immersion(cont, 130 W), Al, | 40 kHz | 13 mm probe | 60 | sec | 100% | 28% | 1 | about 8 | mm |
| | 16 mm, Al-plate | | | | | | | | | |
| 166 | Average of two rows above | 40 kHz | 13 mm probe | 60 | sec | 100% | 28% | 2 | about 8 | mm |
| 167 | FDC-top only, Imm. (cont, 130 W), | 40 kHz | 13 mm probe | 60 | sec | 100% | 28% | 1 | about 8 | mm |
| | glass 16 mm, Al plate | | | | | | | | | |
| 168 | FDC-top only, Imm. (cont, 130 W), | 40 kHz | 13 mm probe | 60 | sec | 100% | 28% | 1 | about 8 | mm |
| | glass 16 mm, Al plate | | | | | | | | | |
| 169 | Average of two rows above | 40 kHz | 13 mm probe | 60 | sec | 100% | 28% | 2 | about 8 | mm |
| 170 | THC Imm. (cont, 130 W), ABS, | 40 kHz | 13 mm probe | 60 | sec | 100% | 28% | 1 | about 6-7 | mm |
| | 20 mm, H2O, Receiver H2O | | | | | | | | | |
| 171 | THC Imm. (cont, 130 W), ABS, | 40 kHz | 13 mm probe | 60 | sec | 100% | 28% | 1 | about 6-7 | mm |
| | 20 mm, H2O, Receiver H2O | | | | | | | | | |
| 172 | Average of two rows above | 40 kHz | 13 mm probe | 60 | sec | 100% | 28% | 2 | about 6-7 | mm |
| 173 | THC Imm. (cont, 130 W), Al, | 40 kHz | 13 mm probe | 60 | sec | 100% | 28% | 1 | about 8 | mm |
| | 16 mm, H2O, Receiver | | | | | | | | | |
| 174 | THC Imm. (cont, 130 W), Al, | 40 kHz | 13 mm probe | 60 | sec | 100% | 28% | 1 | about 8 | mm |
| | 16 mm, H2O, Receiver | | | | | | | | | |
| 175 | Average of two rows above | 40 kHz | 13 mm probe | 60 | sec | 100% | 28% | 2 | about 8 | mm |
| 176 | THC Imm. (cont, 130 W), ABS, | 40 kHz | 13 mm probe | 60 | sec | 100% | 28% | 1 | about 8 | mm |
| | 20 mm, Air Receiver | | | | | | | | | |
| 177 | THC Imm. (cont, 130 W), ABS, | 40 kHz | 13 mm probe | 60 | sec | 100% | 28% | 1 | about 8 | mm |
| | 20 mm, Air Receiver | | | | | | | | | |
| 178 | THC Imm. (cont, 130 W), ABS, | 40 kHz | 13 mm probe | 60 | sec | 100% | 28% | 1 | about 8 | mm |
| | 20 mm, Air Receiver | | | | | | | | | |
| 179 | THC Imm. (cont, 130 W), ABS, | 40 kHz | 13 mm probe | 60 | sec | 100% | 28% | 1 | about 8 | mm |
| | 20 mm, Air Receiver | | | | | | | | | |
| 180 | Average of four rows above | 40 kHz | 13 mm probe | 60 | sec | 100% | 28% | 4 | about 8 | mm |
| 181 | THC Imm. (cont, 130 W), ABS, | 40 kHz | 13 mm probe | 60 | sec | 100% | 28% | 1 | about 8 | mm |
| | 20 mm, H2O, Receiver, beef T-bone, | | | | | | | | | |
| | with cartilige | | | | | | | | | |
| 182 | THC Imm, (cont, 130 W), ABS, | 40 kHz | 13 mm probe | 60 | sec | 100% | 28% | 1 | about 8 | mm |
| | 20 mm, H2O, Receiver, beef T-bone, | | | | | | | | | |
| | with cartilige | | | | | | | | | |
| 183 | Average of two rows above | 40 kHz | 13 mm probe | 60 | sec | 100% | 28% | 2 | about 8 | mm |
| 184 | THC Imm. (cont, 130 W), ABS, | 40 kHz | 13 mm probe | 60 | sec | 100% | 28% | 1 | about 8 | mm |
| | 20 mm, H2O, Receiver, beef T-bone, | | | | | | | | | |
| | w/o cartilige | | | | | | | | | |
| 185 | THC Imm. (cont, 130 W), ABS, | 40 kHz | 13 mm probe | 60 | sec | 100% | 28% | 1 | about 8 | mm |
| | 20 mm, H2O, Receiver, beef T-bone, | | | | | | | | | |
| | w/o cartilige | | | | | | | | | |
| 186 | Average of two rows above | 40 kHz | 13 mm probe | 60 | sec | 100% | 28% | 2 | about 8 | mm |
| 187 | THC Imm. (cont, 13 W), ABS, | 40 kHz | 13 mm probe | 60 | sec | 100% | 28% | 1 | about 8 | mm |
| | 20 mm, H2O, Receiver, pig bone, | | | | | | | | | |
| | w/o cartilige | | | | | | | | | |
| 188 | THC Imm. (cont, 130 W), ABS, | 40 kHz | 13 mm probe | 60 | sec | 100% | 28% | 1 | about 8 | mm |
| | 20 mm, H2O, Receiver, pig bone, | | | | | | | | | |
| | w/o cartilige | | | | | | | | | |
| 189 | Average of two rows above | 40 kHz | 13 mm probe | 60 | sec | 100% | 28% | 2 | about 8 | mm |
| 190 | THC Imm. (cont, 130 W), FDC, | 40 kHz | 13 mm probe | 60 | sec | 100% | 28% | 1 | about 8 | mm |
| | 16 mm, H2O, Receiver, pig bore, | | | | | | | | | |
| | w/o cartilige | | | | | | | | | |
| 191 | THC Imm. (cont, 130 W), FDC, | 40 kHz | 13 mm probe | 60 | sec | 100% | 28% | 1 | about 8 | mm |
| | 16 mm, H2O, Receiver, pig bone, | | | | | | | | | |
| | w/o cartilige | | | | | | | | | |
| 192 | Average of two rows above | 40 kHz | 13 mm probe | 60 | sec | 100% | 28% | 2 | about 8 | mm |
| 193 | FDC Imm. (cont, 130 W), 75OD | 40 kHz | 13 mm probe | 60 | sec | 100% | 28% | 1 | about 8 | mm |
| 194 | FDC Imm. (cont, 130 W), 75OD | 40 kHz | 13 mm probe | 60 | sec | 100% | 28% | 1 | about 8 | mm |
| 195 | Average of two rows above | 40 kHz | 13 mm probe | 60 | sec | 100% | 28% | 2 | about 8 | mm |
| 196 | FDC Imm. (cont, 130 W), 20 mm US | 40 kHz | 13 mm probe | 60 | sec | 100% | 28% | 1 | about 8 | mm |
| | F78 | | | | | | | | | |
| 197 | FDC Imm. (cont 130 W), 20 mm US | 40 kHz | 13 mm probe | 60 | sec | 100% | 28% | 1 | about 8 | mm |
| | F78 | | | | | | | | | |
| 198 | Average of two rows above | 40 kHz | 13 mm probe | 60 | sec | 100% | 28% | 2 | about 8 | mm |
| 199 | FDC Imm. (cont, 130 W), 20 mm | 40 kHz | 13 mm probe | 60 | sec | 100% | 28% | 1 | about 8 | mm |
| | horn | | | | | | | | | |
| 200 | FDC Imm. (cont, 130 W), 20 mm | 40 kHz | 13 mm probe | 60 | sec | 100% | 28% | 1 | about 8 | mm |
| | horn | | | | | | | | | |
| 201 | Average of two rows above | 40 kHz | 13 mm probe | 60 | sec | 100% | 28% | 2 | about 8 | mm |
| 202 | FDC Imm. (cont, 130 W), 20 mm | 40 kHz | 13 mm probe | 60 | sec | 100% | 28% | 1 | about 8 | mm |
| | horn | | | | | | | | | |
| 203 | FDC Imm. (cont, 130 W), 20 mm | 40 kHz | 13 mm probe | 60 | sec | 100% | 28% | 1 | about 8 | mm |
| | horn | | | | | | | | | |
| 204 | Average of two rows above | 40 kHz | 13 mm probe | 60 | sec | 100% | 28% | 2 | about 8 | mm |
| 205 | FDC Imm. (cont, 130 W), 13 mm | 40 kHz | 13 mm probe | 60 | sec | 100% | 20% | 1 | 12 | mm |
| | horn | | | | | | | | | |
| 206 | FDC Imm. (cont, 130 W), 13 mm | 40 kHz | 13 mm probe | 60 | sec | 100% | 20% | 1 | 12 | mm |
| | horn | | | | | | | | | |
| 207 | Average of two rows above | 40 kHz | 13 mm probe | 60 | sec | 100% | 20% | 2 | 12 | mm |
| 208 | FDC Imm. (cont, 130 W), 13 mm | 40 kHz | 13 mm probe | 60 | sec | 100% | 20% | 1 | 15 | mm |
| | horn | | | | | | | | | |
| 209 | FDC Imm. (cont, 130 W), 13 mm | 40 kHz | 13 mm probe | 60 | sec | 100% | 20% | 1 | 15 | mm |
| | horn | | | | | | | | | |
| 210 | Average of two rows above | 40 kHz | 13 mm probe | 60 | sec | 100% | 20% | 2 | 15 | mm |

| | A | I | J | K | L | M | N | O |
|---|---|---|---|---|---|---|---|---|
| 1 | Description | SS Volume | Laser | Total rate | Std. Dev. | SG rate | Std. Dev. | Deep SG |
| 2 | Massage | 1.0 ml | 50 J/cm2, 30 ms | | | | | |
| 3 | FDC Immersion | 1.5 ml | 50 J/cm2, 30 ms | | | | | |
| 4 | FDC Immersion | 1.5 ml | 50 J/cm2, 30 ms | | | | | |
| 5 | FDC Immersion | 1.5 ml | 50 J/cm2, 30 ms | 46.7 | | 16.7 | | |
| 6 | FDC Immersion | 1.5 ml | 50 J/cm2, 30 ms | 60 | | 20 | | |
| 7 | FDC Immersion | 1.5 ml | 50 J/cm2, 30 ms | 61 | | | | |
| 8 | FDC Immersion | 1.5 ml | 50 J/cm2, 30 ms | | | | | |
| 9 | FDC Immersion | 1.5 ml | 50 J/cm2, 30 ms | | | | | |
| 10 | FDC Immersion | 1.5 ml | 50 J/cm2, 30 ms | | | | | |
| 11 | Direct contact | 1.0 ml | 50 J/cm2, 30 ms | | | | | |
| 12 | FDC Immersion(cont, 130 W) | 1.5 ml | 50 J/cm2, 30 ms | 61.9 | | 38.1 | | 9.5 |
| 13 | FDC Immersion(cont, 130 W) | 1.7 ml | 50 J/cm2, 30 ms | 50.0 | | 20.0 | | 5.0 |
| 14 | FDC Immersion(cont, 130 W) | 1.7 ml | 50 J/cm2, 30 ms | 61.9 | | 47.6 | | 4.8 |
| 15 | Average of three rows above | 1.5 ml | 50 J/cm2, 30 ms | 57.9 | 6.9 | 35.2 | 14.0 | 6.4 |
| 16 | FDC Immersion(cont) | 1.5 ml | 50 J/cm2, 30 ms | 53.6 | | 25.0 | | 21.5 |
| 17 | FDC Immersion (cont) | 1.5 ml | 50 J/cm2, 30 ms | 56.2 | | 18.8 | | 12.6 |
| 18 | Average of two rows above | 1.5 ml | 50 J/cm2, 30 ms | 54.9 | 1.8 | 21.9 | 4.4 | 17.0 |
| 19 | FDC Immersion(cont) | 1.7 ml | 50 J/cm2, 30 ms | 77 | | 7.7 | | |
| 20 | FDC Immersion(cont) | 1.7 ml | 50 J/cm2, 30 ms | 55 | | 35 | | 0.0 |
| 21 | Average of two rows above | 1.7 ml | 50 J/cm2, 30 ms | 66.0 | 15.6 | 21.4 | 19.3 | 0.0 |
| 22 | FDC Immersion(cont, 130 W) + Ace | 1.7 ml | 50 J/cm2, 30 ms | 65 | | 41 | | 17.6 |
| 23 | FDC Immersion + Acetane | 1.5 ml | 50 J/cm2, 30 ms | 57.9 | | 15.8 | | 0.0 |
| 24 | FDC Immersions + Acetone | 1.5 ml | 50 J/cm2, 30 ms | 56 | | 28 | | 28.0 |
| 25 | FDC Immersion + Acetone | 1.5 ml | 50 J/cm2, 30 ms | 68.2 | | 36.4 | | 32.0 |
| 26 | FDC Immersion + Acetone + Vacuum | 1.5 ml | 50 J/cm2, 30 ms | 60.9 | | 39.1 | | 26.2 |
| 27 | FDC Immersion(cont) + Acetane | 1.7 ml | 50 J/cm2, 30 ms | 62 | | 52 | | 0.0 |
| 28 | FDC Immersion(Aca, | 1.0 ml | 50 J/cm2, 30 ms | 65 | | 45 | | 5.0 |
| | cont) + Massage | | | | | | | |
| 29 | FDC Immersian(cont) + Ace | 1.7 ml | 50 J/cm2, 30 ms | 70.0 | | 50.0 | | 30.0 |
| 30 | FDC Immersion(cont) + Ace | 1.7 ml | 50 J/cm2, 30 ms | 71.0 | | 55.0 | | 30.3 |
| 31 | FDC Immersion(cont) + Ace | 1.7 ml | 50 J/cm2, 30 ms | 65.0 | | 41.0 | | 0.0 |
| 32 | FDC Immersion(cont) + Ace | 1.7 ml | 50 J/cm2, 30 ms | 52.0 | | 31.0 | | 23.3 |
| 33 | Average of four rows above | 1.7 ml | 50 J/cm2, 30 ms | 64.5 | 8.7 | 44.3 | 10.6 | 20.9 |
| 34 | FDC Immersion(cont) + Isopropanol | 1.7 ml | 50 J/cm2, 30 ms | 70.0 | | 41.0 | | 23.4 |
| 35 | FDC Immersion(cont) + Isopropanol | 1.7 ml | 50 J/cm2, 30 ms | 65.0 | | 41.0 | | 13.5 |
| 36 | FDC Immersion(cont) + Isopropanol | 1.7 ml | 50 J/cm2, 30 ms | 76.0 | | 52.0 | | 14.0 |
| 37 | FDC Immersion(cont) + Isopropanol | 1.7 ml | 50 J/cm2, 30 ms | 72.0 | | 31.0 | | 20.8 |
| 38 | Average of four rows above | 1.7 ml | 50 J/cm2, 30 ms | 70.8 | 4.6 | 41.3 | 8.6 | 17.9 |
| 39 | FDC Immersion(cont) + Sebashell | 1.7 ml | 50 J/cm2, 30 ms | 86.0 | | 42.0 | | 26.5 |
| 40 | FDC Immersion(cont) + Sebashell | 1.7 ml | 50 J/cm2, 30 ms | 72.0 | | 42.0 | | 16.8 |
| 41 | FDC Immersion(cont) + Sebashell | 1.7 ml | 50 J/cm2, 30 ms | 90.0 | | 65.0 | | 50.1 |
| 42 | Average of three rows above | 1.7 ml | 50 J/cm2, 30 ms | 82.7 | 9.5 | 49.7 | 13.3 | 31.1 |
| 43 | FDC Immersion(cont) + Water | 1.7 ml | 50 J/cm2, 30 ms | 79.0 | | 42.0 | | 16.8 |
| 44 | FDC Immersion(cont) + Water | 1.7 ml | 50 J/cm2, 30 ms | 61.0 | | 52.0 | | 15.1 |
| 45 | FDC Immersion(cont) + Water | 1.7 ml | 50 J/cm2, 30 ms | 40.0 | | 20.0 | | 10.0 |
| 46 | Average of three rows above | 1.7 ml | 50 J/cm2, 30 ms | 60.0 | 19.5 | 38.0 | 16.4 | 14.0 |
| 47 | FDC Immersion(cont) + Ethanol | 1.7 ml | 50 J/cm2, 30 ms | 82.0 | | 38.0 | | 25.5 |
| 48 | FDC Immersion(cont) + Ethanol | 1.7 ml | 50 J/cm2, 30 ms | 67.0 | | 22.0 | | 5.5 |
| 49 | FDC Immersion(cont) + Ethanol | 1.7 ml | 50 J/cm2, 30 ms | 54.0 | | 10.0 | | 10.0 |
| 50 | Average of three rows above | 1.7 ml | 50 J/cm2, 30 ms | 67.7 | 14.0 | 23.3 | 14.0 | 13.7 |
| 51 | FDC Immersion(cont) + DMSO | 1.7 ml | 50 J/cm2, 30 ms | 60.0 | | 45.0 | | 0.0 |
| 52 | FDC Immersion(cont) + DMSO | 1.7 ml | 50 J/cm2, 30 ms | 63.0 | | 33.0 | | 7.3 |
| 53 | Average of two rows above | 1.7 ml | 50 J/cm2, 30 ms | 61.5 | 2.1 | 39.0 | 8.5 | 3.6 |
| 54 | FDC Immersion(cont) + SS(twice) | 1.7 ml | 50 J/cm2, 30 ms | 96.0 | | 33.0 | | 33.0 |
| 55 | FDC Immersion(cont) + SS(twice) | 1.7 ml | 50 J/cm2, 30 ms | 93.0 | | 79.0 | | 50.6 |
| 56 | FDC Immersion(cont) + SS(twice) | 1.7 ml | 50 J/cm2, 30 ms | 94.0 | | 84.0 | | 33.6 |
| 57 | Average of three rows above | 1.7 ml | 50 J/cm2, 30 ms | 94.3 | 1.5 | 65.3 | 28.1 | 39.1 |
| 58 | FDC Immersion(cont) + SS(twice, | 1.7 ml | 50 J/cm2, 30 ms | 83 | | 59 | | 31.9 |
| | recycle) | | | | | | | |
| 59 | FDC Immersion(cont) + SS(twice, | 1.7 ml | 50 J/cm2, 30 ms | 91 | | 74 | | 39.2 |
| | recycle) | | | | | | | |
| 60 | Average of two rows above | 1.7 ml | 50 J/cm2, 30 ms | 87.0 | 5.7 | 66.5 | 10.6 | 35.5 |
| 61 | FDC Immersion (cont, 130 W) + | 1.7 ml | 50 J/cm2, 30 ms | 91.3 | | 63.2 | | 26.3 |
| | Sebashell | | | | | | | |
| 62 | FDC Immersion(cont, | 1.7 ml | 50 J/cm2, 30 ms | 100.0 | | 75.0 | | 12.5 |
| | 130 W) + Sebashell | | | | | | | |
| 63 | FDC Immersion(cont, | 1.7 ml | 50 J/cm2, 30 ms | 95.0 | | 55.0 | | 15.0 |
| | 130 W) + Sebashell | | | | | | | |
| 64 | FDC Immersion(cont, | 1.7 ml | 50 J/cm2, 30 ms | 95.4 | 4.4 | 64.4 | 10.1 | 17.9 |
| | 130 W) + Sebashell | | | | | | | |
| 65 | FDC Immersion(cont, | 1.7 ml | 50 J/cm2, 30 ms | 96.2 | | 70.0 | | 10.0 |
| | 130 W) + Acetone | | | | | | | |
| 66 | FDC Immersion(cont, 130 W) + SS in | 1.7 ml | 50 J/cm2, 30 ms | 100.0 | | 68.0 | | 25.8 |
| | 33 C. | | | | | | | |
| 67 | FDC Immersion(cont, 130 W) + SS in | 1.7 ml | 50 J/cm2, 30 ms | 100.0 | | 54.0 | | 23.2 |
| | 33 C. | | | | | | | |
| 68 | FDC Immersion(cont, 130 W) + SS in | 1.7 ml | 50 J/cm2, 30 ms | 82.0 | | 35.0 | | 15.1 |
| | 33 C. | | | | | | | |
| 69 | FDC Immersion(cont, 130 W) + SS in | 1.7 ml | 50 J/cm2, 30 ms | 80.0 | | 40.0 | | 15.2 |
| | 33 C. | | | | | | | |
| 70 | FDC Immersion(cont, 130 W, | 1.7 ml | 50 J/cm2, 30 ms | 44.0 | | 4/? | | |
| | human) in 33 C. | | | | | | | |
| 71 | FDC Immersion(cont, 130 W, | 1.7 ml | 50 J/cm2, 30 ms | 12.5 | | 0.0 | | |
| | human w/hair) in 33 C. | | | | | | | |
| 72 | FDC Immersion(cont, 600 W, | 1.7 ml | 50 J/cm2, 30 ms | 2/6 biopsies | | 2 (possible) | | |
| | human) | | | | | | | |
| 73 | Massage(rabbit ear) | 1.0 ml | 50 J/cm2, 30 ms | | | | | |
| 74 | FDC Immersion(cont, 130 W, | 1.7 ml | 50 J/cm2, 30 ms | | | | | |
| | rabbit) | | | | | | | |
| 75 | FDC Immersion(cont, 130 W) + SS | 1.7 ml | 50 J/cm2, 30 ms | 100.0 | | 50.0 | | 16.5 |
| 76 | FDC Immersion(cont, 130 W) + SS | 1.7 ml | 50 J/cm2, 30 ms | 78.0 | | 61.0 | | 27.5 |
| 77 | Sonoprep Immersion(cont) + SS | 1.0 ml | 50 J/cm2, 30 ms | 77.0 | | 15.0 | | 0.0 |
| 78 | Sonoprep Immersion(cont) + SS | 1.0 ml | 50 J/cm2, 30 ms | 93.0 | | 60.0 | | 6.6 |
| 79 | Sonoprep Immersion(cont) + SS | 1.0 ml | 50 J/cm2, 30 ms | 36.0 | | 9.0 | | 9.0 |
| 80 | Sonoprep Immersion(cont) + SS | 1.0 ml | 50 J/cm2, 30 ms | 75.0 | | 33.0 | | 0.0 |
| 81 | FDC Immersion(cont, 130 W) + SS | 2.0 ml | 50 J/cm2, 30 ms | 100.0 | | 25.0 | | 0.0 |
| 82 | FDC Immersion(cont, 130 W) + SS | 2.0 ml | 50 J/cm2, 30 ms | 90.0 | | 10.0 | | 0.0 |
| 83 | FDC Immersion(cont, 130 W) + SS | 2.0 ml | 50 J/cm2, 30 ms | 85.7 | | 40.0 | | 15.0 |
| 84 | FDC Immersion(cont, 130 W) + SS | 2.0 ml | 50 J/cm2, 30 ms | 100.0 | | 64.3 | | 50.0 |
| 85 | FDC Immersion(cont, 130 W) + SS | 2.0 ml | 50 J/cm2, 30 ms | 100.0 | | 60.0 | | 19.8 |
| 86 | Average of three rows above | 2.0 ml | 50 J/cm2, 30 ms | 95.2 | 8.2 | 54.8 | 13.0 | 28.3 |
| 87 | FDC Immersion(cont, 130 W) + SS | 2.0 ml | 50 J/cm2, 30 ms | 68.4 | | 42.1 | | 21.1 |
| 88 | FDC Immersion(cont, 130 W) + SS | 2.0 ml | 50 J/cm2, 30 ms | 50.0 | | 21.4 | | 14.3 |
| 89 | FDC Immersion(cont, 130 W) + SS | 2.0 ml | 50 J/cm2, 30 ms | 84.2 | | 50.0 | | 50.0 |
| 90 | Average of three rows above | 2.0 ml | 50 J/cm2, 30 ms | 67.5 | 17.1 | 37.8 | 14.8 | 28.4 |
| 91 | FDC Immersion(cont, 130 W) + SS | 2.0 ml | 50 J/cm2, 30 ms | 100.0 | | 37.5 | | 25.0 |
| 92 | FDC Immersion(cont, 130 W) + SS | 2.0 ml | 50 J/cm2, 30 ms | 84.2 | | 54.5 | | 18.2 |
| 93 | Average of two rows above | 2.0 ml | 50 J/cm2, 30 ms | 92.1 | 11.2 | 46.0 | 12.1 | 21.6 |
| 94 | FDC Immersion(cont, 130 W) + SS | 2.0 ml | 50 J/cm2, 30 ms | 60.0 | | 37.5 | | 12.5 |
| 95 | FDC Immersion(cont, 130 W) + SS | 2.0 ml | 50 J/cm2, 30 ms | 81.3 | | 28.6 | | 14.3 |
| 96 | Average of two rows above | 2.0 ml | 50 J/cm2, 30 ms | 70.6 | 15.0 | 33.0 | 6.3 | 13.4 |
| 97 | FDC Immersion(cont, 130 W) + SS | 2.0 ml | 50 J/cm2, 30 ms | 56.7 | | 36.4 | | 18.2 |
| 98 | FDC Immersion(cont, 130 W) + SS | 2.0 ml | 50 J/cm2, 30 ms | 86.4 | | 60.0 | | 30.0 |
| 99 | Average of two rows above | 2.0 ml | 50 J/cm2, 30 ms | 76.5 | 13.9 | 48.2 | 16.7 | 24.1 |
| 100 | FDC Immersion(cont, 130 W) + SS | 2.0 ml | 50 J/cm2, 30 ms | 80.0 | | 42.1 | | 31.6 |
| 101 | FDC Immersion(cont, 130 W) + SS | 2.0 ml | 50 J/cm2, 30 ms | 83.3 | | 70.0 | | 40.0 |
| 102 | Average of two rows above | 2.0 ml | 50 J/cm2, 30 ms | 81.7 | 2.4 | 56.1 | 19.7 | 35.8 |
| 103 | FDC Immersion(cont, 130 W) + SS | 2.0 ml | 50 J/cm2, 30 ms | 75.0 | | 16.7 | | 8.3 |
| 104 | FDC Immersion(cont, 130 W) + SS | 2.0 ml | 50 J/cm2, 30 ms | 100.0 | | 57.9 | | 36.8 |
| 105 | Average of two rows above | 2.0 ml | 50 J/cm2, 30 ms | 87.5 | 17.7 | 37.3 | 29.2 | 22.6 |
| 106 | FDC Immersion(cont, 130 W) + SS | 2.0 ml | 50 J/cm2, 30 ms | 76.5 | | 28.6 | | 7.1 |
| 107 | FDC Immersion(cont, 130 W) + SS | 2.0 ml | 50 J/cm2, 30 ms | 100.0 | | 40.0 | | 40.0 |
| 108 | Average of two rows above | 2.0 ml | 50 J/cm2, 30 ms | 88.2 | 16.6 | 34.3 | 8.1 | 23.6 |
| 109 | Average of rows 83-85, 100-101, | 2.0 ml | 50 J/cm2, 30 ms | 75.8 | 16.2 | 40.6 | 17.0 | 25.5 |
| | and 106-107 above | | | | | | | |
| 110 | Average of rows 87-89, 97-98, | 2.0 ml | 50 J/cm2, 30 ms | 89.4 | 10.4 | 49.3 | 15.4 | 29.1 |
| | and 103-104 above | | | | | | | |
| 111 | FDC Immersion(cont, 130 W) + SS | 2.0 ml | 50 J/cm2, 30 ms | Sebashell | 66.7 | | 4.8 | |
| 112 | FDC Immersion(cont, 130 W) + SS | 2.0 ml | 50 J/cm2, 30 ms | 50.0 | | 5.0 | | 0.0 |
| 113 | Average of two rows above | 2.0 ml | 50 J/cm2, 30 ms | 58.3 | 11.8 | 4.9 | 0.2 | 2.4 |
| 114 | THC Immersion(cont, 130 W), ABS, | 5.0 ml | 50 J/cm2, 30 ms | 61.1 | | 22.2 | | 0.0 |
| | 20 mm | | | | | | | |
| 115 | THC Immersion(cont, 130 W) + SS, | 5.0 ml | 50 J/cm2, 30 ms | 67.6 | | 33.3 | | 10.0 |
| | ABS, 20 mm | | | | | | | |
| 116 | 20 mm ABS Immersion(cont, | 4.0 ml | 50 J/cm2, 30 ms | 72.0 | | 11.0 | | 6.0 |
| | 130 W) + SS | | | | | | | |
| 117 | 18 mm ABS Immersion(cont, | 3.2 ml | 50 J/cm2, 30 ms | 81.0 | | No SGs | | |
| | 130 W) + SS | | | | | | | |
| 118 | 18 mm Al Immersion(cont, | 3.2 ml | 50 J/cm2, 30 ms | 50.0 | | No SGs | | |
| | 130 W) + SS | | | | | | | |
| 119 | 16 mm ABS Immersion(cont, | 2.5 ml | 50 J/cm2, 30 ms | 100.0 | | 6.0 | | |
| | 130 W) + SS | | | | | | | |
| 120 | 16 mm Al Immersion(cont, | 2.5 ml | 50 J/cm2, 30 ms | 100.0 | | 22.0 | | 22.0 |
| | 130 W) + SS | | | | | | | |
| 121 | 20 mm ABS Immersion(cont, | 4.0 ml | 50 J/cm2, 30 ms | 86.4 | | 36.4 | | 31.8 |
| | 130 W) + SS | | | | | | | |
| 122 | 20 mm ABS Immersion(cont, | 4.0 ml | 50 J/cm2, 30 ms | 61.1 | | 37.5 | | 31.3 |
| | 130 W) + SS | | | | | | | |
| 123 | Average of two rows above | 4.0 ml | 50 J/cm2, 30 ms | 73.7 | 17.9 | 36.9 | 0.8 | 31.5 |
| 124 | 16 mm Al Immersion(cont, | 2.5 ml | 50 J/cm2, 30 ms | 100.0 | | 30.8 | | 15.4 |
| | 130 W) + SS | | | | | | | |
| 125 | 16 mm Al Immersion(cont, | 2.5 ml | 50 J/cm2, 30 ms | 100.0 | | 16.7 | | 0.0 |
| | 130 W) + SS | | | | | | | |
| 126 | Average of two rows above | 2.5 ml | 50 J/cm2, 30 ms | 100.0 | 0.0 | 23.7 | 10.0 | 7.7 |
| 127 | 16 mm Al Immersion(cont, | 2.5 ml | 50 J/cm2, 30 ms | 84.2 | | 16.7 | | 5.6 |
| | 130 W) + SS | | | | | | | |
| 128 | 16 mm Al Immersion(cont, | 2.5 ml | 50 J/cm2, 30 ms | 81.3 | | 56.3 | | 6.3 |
| | 130 W) + SS | | | | | | | |
| 129 | 16 mm Al Immersion(cont, | 2.5 ml | 50 J/cm2, 30 ms | 85.7 | | 35.7 | | 0.0 |
| | 130 W) + SS | | | | | | | |
| 130 | Average of three rows above | 2.5 ml | 50 J/cm2, 30 ms | 83.7 | 2.3 | 36.2 | 19.8 | 3.9 |
| 131 | 16 mm ABS Immersion(cont, | 2.5 ml | 50 J/cm2, 30 ms | 100.0 | | 33.3 | | 11.1 |
| | 130 W) + SS | | | | | | | |
| 132 | 16 mm ABS Immersion(cont, | 2.5 ml | 50 J/cm2, 30 ms | 100.0 | | 50.0 | | 8.3 |
| | 130 W) + SS | | | | | | | |
| 133 | 16 mm ABS Immersion(cont, | 2.5 ml | 50 J/cm2, 30 ms | 71.4 | | 40.0 | | 10.0 |
| | 130 W) + SS | | | | | | | |
| 134 | Average of three rows above | 2.5 ml | 50 J/cm2, 30 ms | 90.5 | 16.5 | 41.1 | 8.4 | 9.8 |
| 135 | THC Immersion(cont, 130 W), ABS, | 4.5 ml | 50 J/cm2, 30 ms | 82.4 | | 26.7 | | 6.7 |
| | 20 mm | | | | | | | |
| 136 | THC Immersion(cont, 130 W), ABS, | 4.5 ml | 50 J/cm2, 30 ms | 70.4 | | 33.3 | | 4.8 |
| | 20 mm | | | | | | | |
| 137 | THC Immersion(cont, 130 W), ABS, | 3.5 ml | 50 J/cm2, 30 ms | 100.0 | | 47.1 | | 5.9 |
| | 20 mm | | | | | | | |
| 138 | THC Immersion(cont, 130 W), ABS, | 3.5 ml | 50 J/cm2, 30 ms | 77.8 | | 47.6 | | 23.8 |
| | 20 mm | | | | | | | |
| 139 | THC Immersion(cont, 130 W), ABS, | 3.5 ml | 50 J/cm2, 30 ms | 80.8 | | 38.5 | | 0.0 |
| | 20 mm | | | | | | | |
| 140 | THC Immersion(cont, 130 W), ABS, | 3.5 ml | 50 J/cm2, 30 ms | 88.9 | | 33.3 | | 0.0 |
| | 20 mm | | | | | | | |
| 141 | Average of six rows above | 4.5 ml | 50 J/cm2, 30 ms | 83.4 | 10.1 | 37.7 | 8.3 | 6.9 |
| 142 | FDC Immersion(cont, 130 W) | 2.0 ml | 50 J/cm2, 30 ms | 100.0 | | 41.7 | | 16.7 |
| 143 | FDC Immersion(cont, 130 W) | 2.0 ml | 50 J/cm2, 30 ms | 64.3 | | 28.6 | | 0.0 |
| 144 | FDC Immersion(cont, 130 W) | 2.0 ml | 50 J/cm2, 30 ms | 90.5 | | 66.7 | | 13.3 |
| 145 | FDC Immersion(cont, 130 W) | 2.0 ml | 50 J/cm2, 30 ms | 100.0 | | 54.5 | | 18.2 |
| 146 | Average of four rows above | 2.0 ml | 50 J/cm2, 30 ms | 88.7 | 16.9 | 47.9 | 16.4 | 12.0 |
| 147 | FDC Immersion(cont, 130 W) + SS- | 2.0 ml | 50 J/cm2, 30 ms | 91.3 | | 44.4 | | 0.0 |
| | DIA | | | | | | | |
| 148 | FDC Immersion(cont, 130 W) + SS- | 2.0 ml | 50 J/cm2, 30 ms | 84.0 | | 31.8 | | 0.0 |
| | DIA | | | | | | | |
| 149 | Average of two rows above | 2.0 ml | 50 J/cm2, 30 ms | 83.9 | 14.4 | 44.9 | 19.2 | 21.1 |
| 150 | FDC Immersion(cont, 130 W) + SS | 2.0 ml | 50 J/cm2, 30 ms | 91.3 | | 53.8 | | 38.5 |
| | OD75 | | | | | | | |
| 151 | FDC Immersion(cont, 130 W) + SS | 2.0 ml | 50 J/cm2, 30 ms | 81.3 | | 21.4 | | 0.0 |
| | OD75 | | | | | | | |
| 152 | FDC Immersion(cont, 130 W) + SS | 2.0 ml | 50 J/cm2, 30 ms | 92.3 | | 33.3 | | 0.0 |
| | OD75 | | | | | | | |
| 153 | FDC Immersion(cont, 130 W) + SS | 2.0 ml | 50 J/cm2, 30 ms | 80.0 | | 0.0 | | 0.0 |
| | OD75 | | | | | | | |
| 154 | Average of four rows above | 2.0 ml | 50 J/cm2, 30 ms | 86.2 | 6.5 | 27.2 | 22.5 | 9.6 |
| 155 | FDC Immersion(cont, 130 W) + SS | 2.0 ml | 50 J/cm2, 30 ms | 90.5 | | 42.9 | | 23.8 |
| | OD125 | | | | | | | |
| 156 | FDC Immersion(cont, 130 W) + SS | 2.0 ml | 50 J/cm2, 30 ms | 72.2 | | 43.8 | | 31.3 |
| | OD125 | | | | | | | |
| 157 | FDC Immersion(cont, 130 W) + SS | 2.0 ml | 50 J/cm2, 30 ms | 89.5 | | 57.9 | | 47.4 |
| | OD125 | | | | | | | |
| 158 | FDC Immersion(cont, 130 W) + SS | 2.0 ml | 50 J/cm2, 30 ms | 92.3 | | 20.0 | | 20.0 |
| | OD125 | | | | | | | |
| 159 | Average of four rows above | 2.0 ml | 50 J/cm2, 30 ms | 86.1 | 9.3 | 41.1 | 15.7 | 30.6 |
| 160 | THC Immersion(cont, 130 W), Al, | 4.5 ml | 50 J/cm2, 30 ms | 47.4 | | 15.8 | | 0.0 |
| | 15 mm, Al-plate | | | | | | | |
| 161 | THC Immersion(cont, 130 W), Al, | 4.5 ml | 50 J/cm2, 30 ms | 55.6 | | 5.6 | | 0.0 |
| | 16 mm, Al-plate | | | | | | | |
| 162 | THC Immersion(cont, 130 W), Al, | 3.5 ml | 50 J/cm2, 30 ms | 33.3 | | 0.0 | | 0.0 |
| | 16 mm, Al-plate | | | | | | | |
| 163 | Average of three rows above | 4.5 ml | 50 J/cm2, 30 ms | 45.4 | 11.2 | 7.1 | 8.0 | 0.0 |
| 164 | THC Immersion(cont, 130 W), Al, | 4.5 ml | 50 J/cm2, 30 ms | 46.2 | | 0.0 | | 0.0 |
| | 16 mm, Al-plate | | | | | | | |
| 165 | THC Immersion(cont, 130 W), Al, | 4.5 ml | 50 J/cm2, 30 ms | 76.9 | | 23.1 | | 0.0 |
| | 16 mm, Al-plate | | | | | | | |
| 166 | Average of two rows above | 4.5 ml | 50 J/cm2, 30 ms | 61.5 | 21.8 | 11.5 | 16.3 | 0.0 |
| 167 | FDC-top only, Imm. (cont, 130 W), | 4.5 ml | 50 J/cm2, 30 ms | 93.8 | | 31.3 | | 0.0 |
| | glass 16 mm, Al plate | | | | | | | |
| 168 | FDC-top only, Imm. (cont, 130 W), | 4.5 ml | 50 J/cm2, 30 ms | 95.2 | | 61.9 | | 4.8 |
| | glass 16 mm, Al plate | | | | | | | |
| 169 | Average of two rows above | 4.5 ml | 50 J/cm2, 30 ms | 94.5 | 1.1 | 46.6 | 21.7 | 2.4 |
| 170 | THC Imm. (cont, 130 W), ABS, 20 mm, | 4.5 ml | 50 J/cm2, 30 ms | 87.5 | | 62.5 | | 50.0 |
| | H2O, Receiver H2O | | | | | | | |
| 171 | THC Imm. (cont, 130 W), ABS, 20 mm, | 4.5 ml | 50 J/cm2, 30 ms | 90.9 | | 66.7 | | 41.7 |
| | H2O, Receiver H2O | | | | | | | |
| 172 | Average of two rows above | 4.5 ml | 50 J/cm2, 30 ms | 89.2 | 2.4 | 64.6 | 2.9 | 45.8 |
| 173 | THC Imm. (cont, 130 W), Al, 16 mm, | 4.5 ml | 50 J/cm2, 30 ms | 87.5 | | 50.0 | | 18.8 |
| | H2O, Receiver | | | | | | | |
| 174 | THC Imm. (cont, 130 W), Al, 16 mm, | 4.5 ml | 50 J/cm2, 30 ms | 93.8 | | 62.5 | | 18.8 |
| | H2O, Receiver | | | | | | | |
| 175 | Average of two rows above | 4.5 ml | 50 J/cm2, 30 ms | 90.6 | 4.4 | 56.3 | 8.8 | 18.8 |
| 176 | THC Imm. (cont, 130 W), ABS, 20 mm, | 4.5 ml | 50 J/cm2, 30 ms | 82.4 | | 53.3 | | 40.0 |
| | Air Receiver | | | | | | | |
| 177 | THC Imm. (cont, 130 W), ABS, 20 mm, | 4.5 ml | 50 J/cm2, 30 ms | 87.5 | | 60.9 | | 34.8 |
| | Air Receiver | | | | | | | |
| 178 | THC Imm. (cont, 130 W), ABS, 20 mm, | 4.5 ml | 50 J/cm2, 30 ms | 68.2 | | 36.4 | | 4.5 |
| | Air Receiver | | | | | | | |
| 179 | THC Imm. (cont, 130 W), ABS, 20 mm, | 4.5 ml | 50 J/cm2, 30 ms | 100.0 | | 57.9 | | 26.3 |
| | Air Receiver | | | | | | | |
| 180 | Average of four rows above | 4.5 ml | 50 J/cm2, 30 ms | 84.5 | 13.2 | 52.1 | 10.9 | 26.4 |
| 181 | THC Imm. (cont, 130 W), ABS, 20 mm, | 4.5 ml | 50 J/cm2, 30 ms | 94.4 | | 61.1 | | 27.8 |
| | H2O, Receiver, beef T-bone, | | | | | | | |
| | with cartilige | | | | | | | |
| 182 | THC Imm, (cont, 130 W), ABS, 20 mm, | 4.5 ml | 50 J/cm2, 30 ms | 93.8 | | 50.0 | | 6.3 |
| | H2O, Receiver, beef T-bone, | | | | | | | |
| | with cartilige | | | | | | | |
| 183 | Average of two rows above | 4.5 ml | 50 J/cm2, 30 ms | 94.1 | 0.5 | 55.6 | 7.9 | 17.0 |
| 184 | THC Imm. (cont, 130 W), ABS, 20 mm, | 4.5 ml | 50 J/cm2, 30 ms | 86.4 | | 54.5 | | 36.4 |
| | H2O, Receiver, beef T-bone, | | | | | | | |
| | w/o cartilige | | | | | | | |
| 185 | THC Imm. (cont, 130 W), ABS, 20 mm, | 4.5 ml | 50 J/cm2, 30 ms | 80.0 | | 40.0 | | 6.7 |
| | H2O, Receiver, beef T-bone, | | | | | | | |
| | w/o cartilige | | | | | | | |
| 186 | Average of two rows above | 4.5 ml | 50 J/cm2, 30 ms | 83.2 | 4.5 | 47.3 | 10.3 | 21.5 |
| 187 | THC Imm. (cont, 13 W), ABS, 20 mm, | 4.5 ml | 50 J/cm2, 30 ms | 100.0 | | 50.0 | | 19.2 |
| | H2O, Receiver, pig bone, w/o | | | | | | | |
| | cartilige | | | | | | | |
| 188 | THC Imm. (cont, 130 W), ABS, 20 mm, | 4.5 ml | 50 J/cm2, 30 ms | 100.0 | | 66.7 | | 27.8 |
| | H2O, Receiver, pig bone, w/o | | | | | | | |
| | cartilige | | | | | | | |
| 189 | Average of two rows above | 4.5 ml | 50 J/cm2, 30 ms | 100.0 | 0.0 | 58.3 | 11.8 | 23.5 |
| 190 | THC Imm. (cont, 130 W), FDC, 16 mm, | 4.5 ml | 50 J/cm2, 30 ms | 87.5 | | 56.3 | | 31.3 |
| | H2O, Receiver, pig bore, w/o | | | | | | | |
| | cartilige | | | | | | | |
| 191 | THC Imm. (cont, 130 W), FDC, 16 mm, | 4.5 ml | 50 J/cm2, 30 ms | 92.3 | | 61.5 | | 30.8 |
| | H2O, Receiver, pig bone, w/o | | | | | | | |
| | cartilige | | | | | | | |
| 192 | Average of two rows above | 4.5 ml | 50 J/cm2, 30 ms | 89.9 | 3.4 | 58.9 | 3.7 | 31.0 |
| 193 | FDC Imm. (cont, 130 W), 75OD | 4.5 ml | 50 J/cm2, 30 ms | 85.7 | | 25.0 | | 10.0 |
| 194 | FDC Imm. (cont, 130 W), 75OD | 4.5 ml | 50 J/cm2, 30 ms | 70.8 | | 54.2 | | 45.8 |
| 195 | Average of two rows above | 4.5 ml | 50 J/cm2, 30 ms | 78.3 | 10.5 | 39.6 | 20.6 | 27.9 |
| 196 | FDC Imm. (cont, 130 W), 20 mm US | 4.5 ml | 50 J/cm2, 30 ms | 100.0 | | 50.0 | | 10.0 |
| | F78 | | | | | | | |
| 197 | FDC Imm. (cont 130 W), 20 mm US | 4.5 ml | 50 J/cm2, 30 ms | 100.0 | | 81.8 | | 72.7 |
| | F78 | | | | | | | |
| 198 | Average of two rows above | 4.5 ml | 50 J/cm2, 30 ms | 100.0 | 0.0 | 65.9 | 22.5 | 41.4 |
| 199 | FDC Imm. (cont, 130 W), 20 mm | 4.5 ml | 50 J/cm2, 30 ms | 89.2 | | 45.9 | | 15.6 |
| | horn | | | | | | | |
| 200 | FDC Imm. (cont, 130 W), 20 mm | 4.5 ml | 50 J/cm2, 30 ms | 68.5 | | 50.0 | | 26.1 |
| | horn | | | | | | | |
| 201 | Average of two rows above | 4.5 ml | 50 J/cm2, 30 ms | 78.9 | 14.6 | 48.4 | 2.2 | 20.9 |
| 202 | FDC Imm. (cont, 130 W), 20 mm | 4.5 ml | 50 J/cm2, 30 ms | 100.0 | | 80.0 | | 70.0 |
| | horn | | | | | | | |
| 203 | FDC Imm. (cont, 130 W), 20 mm | 4.5 ml | 50 J/cm2, 30 ms | 69.0 | | 42.9 | | 25.0 |
| | horn | | | | | | | |
| 204 | Average of two rows above | 4.5 ml | 50 J/cm2, 30 ms | 84.5 | 21.9 | 61.4 | 26.3 | 47.5 |
| 205 | FDC Imm. (cont, 130 W), 13 mm | 4.5 ml | 50 J/cm2, 30 ms | 92.9 | | 61.5 | | 23.1 |
| | horn | | | | | | | |
| 206 | FDC Imm. (cont, 130 W), 13 mm | 4.5 ml | 50 J/cm2, 30 ms | 78.6 | | 54.5 | | 18.2 |
| | horn | | | | | | | |
| 207 | Average of two rows above | 4.5 ml | 50 J/cm2, 30 ms | 85.7 | 10.1 | 58.0 | 4.9 | 20.6 |
| 208 | FDC Imm. (cont, 130 W), 13 mm | 4.5 ml | 50 J/cm2, 30 ms | 81.8 | | 27.3 | | 4.5 |
| | horn | | | | | | | |
| 209 | FDC Imm. (cont, 130 W), 13 mm | 4.5 ml | 50 J/cm2, 30 ms | 87.5 | | 43.8 | | 25.0 |
| | horn | | | | | | | |
| 210 | Average of two rows above | 4.5 ml | 50 J/cm2, 30 ms | 84.7 | 4.0 | 35.5 | 11.7 | 14.8 |

| | A | P | Q | | R |
|---|---|---|---|---|---|
| 1 | Description | Std. Dev. | MaxTemp | | Additional Description/comments |
| 2 | Massage | | | | |
| 3 | FDC Immersion | | | | |
| 4 | FDC Immersion | | | | |
| 5 | FDC Immersion | | 38 | C. | |
| 6 | FDC Immersion | | 43 | C. | |
| 7 | FDC Immersion | | 38 | C. | A little SGs |
| 8 | FDC Immersion | | | | |
| 9 | FDC Immersion | | | | A little SGs |
| 10 | FDC Immersion | | | | |
| 11 | Direct contact | | | | |
| 12 | FDC Immersion(cont, 130 W) | | 40.8 | C. | |
| 13 | FDC Immersion(cont, 130 W) | | 41.9 | C. | Little SGs |
| 14 | FDC Immersion(cont, 130 W) | | 40.8 | C. | |
| 15 | Average of three rows above | 2.7 | 40.8 | C. | |
| 16 | FDC Immersion(cont) | | 39.8 | C. | |
| 17 | FDC Immersion (cont) | | 41.5 | C. | |
| 18 | Average of two rows above | 6.3 | 39.8 | C. | |
| 19 | FDC Immersion(cont) | | 40.8 | C. | A little SGs |
| 20 | FDC Immersion(cont) | | 41.5 | C. | |
| 21 | Average of two rows above | | 41.5 | C. | |
| 22 | FDC Immersion(cont, 130 W) + Ace | | 41.3 | C. | Acetone + US 1 min(100% duty) |
| 23 | FDC Immersion + Acetane | | 38 | C. | Acetone 2 min |
| 24 | FDC Immersions + Acetone | | 43 | C. | Acetone + US 2 min |
| 25 | FDC Immersion + Acetone | | | | Acetone + US 4 min |
| 26 | FDC Immersion + Acetone + Vacuum | | | | Acetone + US 2 min + Vac dry 1 min |
| 27 | FDC Immersion(cont) + Acetane | | 41.5 | C. | Acetone + US 1 min(100% duty) |
| 28 | FDC Immersion(Aca, | | | | Acetone + US 1 min(100% duty) |
| | cont) + Massage | | | | |
| 29 | FDC Immersian(cont) + Ace | | 38.8 | C. | Acetone + US 1 min(100% duty) |
| 30 | FDC Immersion(cont) + Ace | | 38 | C. | Acetone + US 1 min(100% duty) |
| 31 | FDC Immersion(cont) + Ace | | 38 | C. | Acetone + US 1 min(100% duty) |
| 32 | FDC Immersion(cont) + Ace | | 38 | C. | Acetone + US 50 sec(100% duty) |
| 33 | Average of four rows above | 14.3 | 38.8 | C. | Acetone + US 1 min (100% duty) |
| 34 | FDC Immersion(cont) + Isopropanol | | 39 | C. | Isopropanol + US 1 min(100% duty) |
| 35 | FDC Immersion(cont) + Isopropanol | | 38 | C. | Isopropanol + US 1 min(100% duty) |
| 36 | FDC Immersion(cont) + Isopropanol | | 38 | C. | Isopropanol + US 1 min(100% duty) |
| 37 | FDC Immersion(cont) + Isopropanol | | 38 | C. | Isopropanol + US 50 sec(100% duty) |
| 38 | Average of four rows above | 4.9 | 39 | C. | Isopropanol + US 1 min(100% duty) |
| 39 | FDC Immersion(cont) + Sebashell | | 37 | C. | Sebashell + US 1 min(100% duty) |
| 40 | FDC Immersion(cont) + Sebashell | | 37 | C. | Sebashell + US 1 min(100% duty) |
| 41 | FDC Immersion(cont) + Sebashell | | 37 | C. | Sebashell + US 1 min(100% duty) |
| 42 | Average of three rows above | 17.1 | 37 | C. | Sebashell + US 1 min(100% duty) |
| 43 | FDC Immersion(cont) + Water | | 37 | C. | Water + US 1 min(100% duty) |
| 44 | FDC Immersion(cont) + Water | | 37 | C. | Water + US 1 min(100% duty) |
| 45 | FDC Immersion(cont) + Water | | 37 | C. | Water + US 1 min(100% duty) |
| 46 | Average of three rows above | 3.5 | 37 | C. | Water + US 1 min(100% duty) |
| 47 | FDC Immersion(cont) + Ethanol | | 39 | C. | Ethanol + US 1 min(100% duty) |
| 48 | FDC Immersion(cont) + Ethanol | | 38 | C. | Ethanol + US 50 sec(100% duty) |
| 49 | FDC Immersion(cont) + Ethanol | | 38 | C. | Ethanol + US 50 sec(100% duty) |
| 50 | Average of three rows above | 10.5 | 39 | C. | Ethanol + US 1 min(100% duty) |
| 51 | FDC Immersion(cont) + DMSO | | 39 | C. | DMSO + US 50 sec(100% duty) (45 C.) |
| 52 | FDC Immersion(cont) + DMSO | | 39 | C. | DMSO + US 50 sec(100% duty) (45 C.) |
| 53 | Average of two rows above | 5.1 | 39 | C. | DMSO + US 50 sec(100% duty) (45 C.) |
| 54 | FDC Immersion(cont) + SS(twice) | | 37 | C. | Sebashell + US 1 min(100% duty, twice) |
| 55 | FDC Immersion(cont) + SS(twice) | | 37 | C. | Sebashell + US 1 min(100% duty, twice) |
| 56 | FDC Immersion(cont) + SS(twice) | | 37 | C. | Sebashell + US 1 min(100% duty, twice) |
| 57 | Average of three rows above | 10.0 | 37 | C. | Sebashell + US 1 min(100% duty, twice) |
| 58 | FDC Immersion(cont) + SS(twice, | | 37 | C. | Sebashell + US 1 min(100% duty, twice) |
| | recycle) | | | | |
| 59 | FDC Immersion(cont) + SS(twice, | | 37 | C. | Sebashell + US 1 min(100% duty, twice) |
| | recycle) | | | | |
| 60 | Average of two rows above | 5.2 | 37 | C. | Sebashell + US 1 min(100% duty, twice) |
| 61 | FDC Immersion (cont, 130 W) + | | 40.5 | | Sebashell + US 45 sec(100% duty) |
| | Sebashell | | | | |
| 62 | FDC Immersion(cont, | | 40.5 | | Sebashell + US 45 sec(100% duty) |
| | 130 W) + Sebashell | | | | |
| 63 | FDC Immersion(cont, | | 40.5 | | Sebashell + US 45 sec(100% duty) |
| | 130 W) + Sebashell | | | | |
| 64 | FDC Immersion(cont, | 7.4 | 40.5 | | Sebashell + US 45 sec(100% duty) |
| | 130 W) + Sebashell | | | | |
| 65 | FDC Immersion(cont, | | 40.5 | | Acetone + US 1 min(100% duty) |
| | 130 W) + Acetone | | | | |
| 66 | FDC Immersion(cont, 130 W) + SS in | | 26.8-40.2 | | Sebashell + US 30 sec(100% duty) 28.3-39.8 C. |
| | 33 C. | | | | |
| 67 | FDC Immersion(cont, 130 W) + SS in | | 26.5-40.8 | | Sebashell + US 45 sec(100% duty) 26.4-40.4 C. |
| | 33 C. | | | | |
| 68 | FDC Immersion(cont, 130 W) + SS in | | 29.7-42.6 | | Sebashell + US 27 sec(100% duty) 29.4-41 C. |
| | 33 C. | | | | |
| 69 | FDC Immersion(cont, 130 W) + SS in | | 28-40.9 | | Sebashell + US 28 sec(100% duty) 28.8-40.4 C. |
| | 33 C. | | | | |
| 70 | FDC Immersion(cont, 130 W, | | 27-40.5 | | HumanSkin Temp: 30.5-36 |
| | human) in 33 C. | | | | |
| 71 | FDC Immersion(cont, 130 W, | | 29-42 | | Human Skin Temp: 29.4-35.2 |
| | human w/hair) in 33 C. | | | | |
| 72 | FDC Immersion(cont, 600 W, | | 21-40.7 | | |
| | human) | | | | |
| 73 | Massage(rabbit ear) | | | | |
| 74 | FDC Immersion(cont, 130 W, | | 21-40.7 | | Sebashell + US 30 sec(100% duty) 29.4-41 C. |
| | rabbit) | | | | |
| 75 | FDC Immersion(cont, 130 W) + SS | | 29.7-42.6 | | Sebashell + US 30 sec(100% duty) 29.4-41 C. |
| 76 | FDC Immersion(cont, 130 W) + SS | | 28-40.9 | | Sebashell + US 20 sec(100% duty) 28.8-40.4 C. |
| 77 | Sonoprep Immersion(cont) + SS | | 28-40.9 | | Sebashell + US 50 sec(100% duty) 28.8-40.4 C. |
| 78 | Sonoprep Immersion(cont) + SS | | 28-40.9 | | Sebashell + US 35 sec(100% duty) 28.8-40.4 C. |
| 79 | Sonoprep Immersion(cont) + SS | | 28-0.9 | | Sebashell + US 16 sec(100% duty) 28.8-40.9 C. |
| 80 | Sonoprep Immersion(cont) + SS | | 28-40.9 | | Sebashell + US 83 sec(100% duty) 28.8-40.4 C. |
| 81 | FDC Immersion(cont, 130 W) + SS | | | | |
| 82 | FDC Immersion(cont, 130 W) + SS | | | | |
| 83 | FDC Immersion(cont, 130 W) + SS | | | | |
| 84 | FDC Immersion(cont, 130 W) + SS | | | | |
| 85 | FDC Immersion(cont, 130 W) + SS | | | | |
| 86 | Average of three rows above | 19.0 | | | Sebashell + US 30 sec(100% duty) 29.4-41 C. |
| 87 | FDC Immersion(cont, 130 W) + SS | | | | |
| 88 | FDC Immersion(cont, 130 W) + SS | | | | |
| 89 | FDC Immersion(cont, 130 W) + SS | | | | |
| 90 | Average of three rows above | 19.0 | | | Sebashell + US 30 sec(100% duty) 29.4-41 C. |
| 91 | FDC Immersion(cont, 130 W) + SS | | | | |
| 92 | FDC Immersion(cont, 130 W) + SS | | | | |
| 93 | Average of two rows above | 4.8 | | | Sebashell + US 30 sec(100% duty) 29.4-41 C. |
| 94 | FDC Immersion(cont, 130 W) + SS | | | | |
| 95 | FDC Immersion(cont, 130 W) + SS | | | | |
| 96 | Average of two rows above | 1.3 | | | Sebashell + US 30 sec(100% duty) 29.4-41 C. |
| 97 | FDC Immersion(cont, 130 W) + SS | | | | Sebashell + US 30 sec(100% duty) 29.4-41 C. |
| 98 | FDC Immersion(cont, 130 W) + SS | | | | Sebashell + US 30 sec(100% duty) 29.4-41 C. |
| 99 | Average of two rows above | 8.4 | | | Sebashell + US 30 sec(100% duty) 29.4-41 C. |
| 100 | FDC Immersion(cont, 130 W) + SS | | | | Sebashell + US 30 sec(100% duty) 29.4-41 C. |
| 101 | FDC Immersion(cont, 130 W) + SS | | | | Sebashell + US 30 sec(100% duty) 29.4-41 C. |
| 102 | Average of two rows above | 6.0 | | | Sebashell + US 30 sec(100% duty) 29.4-41 C. |
| 103 | FDC Immersion(cont, 130 W) + SS | | | | Sebashell + US 30 sec(100% duty) 29.4-41 C. |
| 104 | FDC Immersion(cont, 130 W) + SS | | | | Sebashell + US 30 sec(100% duty) 29.4-41 C. |
| 105 | Average of two rows above | 20.2 | | | Sebashell + US 30 sec(100% duty) 29.4-41 C. |
| 106 | FDC Immersion(cont, 130 W) + SS | | | | Sebashell + US 30 sec(100% duty) 29.4-41 C. |
| 107 | FDC Immersion(cont, 130 W) + SS | | | | Sebashell + US 30 sec(100% duty) 29.4-41 C. |
| 108 | Average of two rows above | 23.2 | | | Sebashell + US 30 sec(100% duty) 29.4-41 C. |
| 109 | Average of rows 83-85, 100-101, | 14.4 | | | Sebashell + US 30 sec(100% duty) 29.4-41 C. |
| | and 106-107 above | | | | |
| 110 | Average of rows 87-89, 97-98, | 15.5 | | | Sebashell + US 30 sec(100% duty) 29.4-41 C. |
| | and 103-104 above | | | | |
| 111 | FDC Immersion(cont, 130 W) + SS | 0.0 | 4.8 | | Sebashell + US 30 sec(100% duty) 29.4-41 C. |
| 112 | FDC Immersion(cont, 130 W) + SS | | | | Sebashell + US 30 sec(100% duty) 29.4-41 C. |
| 113 | Average of two rows above | 3.4 | | | Sebashell + US 30 sec(100% duty) 29.4-41 C. |
| 114 | THC Immersion(cont, 130 W), ABS, | | | | |
| | 20 mm | | | | |
| 115 | THC Immersion(cont, 130 W) + SS, | | | | Sebashell + US 30 sec(100% duty) 20-25 C. |
| | ABS, 20 mm | | | | |
| 116 | 20 mm ABS Immersion(cont, | | 27-38 | | Sebashell + US 30 sec(100% duty) 24-36 C. |
| | 130 W) + SS | | | | |
| 117 | 18 mm ABS Immersion(cont, | | 27-41 | | Sebashell + US 30 sec(100% duty) 21-35 C. |
| | 130 W) + SS | | | | |
| 118 | 18 mm Al Immersion(cont, | | 27-37 | | Sebashell + US 30 sec(100% duty) 21-33 C. |
| | 130 W) + SS | | | | |
| 119 | 16 mm ABS Immersion(cont, | | 25-40 | | Sebashell + US 30 sec(100% duty) 23-39 C. |
| | 130 W) + SS | | | | |
| 120 | 16 mm Al Immersion(cont, | | 27-37 | | Sebashell + US 30 sec(100% duty) 21-33 C. |
| | 130 W) + SS | | | | |
| 121 | 20 mm ABS Immersion(cont, | | 28-40 | | Sebashell + US 30 sec(100% duty) 22-36 C. |
| | 130 W) + SS | | | | |
| 122 | 20 mm ABS Immersion(cont, | | 29-40 | | Sebashell + US 30 sec(100% duty) 26-38 C. |
| | 130 W) + SS | | | | |
| 123 | Average of two rows above | 0.4 | 28-40 | | Sebashell + US 30 sec(100% duty) 22-36 C. |
| 124 | 16 mm Al Immersion(cont, | | 28-39 | | Sebashell + US 30 sec(100% duty) 24-36 C. |
| | 130 W) + SS | | | | |
| 125 | 16 mm Al Immersion(cont, | | 28-38 | | Sebashell + US 30 sec(100% duty) 25-36 C. |
| | 130 W) + SS | | | | |
| 126 | Average of two rows above | 10.9 | 28-39 | | Sebashell + US 30 sec(100% duty) 24-36 C. |
| 127 | 16 mm Al Immersion(cont, | | 28-38 | | Sebashell + US 30 sec(100% duty) 22-33 C. |
| | 130 W) + SS | | | | |
| 128 | 16 mm Al Immersion(cont, | | 27-38 | | Sebashell + US 30 sec(100% duty) 21-34 C. |
| | 130 W) + SS | | | | |
| 129 | 16 mm Al Immersion(cont, | | 28-38 | | Sebashell + US 30 sec(100% duty) 21-33 C. |
| | 130 W) + SS | | | | |
| 130 | Average of three rows above | 3.4 | | | |
| 131 | 16 mm ABS Immersion(cont, | | 28-42 | | Sebashell + US 30 sec(100% duty) 22-38 C. |
| | 130 W) + SS | | | | |
| 132 | 16 mm ABS Immersion(cont, | | 27-40 | | Sebashell + US 30 sec(100% duty) 22-36 C. |
| | 130 W) + SS | | | | |
| 133 | 16 mm ABS Immersion(cont, | | 28-41 | | Sebashell + US 30 sec(100% duty) 22-36 C. |
| | 130 W) + SS | | | | |
| 134 | Average of three rows above | 1.4 | | | |
| 135 | THC Immersion(cont, 130 W), ABS, | | 19-38 | | |
| | 20 mm | | | | |
| 136 | THC Immersion(cont, 130 W), ABS, | | 20-41 | | |
| | 20 mm | | | | |
| 137 | THC Immersion(cont, 130 W), ABS, | | 20-37 | | |
| | 20 mm | | | | |
| 138 | THC Immersion(cont, 130 W), ABS, | | 24-36 | | |
| | 20 mm | | | | |
| 139 | THC Immersion(cont, 130 W), ABS, | | 20-38 | | |
| | 20 mm | | | | |
| 140 | THC Immersion(cont, 130 W), ABS, | | 25-42 | | |
| | 20 mm | | | | |
| 141 | Average of six rows above | 8.8 | | | |
| 142 | FDC Immersion(cont, 130 W) | | 22-33 | | |
| 143 | FDC Immersion(cont, 130 W) | | 26-39 | | |
| 144 | FDC Immersion(cont, 130 W) | | 19-39 | | |
| 145 | FDC Immersion(cont, 130 W) | | 19-36 | | |
| 146 | Average of four rows above | 8.3 | | | |
| 147 | FDC Immersion(cont, 130 W) + SS- | | 26-40 | | Sebashell + US 30 sec(100% duty) 22-35 C. |
| | DIA | | | | |
| 148 | FDC Immersion(cont, 130 W) + SS- | | 25-39 | | Sebashell + US 30 sec(100% duty) 23-38 C. |
| | DIA | | | | |
| 149 | Average of two rows above | 17.5 | | | Sebashell + US 30 sec(100% duty) 22-35 C. |
| 150 | FDC Immersion(cont, 130 W) + SS | | 26-40 | | Sebashell + US 30 sec(100% duty) 22-42 C. |
| | OD75 | | | | |
| 151 | FDC Immersion(cont, 130 W) + SS | | 29-41 | | Sebashell + US 30 sec(100% duty) 23-41 C. |
| | OD75 | | | | |
| 152 | FDC Immersion(cont, 130 W) + SS | | 23-40 | | Sebashell + US 30 sec(100% duty) 22-39 C. |
| | OD75 | | | | |
| 153 | FDC Immersion(cont, 130 W) + SS | | 25-41 | | Sebashell + US 30 sec(100% duty) 22-41 C. |
| | OD75 | | | | |
| 154 | Average of four rows above | 19.2 | | | Sebashell + US 30 sec(100% duty) 22-35 C. |
| 155 | FDC Immersion(cont, 130 W) + SS | | 25-40 | | Sebashell + US 30 sec(100% duty) 22-42 C. |
| | OD125 | | | | |
| 156 | FDC Immersion(cont, 130 W) + SS | | 29-41 | | Sebashell + US 30 sec(100% duty) 23-41 C. |
| | OD125 | | | | |
| 157 | FDC Immersion(cont, 130 W) + SS | | 23-40 | | Sebashell + US 30 sec(100% duty) 22-39 C. |
| | OD125 | | | | |
| 158 | FDC Immersion(cont, 130 W) + SS | | 25-41 | | Sebashell + US 30 sec(100% duty) 22-41 C. |
| | OD125 | | | | |
| 159 | Average of four rows above | 12.1 | | | Sebashell-US 30 sec(100% duty) 22-35 C. |
| 160 | THC Immersion(cont, 130 W), Al, | | 26-35 | | |
| | 15 mm, Al-plate | | | | |
| 161 | THC Immersion(cont, 130 W), Al, | | 24-36 | | |
| | 16 mm, Al-plate | | | | |
| 162 | THC Immersion(cont, 130 W), Al, | | 24-35 | | |
| | 16 mm, Al-plate | | | | |
| 163 | Average of three rows above | 0.0 | | | |
| 164 | THC Immersion(cont, 130 W), Al, | | 22-32 | | |
| | 16 mm, Al-plate | | | | |
| 165 | THC Immersion(cont, 130 W), Al, | | 22-31 | | |
| | 16 mm, Al-plate | | | | |
| 166 | Average of two rows above | 0.0 | | | |
| 167 | FDC-top only, Imm. (cont, 130 W), | | 26-40 | | Sebashell + US 30 sec(100% duty) 22-42 C. |
| | glass 16 mm, Al plate | | | | |
| 168 | FDC-top only, Imm. (cont, 130 W), | | 29-41 | | Sebashell + US 30 sec(100% duty) 23-41 C. |
| | glass 16 mm, Al plate | | | | |
| 169 | Average of two rows above | 3.4 | | | |
| 170 | THC Imm. (cont, 130 W), ABS, 20 mm, | | 22-37 | | Sebashell + US 30 sec(100% duty) 22-40 C. |
| | H2O, Receiver H2O | | | | |
| 171 | THC Imm. (cont, 130 W), ABS, 20 mm, | | 22-38 | | Sebashell + US 30 sec(100% duty) 23-40 C. |
| | H2O, Receiver H2O | | | | |
| 172 | Average of two rows above | 5.9 | | | |
| 173 | THC Imm. (cont, 130 W), Al, 16 mm, | | 22-32 | | |
| | H2O, Receiver | | | | |
| 174 | THC Imm. (cont, 130 W), Al, 16 mm, | | 22-31 | | |
| | H2O, Receiver | | | | |
| 175 | Average of two rows above | 0.0 | | | |
| 176 | THC Imm. (cont, 130 W), ABS, 20 mm, | | 30-38 | | Sebashell + US 30 sec(100% duty) 22-40 C. |
| | Air Receiver | | | | |
| 177 | THC Imm. (cont, 130 W), ABS, 20 mm, | | 30-38 | | Sebashell + US 30 sec(100% duty) 22-40 C. |
| | Air Receiver | | | | |
| 178 | THC Imm. (cont, 130 W), ABS, 20 mm, | | 30-38 | | Sebashell + US 30 sec(100% duty) 22-40 C. |
| | Air Receiver | | | | |
| 179 | THC Imm. (cont, 130 W), ABS, 20 mm, | | 31-41 | | Sebashell + US 30 sec(100% duty) 23-40 C. |
| | Air Receiver | | | | |
| 180 | Average of four rows above | 15.6 | | | |
| 181 | THC Imm. (cont, 130 W), ABS, 20 mm, | | 30-38 | | Sebashell + US 30 sec(100% duty) 22-40 C. |
| | H2O, Receiver, beef T-bone, | | | | |
| | with cartilige | | | | |
| 182 | THC Imm, (cont, 130 W), ABS, 20 mm, | | 30-38 | | Sebashell + US 30 sec(100% duty) 22-40 C. |
| | H2O, Receiver, beef T-bone, | | | | |
| | with cartilige | | | | |
| 183 | Average of two rows above | 15.2 | | | |
| 184 | THC Imm. (cont, 130 W), ABS, 20 mm, | | 30-38 | | Sebashell + US 30 sec(100% duty) 22-40 C. |
| | H2O, Receiver, beef T-bone, | | | | |
| | w/o cartilige | | | | |
| 185 | THC Imm. (cont, 130 W), ABS, 20 mm, | | 30-38 | | Sebashell + US 30 sec(100% duty) 22-40 C. |
| | H2O, Receiver, beef T-bone, | | | | |
| | w/o cartilige | | | | |
| 186 | Average of two rows above | 21.0 | | | |
| 187 | THC Imm. (cont, 13 W), ABS, 20 mm, | | 30-38 | | Sebashell + US 30 sec(100% duty) 22-40 C. |
| | H2O, Receiver, pig bone, w/o | | | | |
| | cartilige | | | | |
| 188 | THC Imm. (cont, 130 W), ABS, 20 mm, | | 30-38 | | Sebashell + US 30 sec(100% duty) 22-40 C. |
| | H2O, Receiver, pig bone, w/o | | | | |
| | cartilige | | | | |
| 189 | Average of two rows above | 6.0 | | | |
| 190 | THC Imm. (cont, 130 W), FDC, 16 mm, | | 30-38 | | Sebashell + US 30 sec(100% duty) 22-40 C. |
| | H2O, Receiver, pig bore, w/o | | | | |
| | cartilige | | | | |
| 191 | THC Imm. (cont, 130 W), FDC, 16 mm, | | 30-38 | | Sebashell + US 30 sec(100% duty) 22-40 C. |
| | H2O, Receiver, pig bone, w/o | | | | |
| | cartilige | | | | |
| 192 | Average of two rows above | 0.3 | | | |
| 193 | FDC Imm. (cont, 130 W), 75OD | | 30-38 | | Sebashell + US 30 sec(100% duty) 22-40 C. |
| 194 | FDC Imm. (cont, 130 W), 75OD | | 30-38 | | Sebashell + US 30 sec(100% duty) 22-40 C. |
| 195 | Average of two rows above | 25.3 | | | |
| 196 | FDC Imm. (cont, 130 W), 20 mm US | | 30-38 | | Sebashell + US 30 sec(100% duty) 22-40 C. |
| | F78 | | | | |
| 197 | FDC Imm. (cont 130 W), 20 mm US | | 30-38 | | Sebashell + US 30 sec(100% duty) 22-40 C. |
| | F78 | | | | |
| 198 | Average of two rows above | 44.4 | | | |
| 199 | FDC Imm. (cont, 130 W), 20 mm | | | | Sabashell + US 60 sec(100% duty) 22-33 C. |
| | horn | | | | |
| 200 | FDC Imm. (cont, 130 W), 20 mm | | | | Sebashell + US 60 sec(100% duty) 22-32 C. |
| | horn | | | | |
| 201 | Average of two rows above | 7.4 | | | |
| 202 | FDC Imm. (cont, 130 W), 20 mm | | | | Sebashell + US 60 sec(100% duty) 22-33 C. |
| | horn | | | | |
| 203 | FDC Imm. (cont, 130 W), 20 mm | | | | Sebashell + US 60 sec(100% duty) 22-30 C. |
| | horn | | | | |
| 204 | Average of two rows above | 31.8 | | | |
| 205 | FDC Imm. (cont, 130 W), 13 mm | | | | Sebashell + US 60 sec(100% duty) 22-40 C. |
| | horn | | | | |
| 206 | FDC Imm. (cont, 130 W), 13 mm | | | | Sebashell + US 60 sec(100% duty) 22-40 C. |
| | horn | | | | |
| 207 | Average of two rows above | 3.5 | | | |
| 208 | FDC Imm. (cont, 130 W), 13 mm | | | | Sebashell + US 60 sec(100% duty) 22-40 C. |
| | horn | | | | |
| 209 | FDC Imm. (cont, 130 W), 13 mm | | | | Sebashell + US 60 sec(100% duty) 22-40 C. |
| | horn | | | | |
| 210 | Average of two rows above | 14.5 | | | |

1. A method for treating a sebaceous gland disorder at a site on a patient's skin comprising the steps of:
a) administering a solvent to the site and applying immersion low frequency ultrasound to the site,
b) topically applying a formulation comprising nanoshell particles to the site and applying immersion low frequency ultrasound to the site, wherein the ultrasound delivers the nanoshell particles into the infundibula and sebaceous glands, and
c) irradiating the site at a wavelength that matches the absorption spectrum of the nanoshell particles.

2. A method for treating a sebaceous gland disorder at a site on a patient's skin comprising the steps of:
a) administering a solvent to the site and applying immersion low frequency ultrasound to the site,
b) topically applying a formulation comprising nanoshell particles to the site and applying at the site iontophoresis, low frequency ultrasound, massage, electroporation, or a combination thereof effective to deliver the nanoshell particles into the infundibula and sebaceous glands, and
c) irradiating the site at a wavelength that matches the absorption spectrum of the nanoshell particles.

3. The method of claim 1, wherein in step a, the ultrasound frequency is between about 20 kHz and about 100 kHz.

4. The method of claim 3, wherein the low frequency ultrasound is continuously applied to the skin for a period of time ranging from about 1 second to about 10 minutes, preferably between 2 seconds and 5 minutes, more preferably between 5 seconds and 1 minute.

5. The method of claim 1, wherein in step a, the tip of the ultrasonic horn is at least partially immersed into the solvent.

6. The method of claim 5, wherein the tip of the ultrasonic horn is about 1 mm to about 20 mm above the skin surface, preferably about 5 mm to about 15 mm above the skin surface.

7. The method of claim 1, wherein in step a, the low-frequency ultrasound causes the formation of microjets incident toward the skin surface.

8. The method of claim 7, wherein the microjets drive the solvent into the follicles.

9. The method of claim 7, wherein the microjets provide energy to the skin surface which heats the solvent and skin to a temperature sufficient to loosen, dislodge, destroy, or otherwise modify the blockage within a follicle.

10. The method of claim 2, wherein in step b immersion low frequency ultrasound is applied at the site.

11. The method of claim 1, wherein the low frequency ultrasound is pulsed or continuous.

12. The method of claim 11, wherein the low frequency ultrasound is continuously applied to the skin for a period of time ranging from about 1 second to about 10 minutes, preferably between 2 seconds and 5 minutes, more preferably between 5 seconds and 1 minute.

13. The method of claim 1, wherein the parameters and conditions of the immersion low frequency ultrasound in step a is the same as in step b.

14. The method of claim 1, wherein the solvent is selected from the group consisting of dimethylsulfoxide (DMSO), water, ethanol, isopropanol, acetone, and combinations thereof.

15. The method of claim 1, wherein steps a and b are consecutive.

16. The method of claim 1, wherein steps a and b are simultaneous.

17. The method of claim 1, wherein the sebaceous glands are thermally ablated without damaging the surrounding skin, the follicle root, or any other tissue surrounding the hair follicle.

18. The method of claim 1, wherein step b is repeated 2, 3, 4, 5, or 6 times prior to step c.

19. The method of claim 18, wherein the formulation comprising nanoshell particles is recycled from a prior performed step b and step b is repeated with the recycled formulation.

20. The method of claim 1, wherein in step c, the site is irradiated for a sufficient time period to thermally activate the nanoshell particles, and to modify or destroy the infundibula and sebaceous gland.

21. The method of claim 1, wherein the nanoshell particles comprise a silica core and a metal shell.

22. The method of claim 21, wherein the nanoshell particles further comprise an outer polyethylene glycol layer.

23. The method of claim 21, wherein the silica core is about 50 nm to about 500 nm thick.

24. The method of claim 21, wherein the metal is selected from the group consisting of gold, silver, copper, platinum, palladium, lead, iron, or combinations thereof.

25. The method of claim 21, wherein the metal shell is about 1 nm to about 100 nm thick.

26. The method of claim 1, wherein the nanoshell particles are thermally activated by a pulsed or continuous laser.

27. The method of claim 1, wherein the nanoshell particles absorb wavelengths ranging from about 700 nm to about 1100 nm.

28. The method of claim 20, wherein following step c, the opening to the infundibulum is modified.

29. The method of claim 20, wherein following step c, the sebaceous gland is modified.

30. The method of claim 20, wherein following step c, the sebaceous gland is destroyed.

31. The method of claim 1, wherein the sebaceous gland disorder is acne vulgaris, acne rosacea, or sebaceous gland hyperplasia.

32. The method of claim 31, wherein following step c, acne vulgaris is cured.
